SEQUENCE LISTING (654 sequences)

SEQ ID NO: 1 - protein, 662 aa, synthetic construct
  source 1..662: note = IL31RAv4
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI

SEQ ID NO: 2 - protein, 732 aa, synthetic construct
  source 1..732: note = IL31RAv3
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 3 - protein, 716 aa, Mus sp.
  source 1..716: note = Mouse-derived IL-31RA
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQEAILRIPDVHEKTFQYIKSMKAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSISVFEIVLLTSLVGGGLLLLSIKTVTFGLRKPNRLTPLCCPDVPNPAESSLATWLGDGFKKSNMKETGNSGDTEDVVLKPCPVPADLIDKLVVNFENFLEVVLTEEAGKGQASILGGEANEYVTSPSRPDGPPGKSFKEPSVLTEVASEDSHSTCSRMADEAYSELARQPSSSCQSPGLSPPREDQAQNPYLKNSVTTREFLVHENIPEHSKGEV

SEQ ID NO: 4 - protein, 735 aa, Macaca fascicularis
  source 1..735: note = Cynomolgus monkey-derived IL-31RA
MMWTWALWMFPLLCKFGLAALPAKPENISCVYYYRKNLTCTWSPGKETSYTQYTAKRTYAFGKKHDNCTTSSSTSENRASCSFFLPRITIPDNYTIEVEAENGDGVIKSDMTCWRLEDIAKTEPPEIFSVKPVLGIKRMIRIEWIKPELAPVSSDLKYALRFRTVNSTSWMEVNFAKNRKDTNQTYNLMGLQAFTEYVVALRCAVKESKFWSDWSQEKMGMTEEEAPCGLELWRVLKPTEVDGRRPVRLLWKKARGAPVLEKTLGYNIWYFPENNTNLTETVNTTNQQLELHLGGESYWVSMISYNSLGKSPVTTLRIPAIQEKSFRCIEVMQACLAEDQLVVKWQSSALDVNTWMIEWFPDMDSEHPTLSWESVSQATNWTIQQDKLKPFWCYNISVYPMLHDKVGEPYSIQAYAKEGIPSKGPETKVENIGVKTVTITWKEIPKSERKGIICNYTIFYQAEGGKGFSKTVNSSILQYGLESLKRKTSYTVRVMASTSAGGINGTSINFKTLSFSVFEIILITSLIGGGLLILIILTVAYGLKKPNKLTHLCWPSVPNPAESSIATWRGDDFKDKLNLKESDDSVNTEDRILKPCSTPSDKLVIDKSVVNFGNVLQEMFTDEARTGQENNLGGEKNEYVTHPFRADCPLGKSFEELPVSPEIPPRKSQYLRSRMPEGTCLEAEEQLLVSGQSLESLAPDHVREAAAPNPYLKNSVTTREFLVSQKLPEHTKGEV

SEQ ID NO: 5 - protein, 445 aa, synthetic construct
  source 1..445: note = Nemolizumab
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 6 - protein, 214 aa, synthetic construct
  source 1..214: note = Nemolizumab
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 121 aa, synthetic construct
  source 1..121: note = heavy chain variable region of nemolizumab
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSS

SEQ ID NO: 8 - protein, 5 aa, synthetic construct
  source 1..5: note = HCDR1 of nemolizumab
GYIMN

SEQ ID NO: 9 - protein, 17 aa, synthetic construct
  source 1..17: note = HCDR2
LINPYNGGTDYNPQFQD

SEQ ID NO: 10 - protein, 12 aa, synthetic construct
  source 1..12: note = HCDR3
DGYDDGPYTLET

SEQ ID NO: 11 - protein, 108 aa, synthetic construct
  source 1..108: note = light chain variable region of nemolizumab
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIKR

SEQ ID NO: 12 - protein, 11 aa, synthetic construct
  source 1..11: note = LCDR1 of nemolizumab
QASEDIYSFVA

SEQ ID NO: 13 - protein, 7 aa, synthetic construct
  source 1..7: note = LCDR2
NAQTEAQ

SEQ ID NO: 14 - protein, 9 aa, synthetic construct
  source 1..9: note = LCDR3
QHHYDSPLT

SEQ ID NO: 15 - protein, 11 aa, Homo sapiens
  source 1..11: note = EGR4_HUMAN.H10MO.D
GGSGGYRGGGM

SEQ ID NO: 16 - protein, 13 aa, Homo sapiens
  source 1..13: note = FLI1_HUMAN.H10MO.A
RCMGGAAGYGGSK

SEQ ID NO: 17 - protein, 22 aa, Homo sapiens
  source 1..22: note = ETV1_HUMAN.H10MO.B
SSVGRCMGGAAGYRVVSSBSSS

SEQ ID NO: 18 - protein, 14 aa, Homo sapiens
  source 1..14: note = ELF3_HUMAN.H10MO.D
GGSAAACAGGAARY

SEQ ID NO: 19 - protein, 22 aa, Homo sapiens
  source 1..22: note = SP2_HUMAN.H10MO.C
GGSSVGGGGGCGGGGCCDGSGS

SEQ ID NO: 20 - protein, 19 aa, Homo sapiens
  source 1..19: note = KLF16_HUMAN.H10MO.D
GGGGGYGKGKBGGGGGGGG

SEQ ID NO: 21 - protein, 19 aa, Homo sapiens
  source 1..19: note = CTCFL_HUMAN.H10MO.A
YBRCCASYAGRKGGCRSYV

SEQ ID NO: 22 - protein, 11 aa, Homo sapiens
  source 1..11: note = STAT3_HUMAN.H10MO.A
YTTCCYGGAAR

SEQ ID NO: 23 - protein, 11 aa, Homo sapiens
  source 1..11: note = ETS2_HUMAN.H10MO.C
VMVGGAAGTKS

SEQ ID NO: 24 - protein, 22 aa, Homo sapiens
  source 1..22: note = SP4_HUMAN.H10MO.D
GGGGGCGGGGCCKGGGSSGGGS

SEQ ID NO: 25 - protein, 12 aa, Homo sapiens
  source 1..12: note = ARNT2_HUMAN.H10MO.D
GNCGTGGSASRC

SEQ ID NO: 26 - protein, 10 aa, Homo sapiens
  source 1..10: note = GABP1_HUMAN.H10MO.C
CCGGAAGTGV

SEQ ID NO: 27 - protein, 10 aa, Homo sapiens
  source 1..10: note = GLIS3_HUMAN.H10MO.D
GYGGGGGGTM

SEQ ID NO: 28 - protein, 9 aa, Homo sapiens
  source 1..9: note = ZIC2_HUMAN.H10MO.C
KGGGTGGTC

SEQ ID NO: 29 - protein, 17 aa, Homo sapiens
  source 1..17: note = KLF15_HUMAN.H10MO.D
RSMKGGGAGDKGGGGSS

SEQ ID NO: 30 - protein, 9 aa, Homo sapiens
  source 1..9: note = ZIC1_HUMAN.H10MO.B
KGGGWGGTS

SEQ ID NO: 31 - protein, 20 aa, Homo sapiens
  source 1..20: note = TBX1_HUMAN.H10MO.D
GGGYGGGYAGGGGTGGGMDD

SEQ ID NO: 32 - protein, 17 aa, Homo sapiens
  source 1..17: note = ETV7_HUMAN.H10MO.D
KCMHCAGGAAGTRACDY

SEQ ID NO: 33 - protein, 22 aa, Homo sapiens
  source 1..22: note = TFDP1_HUMAN.H10MO.D
SSSVSSGSGSVRGGGGCGGGRV

SEQ ID NO: 34 - protein, 10 aa, Homo sapiens
  source 1..10: note = FOXJ2_HUMAN.H10MO.C
TGTTTRTTTW

SEQ ID NO: 35 - protein, 13 aa, Homo sapiens
  source 1..13: note = GABPA_HUMAN.H10MO.A
SVRCCGGAAGTGV

SEQ ID NO: 36 - protein, 11 aa, Homo sapiens
  source 1..11: note = FOSL2_HUMAN.H10MO.A
RTGACTCABHN

SEQ ID NO: 37 - protein, 19 aa, Homo sapiens
  source 1..19: note = TBX15_HUMAN.H10MO.D
GGGGGGGGGGGGGTGGGDR

SEQ ID NO: 38 - protein, 21 aa, Homo sapiens
  source 1..21: note = PTF1A_HUMAN.H10MO.C
CRGGRAAHWSMACASSTGYSC

SEQ ID NO: 39 - protein, 10 aa, Homo sapiens
  source 1..10: note = NR4A3_HUMAN.H10MO.D
CAAAGGTCAS

SEQ ID NO: 40 - protein, 11 aa, Homo sapiens
  source 1..11: note = KLF1_HUMAN.H10MO.C
CAGGGTGKGGC

SEQ ID NO: 41 - protein, 17 aa, Homo sapiens
  source 1..17: note = PURA_HUMAN.H10MO.D
GGWRKKGGDGGGCVKGG

SEQ ID NO: 42 - protein, 22 aa, Homo sapiens
  source 1..22: note = ZN143_HUMAN.H10MO.A
GCRWKGCATKMTGGGARDTVBH

SEQ ID NO: 43 - protein, 14 aa, Homo sapiens
  source 1..14: note = E2F1_HUMAN.H10MO.A
DDNDGGCGSGAAAV

SEQ ID NO: 44 - protein, 22 aa, Homo sapiens
  source 1..22: note = ZKSC1_HUMAN.H10MO.C
CCYGGCACAYAGTAGGTSCTCA

SEQ ID NO: 45 - protein, 20 aa, Homo sapiens
  source 1..20: note = SP3_HUMAN.H10MO.B
SGVVGGGGGCGGGGCBRGSS

SEQ ID NO: 46 - protein, 10 aa, Homo sapiens
  source 1..10: note = AP2B_HUMAN.H10MO.B
GCCYGVGGGS

SEQ ID NO: 47 - protein, 22 aa, Homo sapiens
  source 1..22: note = REST_HUMAN.H10MO.A
TTCAGCACCAYGGACAGCKCCM

SEQ ID NO: 48 - protein, 19 aa, Homo sapiens
  source 1..19: note = ZFX_HUMAN.H10MO.C
SVGSSSSSCAGGCCBVGSC

SEQ ID NO: 49 - protein, 11 aa, Homo sapiens
  source 1..11: note = RHXF1_HUMAN.H10MO.D
NRGGGATYADS

SEQ ID NO: 50 - protein, 12 aa, Homo sapiens
  source 1..12: note = KLF12_HUMAN.H10MO.D
DDGGGCGTGGYH

SEQ ID NO: 51 - protein, 14 aa, Homo sapiens
  source 1..14: note = NR1H4_HUMAN.H10MO.C
RRGGTCADTGACCT

SEQ ID NO: 52 - protein, 17 aa, Homo sapiens
  source 1..17: note = PROX1_HUMAN.H10MO.D
KGGKARGGCGKSKKGGG

SEQ ID NO: 53 - protein, 9 aa, Homo sapiens
  source 1..9: note = BHE40_HUMAN.H10MO.A
KCACGTGAS

SEQ ID NO: 54 - protein, 13 aa, Homo sapiens
  source 1..13: note = ERG_HUMAN.H10MO.B
RRACCGGAARTSV

SEQ ID NO: 55 - protein, 14 aa, Homo sapiens
  source 1..14: note = ERR1_HUMAN.H10MO.D
GSCCAAGGTCASVS

SEQ ID NO: 56 - protein, 14 aa, Homo sapiens
  source 1..14: note = AP2C_HUMAN.H10MO.A
VTGSCCTGRGGSHR

SEQ ID NO: 57 - protein, 19 aa, Homo sapiens
  source 1..19: note = SP1_HUMAN.H10MO.C
GGGRRGGGGGCGGGGCSDG

SEQ ID NO: 58 - protein, 20 aa, Homo sapiens
  source 1..20: note = P53_HUMAN.H10MO.B
RGRCATGTCTGGRCATGYCH

SEQ ID NO: 59 - protein, 13 aa, Homo sapiens
  source 1..13: note = ETV5_HUMAN.H10MO.D
GHCAGGAAGWAAY

SEQ ID NO: 60 - protein, 15 aa, Homo sapiens
  source 1..15: note = COE1_HUMAN.H10MO.A
DRKYCCCWGGGGAVH

SEQ ID NO: 61 - protein, 22 aa, Homo sapiens
  source 1..22: note = MAZ_HUMAN.H10MO.A
GGGMGGGGSSGGGGGGGGGGGG

SEQ ID NO: 62 - protein, 17 aa, Homo sapiens
  source 1..17: note = NR2E1_HUMAN.H10MO.D
DMTTGACTTWTTGACTT

SEQ ID NO: 63 - protein, 10 aa, Homo sapiens
  source 1..10: note = HXB1_HUMAN.H10MO.D
TGATKGATGG

SEQ ID NO: 64 - protein, 12 aa, Homo sapiens
  source 1..12: note = FOXK1_HUMAN.H10MO.D
KRTTGKGTSSGT

SEQ ID NO: 65 - protein, 24 aa, Homo sapiens
  source 1..24: note = ZN350_HUMAN.H10MO.C
ASGGGVVGCARDNVTTTGKKGCCC

SEQ ID NO: 66 - protein, 13 aa, Homo sapiens
  source 1..13: note = NR6A1_HUMAN.H10MO.B
AAGKTCAAGKTCA

SEQ ID NO: 67 - protein, 18 aa, Homo sapiens
  source 1..18: note = ESR2_HUMAN.H10MO.A
VRGGTCASKSTGACCTKR

SEQ ID NO: 68 - protein, 14 aa, Homo sapiens
  source 1..14: note = ZEP2_HUMAN.H10MO.D
GGYDGGGWAACCSS

SEQ ID NO: 69 - protein, 11 aa, Homo sapiens
  source 1..11: note = GLI1_HUMAN.H10MO.C
BTGGGTGGTCY

SEQ ID NO: 70 - protein, 10 aa, Homo sapiens
  source 1..10: note = KLF4_HUMAN.H10MO.A
DGGGYGKGGC

SEQ ID NO: 71 - protein, 14 aa, Homo sapiens
  source 1..14: note = E2F4_HUMAN.H10MO.A
NDDKGGCGSGAAAN

SEQ ID NO: 72 - protein, 11 aa, Homo sapiens
  source 1..11: note = STA5B_HUMAN.H10MO.C
YTTCTKRGAAR

SEQ ID NO: 73 - protein, 16 aa, Homo sapiens
  source 1..16: note = EVI1_HUMAN.H10MO.B
WAGAYAAGATAANAKA

SEQ ID NO: 74 - protein, 9 aa, Homo sapiens
  source 1..9: note = JUN_HUMAN.H10MO.A
VTGAGTCAB

SEQ ID NO: 75 - protein, 20 aa, Homo sapiens
  source 1..20: note = RGRCATGYYTGGRCWTGYYB
RGRCATGYYTGGRCWTGYYB

SEQ ID NO: 76 - protein, 15 aa, Homo sapiens
  source 1..15: note = ZN148_HUMAN.H10MO.D
KGRGKGGGGGAGGGG

SEQ ID NO: 77 - protein, 11 aa, Homo sapiens
  source 1..11: note = ETV6_HUMAN.H10MO.D
WVCCGGAAGTR

SEQ ID NO: 78 - protein, 11 aa, Homo sapiens
  source 1..11: note = STAT4_HUMAN.H10MO.D
YTTCYYRGAAR

SEQ ID NO: 79 - protein, 7 aa, Homo sapiens
  source 1..7: note = GCR_HUMAN.H10MO.S
AGAACAK

SEQ ID NO: 80 - protein, 9 aa, Homo sapiens
  source 1..9: note = ETS1_HUMAN.H10MO.C
ACMGGAAGT

SEQ ID NO: 81 - protein, 13 aa, Homo sapiens
  source 1..13: note = NR2C2_HUMAN.H10MO.A
RGGTCARAGGTCA

SEQ ID NO: 82 - protein, 16 aa, Homo sapiens
  source 1..16: note = TGIF1_HUMAN.H10MO.D
TGTSAGGTGTCASCTK

SEQ ID NO: 83 - protein, 14 aa, Homo sapiens
  source 1..14: note = TEAD1_HUMAN.H10MO.D
YACATTCCWSMKSH

SEQ ID NO: 84 - protein, 18 aa, Homo sapiens
  source 1..18: note = ESR1_HUMAN.H10MO.S
RGGTCASVSTGACCTGRS

SEQ ID NO: 85 - protein, 18 aa, Homo sapiens
  source 1..18: note = EGR1_HUMAN.H10MO.A
GGGRDGYGKGGGHGGGRG

SEQ ID NO: 86 - protein, 9 aa, Homo sapiens
  source 1..9: note = PPARG_HUMAN.H10MO.S
MAWAGGTCA

SEQ ID NO: 87 - skipped

SEQ ID NO: 88 - protein, 11 aa, Homo sapiens
  source 1..11: note = TFE2_HUMAN.H10MO.C
AMCAGATGBYC

SEQ ID NO: 89 - protein, 14 aa, Homo sapiens
  source 1..14: note = HSF1_HUMAN.H10MO.A
RGAANVTTCYAGAA

SEQ ID NO: 90 - protein, 12 aa, Homo sapiens
  source 1..12: note = GRHL1_HUMAN.H10MO.D
AACCGGTTTDDC

SEQ ID NO: 91 - protein, 22 aa, Homo sapiens
  source 1..22: note = RREB1_HUMAN.H10MO.D
DKGKKKGKGGKTGKTTTGGGKT

SEQ ID NO: 92 - protein, 12 aa, Homo sapiens
  source 1..12: note = IRF9_HUMAN.H10MO.C
GAAAGCGAAAYT

SEQ ID NO: 93 - protein, 21 aa, Homo sapiens
  source 1..21: note = MAFA_HUMAN.H10MO.D
STGCTGACBMYGCAGYHTYCV

SEQ ID NO: 94 - protein, 15 aa, Homo sapiens
  source 1..15: note = AP2A_HUMAN.H10MO.C
ATKSCCTGRGGSHAD

SEQ ID NO: 95 - protein, 19 aa, Homo sapiens
  source 1..19: note = NR1H2_HUMAN.H10MO.D
TAAAGGTCAAAGGTCAACK

SEQ ID NO: 96 - protein, 10 aa, Homo sapiens
  source 1..10: note = THB_HUMAN.H10MO.S
SGTSAGGTCA

SEQ ID NO: 97 - protein, 21 aa, Homo sapiens
  source 1..21: note = MAFG_HUMAN.H10MO.C
AAAANTGCTGASTCAGCADWW

SEQ ID NO: 98 - protein, 17 aa, Homo sapiens
  source 1..17: note = PLAG1_HUMAN.H10MO.D
GGRGGSMHNWVKAGGGG

SEQ ID NO: 99 - protein, 8 aa, Homo sapiens
  source 1..8: note = SOX13_HUMAN.H10MO.D
YATTGTTY

SEQ ID NO: 100 - protein, 14 aa, Homo sapiens
  source 1..14: note = GLIS2_HUMAN.H10MO.D
CKTCGCGGGGGGTM

SEQ ID NO: 101 - protein, 10 aa, Homo sapiens
  source 1..10: note = PLAL1_HUMAN.H10MO.D
CRGGGGGCCC

SEQ ID NO: 102 - protein, 17 aa, Homo sapiens
  source 1..17: note = THB_HUMAN.H10MO.C
GRSSYSASVTSAGGTCA

SEQ ID NO: 103 - protein, 13 aa, Homo sapiens
  source 1..13: note = WT1_HUMAN.H10MO.D
GMGGGGGCGKGGG

SEQ ID NO: 104 - protein, 12 aa, Homo sapiens
  source 1..12: note = NF2L2_HUMAN.H10MO.D
VRTGACTCAGCA

SEQ ID NO: 105 - protein, 11 aa, Homo sapiens
  source 1..11: note = GLI2_HUMAN.H10MO.B
GTGGGTGGTCY

SEQ ID NO: 106 - protein, 12 aa, Homo sapiens
  source 1..12: note = KAISO_HUMAN.H10MO.S
YTSGCRGGARKH

SEQ ID NO: 107 - protein, 20 aa, Homo sapiens
  source 1..20: note = CTCF_HUMAN.H10MO.A
YKRCCASYAGRKGGCRSYVB

SEQ ID NO: 108 - protein, 15 aa, Homo sapiens
  source 1..15: note = STAT1_HUMAN.H10MO.S
SDYTTCCRGGAAAYS

SEQ ID NO: 109 - protein, 11 aa, Homo sapiens
  source 1..11: note = FOXF1_HUMAN.H10MO.D
WTGTTTATTTW

SEQ ID NO: 110 - protein, 22 aa, Homo sapiens
  source 1..22: note = THAP1_HUMAN.H10MO.D
SGCCGCCATSTYGGSYBCGGGC

SEQ ID NO: 111 - protein, 13 aa, Homo sapiens
  source 1..13: note = STAT6_HUMAN.H10MO.C
TTCYYHGGAAWYY

SEQ ID NO: 112 - protein, 11 aa, Homo sapiens
  source 1..11: note = STA5A_HUMAN.H10MO.B
YTTCYKRGAAW

SEQ ID NO: 113 - protein, 13 aa, Homo sapiens
  source 1..13: note = PLAG1_HUMAN.H10MO.S
ARDGGGGGSHBYM

SEQ ID NO: 114 - protein, 10 aa, Homo sapiens
  source 1..10: note = USF1_HUMAN.H10MO.A
GGTCACGTGR

SEQ ID NO: 115 - protein, 11 aa, Homo sapiens
  source 1..11: note = JUND_HUMAN.H10MO.A
RRTGAGTCAYH

SEQ ID NO: 116 - protein, 20 aa, Homo sapiens
  source 1..20: note = ERF_HUMAN.H10MO.D
WASAKKWSKTWSGRBAYTTC

SEQ ID NO: 117 - protein, 12 aa, Homo sapiens
  source 1..12: note = INSM1_HUMAN.H10MO.C
TGTMAGGGGGCR

SEQ ID NO: 118 - protein, 12 aa, Homo sapiens
  source 1..12: note = ZN219_HUMAN.H10MO.D
GDGGGGGGYGGA

SEQ ID NO: 119 - protein, 8 aa, Homo sapiens
  source 1..8: note = SNAI1_HUMAN.H10MO.C
SCAGGTGK

SEQ ID NO: 120 - protein, 20 aa, Homo sapiens
  source 1..20: note = CLOCK_HUMAN.H10MO.D
GGDBKGGDGRSRCGKGTRGK

SEQ ID NO: 121 - protein, 21 aa, Homo sapiens
  source 1..21: note = PAX5_HUMAN.H10MO.A
SSVDRVKCAGCSRAGCGTGAC

SEQ ID NO: 122 - protein, 11 aa, Homo sapiens
  source 1..11: note = GATA2_HUMAN.H10MO.A
RSAGATAAGVV

SEQ ID NO: 123 - protein, 10 aa, Homo sapiens
  source 1..10: note = HXA1_HUMAN.H10MO.C
TKGATGGATG

SEQ ID NO: 124 - protein, 14 aa, Homo sapiens
  source 1..14: note = SPDEF_HUMAN.H10MO.D
RWCCMGGAWGWAGT

SEQ ID NO: 125 - protein, 7 aa, Homo sapiens
  source 1..7: note = MYF6_HUMAN.H10MO.C
GCAGSTG

SEQ ID NO: 126 - protein, 10 aa, Homo sapiens
  source 1..10: note = MYBB_HUMAN.H10MO.D
TRSMGGTTRW

SEQ ID NO: 127 - protein, 11 aa, Homo sapiens
  source 1..11: note = YBOX1_HUMAN.H10MO.D
BSKGATTSSCY

SEQ ID NO: 128 - protein, 13 aa, Homo sapiens
  source 1..13: note = NR2C1_HUMAN.H10MO.C
GSYVAGAGGTCAB

SEQ ID NO: 129 - protein, 8 aa, Homo sapiens
  source 1..8: note = ETV4_HUMAN.H10MO.B
SAGGAAGT

SEQ ID NO: 130 - protein, 10 aa, Homo sapiens
  source 1..10: note = NKX21_HUMAN.H10MO.D
STCAAGKGCH

SEQ ID NO: 131 - DNA, 11 bp, Homo sapiens
  source 1..11: mol_type = other DNA; note = SP1_HUMAN.H10MO.S
gggggcggggc

SEQ ID NO: 132 - protein, 11 aa, Homo sapiens
  source 1..11: note = ZEB1_HUMAN.H10MO.B
NVCAGGTRDRN

SEQ ID NO: 133 - protein, 7 aa, Homo sapiens
  source 1..7: note = PAX2_HUMAN.H10MO.S
SMRTGAC

SEQ ID NO: 134 - protein, 9 aa, Homo sapiens
  source 1..9: note = ZIC3_HUMAN.H10MO.C
BGGGTGGYC

SEQ ID NO: 135 - protein, 8 aa, Homo sapiens
  source 1..8: note = IKZF1_HUMAN.H10MO.C
BTGGGARR

SEQ ID NO: 136 - protein, 12 aa, Homo sapiens
  source 1..12: note = P73_HUMAN.H10MO.S
GGGRCADGYYYB

SEQ ID NO: 137 - protein, 14 aa, Homo sapiens
  source 1..14: note = HXB3_HUMAN.H10MO.D
YTAATTASYTTTTW

SEQ ID NO: 138 - protein, 12 aa, Homo sapiens
  source 1..12: note = E2F6_HUMAN.H10MO.C
NKGGCGGGARRG

SEQ ID NO: 139 - protein, 18 aa, Homo sapiens
  source 1..18: note = NR1I3_HUMAN.H10MO.C
RRGGTCARVRRAGTTCAD

SEQ ID NO: 140 - protein, 19 aa, Homo sapiens
  source 1..19: note = SOX18_HUMAN.H10MO.D
SHWHVMWTTGTTBTYKTCC

SEQ ID NO: 141 - protein, 9 aa, Homo sapiens
  source 1..9: note = ATF3_HUMAN.H10MO.A
RTGACKYMA

SEQ ID NO: 142 - protein, 11 aa, Homo sapiens
  source 1..11: note = NRL_HUMAN.H10MO.D
TGCTGACKYWR

SEQ ID NO: 143 - protein, 9 aa, Homo sapiens
  source 1..9: note = HBP1_HUMAN.H10MO.D
AYYCATTGA

SEQ ID NO: 144 - protein, 15 aa, Homo sapiens
  source 1..15: note = FOXC1_HUMAN.H10MO.C
CDWTGTTTACTTWRG

SEQ ID NO: 145 - protein, 12 aa, Homo sapiens
  source 1..12: note = EVX2_HUMAN.H10MO.A
TTTTATKRCHYH

SEQ ID NO: 146 - protein, 14 aa, Homo sapiens
  source 1..14: note = PAX8_HUMAN.H10MO.D
BTVAYTSRMGYRKR

SEQ ID NO: 147 - protein, 11 aa, Homo sapiens
  source 1..11: note = GLI3_HUMAN.H10MO.B
BTGGGTGGTCY

SEQ ID NO: 148 - protein, 12 aa, Homo sapiens
  source 1..12: note = ATF6A_HUMAN.H10MO.B
GKGSTGACGTGG

SEQ ID NO: 149 - protein, 14 aa, Homo sapiens
  source 1..14: note = PO2F3_HUMAN.H10MO.D
DTYATGCWWAWTTR

SEQ ID NO: 150 - protein, 12 aa, Homo sapiens
  source 1..12: note = COT1_HUMAN.H10MO.S
GKBCAAAGGTCA

SEQ ID NO: 151 - protein, 8 aa, Homo sapiens
  source 1..8: note = PAX5_HUMAN.H10MO.S
GGCTGAGS

SEQ ID NO: 152 - DNA, 12 bp, Homo sapiens
  source 1..12: mol_type = other DNA; note = ZSCA4_HUMAN.H10MO.D
agtgtgtgcact

SEQ ID NO: 153 - protein, 14 aa, Homo sapiens
  source 1..14: note = DLX5_HUMAN.H10MO.D
AATTWDNBDTAATT

SEQ ID NO: 154 - protein, 12 aa, Homo sapiens
  source 1..12: note = NDF1_HUMAN.H10MO.C
CGGCAGRTGKSC

SEQ ID NO: 155 - protein, 7 aa, Homo sapiens
  source 1..7: note = SNAI2_HUMAN.H10MO.C
BCAGGTG

SEQ ID NO: 156 - protein, 11 aa, Homo sapiens
  source 1..11: note = BCL6B_HUMAN.H10MO.D
HYGCTTTCKAG

SEQ ID NO: 157 - DNA, 10 bp, Homo sapiens
  source 1..10: mol_type = other DNA; note = HXD10_HUMAN.H10MO.D
tgctttaatt

SEQ ID NO: 158 - protein, 10 aa, Homo sapiens
  source 1..10: note = TWST1_HUMAN.H10MO.D
MCCCAGGTGK

SEQ ID NO: 159 - protein, 9 aa, Homo sapiens
  source 1..9: note = ELK1_HUMAN.H10MO.A
RCCGGAAGT

SEQ ID NO: 160 - protein, 12 aa, Homo sapiens
  source 1..12: note = REL_HUMAN.H10MO.C
DKGGRNWTTCCM

SEQ ID NO: 161 - protein, 17 aa, Homo sapiens
  source 1..17: note = GATA5_HUMAN.H10MO.D
WVANWGATAAYTYRRHK

SEQ ID NO: 162 - protein, 12 aa, Homo sapiens
  source 1..12: note = PAX4_HUMAN.H10MO.D
TWAYTAATTMRH

SEQ ID NO: 163 - protein, 7 aa, Homo sapiens
  source 1..7: note = GATA6_HUMAN.H10MO.S
NWGATAA

SEQ ID NO: 164 - protein, 16 aa, Homo sapiens
  source 1..16: note = SOX2_HUMAN.H10MO.B
CCWTTGTYATGCAAAW

SEQ ID NO: 165 - protein, 13 aa, Homo sapiens
  source 1..13: note = TBR1_HUMAN.H10MO.D
RAAGGTGTGAAAW

SEQ ID NO: 166 - protein, 11 aa, Homo sapiens
  source 1..11: note = FOS_HUMAN.H10MO.A
RTGAGTCAYYN

SEQ ID NO: 167 - protein, 8 aa, Homo sapiens
  source 1..8: note = CDX1_HUMAN.H10MO.C
HTTTATGD

SEQ ID NO: 168 - protein, 11 aa, Homo sapiens
  source 1..11: note = NFKB2_HUMAN.H10MO.D
GRAATBYCCCT

SEQ ID NO: 169 - protein, 14 aa, Homo sapiens
  source 1..14: note = PBX3_HUMAN.H10MO.B
YBYTGATTGRYTGR

SEQ ID NO: 170 - protein, 11 aa, Homo sapiens
  source 1..11: note = ELF5_HUMAN.H10MO.D
AHMAGGAWRTW

SEQ ID NO: 171 - protein, 10 aa, Homo sapiens
  source 1..10: note = BATF_HUMAN.H10MO.S
MTGAGTCAKW

SEQ ID NO: 172 - protein, 19 aa, Homo sapiens
  source 1..19: note = TAL1_HUMAN.H10MO.A
CTGKNNNNNDSWGATAAGV

SEQ ID NO: 173 - protein, 9 aa, Homo sapiens
  source 1..9: note = KLF8_HUMAN.H10MO.C
CAGGGKGTG

SEQ ID NO: 174 - protein, 11 aa, Homo sapiens
  source 1..11: note = MESP1_HUMAN.H10MO.D
VCAGGTGKKBD

SEQ ID NO: 175 - protein, 12 aa, Homo sapiens
  source 1..12: note = TBX21_HUMAN.H10MO.D
RAGGTGTGAAWT

SEQ ID NO: 176 - protein, 17 aa, Homo sapiens
  source 1..17: note = FOXB1_HUMAN.H10MO.D
RTMAATRTTTACATAAB

SEQ ID NO: 177 - protein, 15 aa, Homo sapiens
  source 1..15: note = PBX1_HUMAN.H10MO.B
WAWTTGATTGATGDB

SEQ ID NO: 178 - protein, 20 aa, Homo sapiens
  source 1..20: note = RFX5_HUMAN.H10MO.A
STCMBCWGTTGCYRGGVARA

SEQ ID NO: 179 - protein, 17 aa, Homo sapiens
  source 1..17: note = HEY2_HUMAN.H10MO.D
GKBGGCWCGTGGCHTBV

SEQ ID NO: 180 - protein, 13 aa, Homo sapiens
  source 1..13: note = SPIC_HUMAN.H10MO.D
KVDRRWGGGKDRG

SEQ ID NO: 181 - protein, 19 aa, Homo sapiens
  source 1..19: note = USF2_HUMAN.H10MO.A
GGGTCACGTGRBSVSSSSB

SEQ ID NO: 182 - protein, 17 aa, Homo sapiens
  source 1..17: note = ESR1_HUMAN.H10MO.A
MMAGGTCASVVTGHCCH

SEQ ID NO: 183 - protein, 16 aa, Homo sapiens
  source 1..16: note = SPZ1_HUMAN.H10MO.D
CGGCKGWWACMBYGGG

SEQ ID NO: 184 - protein, 9 aa, Homo sapiens
  source 1..9: note = E4F1_HUMAN.H10MO.D
YGTKACGTC

SEQ ID NO: 185 - protein, 14 aa, Homo sapiens
  source 1..14: note = IRF2_HUMAN.H10MO.C
SGAAAGYGAAASYR

SEQ ID NO: 186 - protein, 11 aa, Homo sapiens
  source 1..11: note = ATF1_HUMAN.H10MO.B
NRTGACGTMAK

SEQ ID NO: 187 - protein, 14 aa, Homo sapiens
  source 1..14: note = NFIL3_HUMAN.H10MO.C
RTTAYGTAAYKYMB

SEQ ID NO: 188 - skipped

SEQ ID NO: 189 - protein, 13 aa, Homo sapiens
  source 1..13: note = GLIS1_HUMAN.H10MO.D
TCGTGGGGGGTMD

SEQ ID NO: 190 - DNA, 12 bp, Homo sapiens
  source 1..12: mol_type = other DNA; note = HES7_HUMAN.H10MO.D
tggcacgtgccg

SEQ ID NO: 191 - protein, 11 aa, Homo sapiens
  source 1..11: note = GSC2_HUMAN.H10MO.D
RKMGGRTTRGB

SEQ ID NO: 192 - protein, 11 aa, Homo sapiens
  source 1..11: note = CREM_HUMAN.H10MO.C
CRVTGACGTCA

SEQ ID NO: 193 - protein, 17 aa, Homo sapiens
  source 1..17: note = RXRA_HUMAN.H10MO.C
MWSTRGGDCAAAGGTCA

SEQ ID NO: 194 - protein, 17 aa, Homo sapiens
  source 1..17: note = BARX1_HUMAN.H10MO.D
TAATYGNTWTTTAATKG

SEQ ID NO: 195 - protein, 12 aa, Homo sapiens
  source 1..12: note = SMAD1_HUMAN.H10MO.D
RSCCTGTCTGCC

SEQ ID NO: 196 - protein, 11 aa, Homo sapiens
  source 1..11: note = TGIF2_HUMAN.H10MO.D
DTGACAGCTGT

SEQ ID NO: 197 - protein, 19 aa, Homo sapiens
  source 1..19: note = MLXPL_HUMAN.H10MO.D
GCAYGKGVCAMCSBCGTGB

SEQ ID NO: 198 - protein, 13 aa, Homo sapiens
  source 1..13: note = NFYA_HUMAN.H10MO.A
YRRCCAATSAGMR

SEQ ID NO: 199 - protein, 8 aa, Homo sapiens
  source 1..8: note = KLF6_HUMAN.H10MO.D
GGGGGCKG

SEQ ID NO: 200 - protein, 15 aa, Homo sapiens
  source 1..15: note = NFAT5_HUMAN.H10MO.D
SYGGAAAANTYCMTK

SEQ ID NO: 201 - protein, 8 aa, Homo sapiens
  source 1..8: note = NANOG_HUMAN.H10MO.S
KTTAATGG

SEQ ID NO: 202 - DNA, 11 bp, Homo sapiens
  source 1..11: mol_type = other DNA; note = CR3L1_HUMAN.H10MO.D
gccacgtggca

SEQ ID NO: 203 - protein, 13 aa, Homo sapiens
  source 1..13: note = PO6F1_HUMAN.H10MO.D
CATAAWTTATGCR

SEQ ID NO: 204 - protein, 11 aa, Homo sapiens
  source 1..11: note = EGR2_HUMAN.H10MO.C
DGMGTGGGCGG

SEQ ID NO: 205 - protein, 15 aa, Homo sapiens
  source 1..15: note = ZIC4_HUMAN.H10MO.D
DCRCAGCGGGGGGTC

SEQ ID NO: 206 - protein, 11 aa, Homo sapiens
  source 1..11: note = NFKB1_HUMAN.H10MO.B
GGGRAAKYCCC

SEQ ID NO: 207 - protein, 19 aa, Homo sapiens
  source 1..19: note = NR1I2_HUMAN.H10MO.C
DDRGGTYAANNRAGTTCAB

SEQ ID NO: 208 - protein, 11 aa, Homo sapiens
  source 1..11: note = OLIG2_HUMAN.H10MO.D
RCCATATGKTT

SEQ ID NO: 209 - protein, 8 aa, Homo sapiens
  source 1..8: note = SOX5_HUMAN.H10MO.C
YATTGTTW

SEQ ID NO: 210 - protein, 15 aa, Homo sapiens
  source 1..15: note = NFIA_HUMAN.H10MO.C
TTGGCHNRNWGCCAR

SEQ ID NO: 211 - protein, 11 aa, Homo sapiens
  source 1..11: note = HXC13_HUMAN.H10MO.D
DDTTTTACKAG

SEQ ID NO: 212 - protein, 7 aa, Homo sapiens
  source 1..7: note = ESR2_HUMAN.H10MO.S
RGGTCAS

SEQ ID NO: 213 - protein, 16 aa, Homo sapiens
  source 1..16: note = BCL6_HUMAN.H10MO.C
DARDRCTTTCYWGGAA

SEQ ID NO: 214 - protein, 18 aa, Homo sapiens
  source 1..18: note = ZN524_HUMAN.H10MO.D
GGGTDCRAGGGTTCGAGK

SEQ ID NO: 215 - protein, 17 aa, Homo sapiens
  source 1..17: note = ELF2_HUMAN.H10MO.C
VRRVSCGGAAGYRSVRS

SEQ ID NO: 216 - protein, 9 aa, Homo sapiens
  source 1..9: note = MYOD1_HUMAN.H10MO.C
SCASCTGTY

SEQ ID NO: 217 - protein, 14 aa, Homo sapiens
  source 1..14: note = HXA9_HUMAN.H10MO.D
TGACAGTTTWAYGR

SEQ ID NO: 218 - protein, 20 aa, Homo sapiens
  source 1..20: note = EVX1_HUMAN.H10MO.D
TDHTWDTWTGTTTACTTAWM

SEQ ID NO: 219 - protein, 12 aa, Homo sapiens
  source 1..12: note = FUBP1_HUMAN.H10MO.D
TYGTNTTTTTTT

SEQ ID NO: 220 - protein, 7 aa, Homo sapiens
  source 1..7: note = TGIF1_HUMAN.H10MO.S
NTGACAG

SEQ ID NO: 221 - protein, 13 aa, Homo sapiens
  source 1..13: note = SMAD4_HUMAN.H10MO.C
STGTCTSDCACCT

SEQ ID NO: 222 - skipped

SEQ ID NO: 223 - protein, 11 aa, Homo sapiens
  source 1..11: note = TLX1_HUMAN.H10MO.S
YGBYAAKRWGS

SEQ ID NO: 224 - protein, 16 aa, Homo sapiens
  source 1..16: note = TFCP2_HUMAN.H10MO.D
SCCTGMNCDSRCCRGA

SEQ ID NO: 225 - protein, 17 aa, Homo sapiens
  source 1..17: note = HMX1_HUMAN.H10MO.D
DTTAATTGHTTTTTAAT

SEQ ID NO: 226 - protein, 7 aa, Homo sapiens
  source 1..7: note = PRGR_HUMAN.H10MO.S
AGAACAB

SEQ ID NO: 227 - protein, 13 aa, Homo sapiens
  source 1..13: note = SCRT1_HUMAN.H10MO.D
KCAACAGGTGGTH

SEQ ID NO: 228 - protein, 11 aa, Homo sapiens
  source 1..11: note = FOSL1_HUMAN.H10MO.A
DRTGACTCATN

SEQ ID NO: 229 - protein, 11 aa, Homo sapiens
  source 1..11: note = DUXA_HUMAN.H10MO.D
TGATTARRTYA

SEQ ID NO: 230 - protein, 12 aa, Homo sapiens
  source 1..12: note = RORA_HUMAN.H10MO.B
WWAWBTAGGTCA

SEQ ID NO: 231 - protein, 11 aa, Homo sapiens
  source 1..11: note = HXB8_HUMAN.H10MO.C
TTGATTAATKV

SEQ ID NO: 232 - protein, 7 aa, Homo sapiens
  source 1..7: note = PRRX1_HUMAN.H10MO.D
TAAYCTG

SEQ ID NO: 233 - protein, 19 aa, Homo sapiens
  source 1..19: note = IRX2_HUMAN.H10MO.D
RCDKGKBDKGDCRKGTDVK

SEQ ID NO: 234 - protein, 8 aa, Homo sapiens
  source 1..8: note = OTX1_HUMAN.H10MO.D
AGGATTAV

SEQ ID NO: 235 - protein, 11 aa, Homo sapiens
  source 1..11: note = ATF7_HUMAN.H10MO.D
KRTGACGTCAT

SEQ ID NO: 236 - protein, 10 aa, Homo sapiens
  source 1..10: note = HXB7_HUMAN.H10MO.C
TTGATTRATK

SEQ ID NO: 237 - protein, 11 aa, Homo sapiens
  source 1..11: note = FOXH1_HUMAN.H10MO.A
TGTGGATTSNR

SEQ ID NO: 238 - protein, 16 aa, Homo sapiens
  source 1..16: note = HESX1_HUMAN.H10MO.D
ABSYGGCWMRTRRMMT

SEQ ID NO: 239 - protein, 13 aa, Homo sapiens
  source 1..13: note = COT2_HUMAN.H10MO.A
CARAGGTCARRGD

SEQ ID NO: 240 - protein, 14 aa, Homo sapiens
  source 1..14: note = BMAL1_HUMAN.H10MO.C
KGRACACGTGACYC

SEQ ID NO: 241 - protein, 14 aa, Homo sapiens
  source 1..14: note = AP2D_HUMAN.H10MO.D
CGCCYGVGGCSCGT

SEQ ID NO: 242 - protein, 20 aa, Homo sapiens
  source 1..20: note = HEN1_HUMAN.H10MO.C
BGGGKMKCAGCTGCGKCCYY

SEQ ID NO: 243 - protein, 13 aa, Homo sapiens
  source 1..13: note = NFAC1_HUMAN.H10MO.S
HGGAAAWTTCCAT

SEQ ID NO: 244 - protein, 12 aa, Homo sapiens
  source 1..12: note = CEBPE_HUMAN.H10MO.A
TTKCGHAATMTB

SEQ ID NO: 245 - protein, 8 aa, Homo sapiens
  source 1..8: note = CRX_HUMAN.H10MO.C
VRGATTAR

SEQ ID NO: 246 - protein, 13 aa, Homo sapiens
  source 1..13: note = EHF_HUMAN.H10MO.S
MWACCMGGAARTA

SEQ ID NO: 247 - protein, 15 aa, Homo sapiens
  source 1..15: note = FOXD1_HUMAN.H10MO.D
CWWTGTTTACHTWVR

SEQ ID NO: 248 - protein, 17 aa, Homo sapiens
  source 1..17: note = ZBT7A_HUMAN.H10MO.D
MKCWSKGGGTNBMCBAG

SEQ ID NO: 249 - protein, 16 aa, Homo sapiens
  source 1..16: note = PRDM4_HUMAN.H10MO.D
KBGGGGGGCCTTGAAA

SEQ ID NO: 250 - protein, 12 aa, Homo sapiens
  source 1..12: note = NFAC1_HUMAN.H10MO.A
MMATGGAAAWTT

SEQ ID NO: 251 - protein, 16 aa, Homo sapiens
  source 1..16: note = VDR_HUMAN.H10MO.B
RRGGTCASNGRGKTCA

SEQ ID NO: 252 - protein, 11 aa, Homo sapiens
  source 1..11: note = ID4_HUMAN.H10MO.D
DNCAGGTGYNV

SEQ ID NO: 253 - protein, 12 aa, Homo sapiens
  source 1..12: note = ALX1_HUMAN.H10MO.B
RTAATTRVATTA

SEQ ID NO: 254 - protein, 11 aa, Homo sapiens
  source 1..11: note = TF65_HUMAN.H10MO.A
GGGRMTTTCCM

SEQ ID NO: 255 - protein, 13 aa, Homo sapiens
  source 1..13: note = PAX3_HUMAN.H10MO.D
GWTAATYGATTAW

SEQ ID NO: 256 - protein, 11 aa, Homo sapiens
  source 1..11: note = FIGLA_HUMAN.H10MO.D
DHMCAGGTGKT

SEQ ID NO: 257 - protein, 9 aa, Homo sapiens
  source 1..9: note = NFAC3_HUMAN.H10MO.B
TGGAAAAHY

SEQ ID NO: 258 - protein, 15 aa, Homo sapiens
  source 1..15: note = FOXC2_HUMAN.H10MO.D
GTTTGTTTWSHYAGR

SEQ ID NO: 259 - protein, 11 aa, Homo sapiens
  source 1..11: note = FOSB_HUMAN.H10MO.C
GRTGACTCABH

SEQ ID NO: 260 - protein, 17 aa, Homo sapiens
  source 1..17: note = TLX1_HUMAN.H10MO.D
YYGGYAMBDDGCCAAGR

SEQ ID NO: 261 - protein, 14 aa, Homo sapiens
  source 1..14: note = ELF1_HUMAN.H10MO.A
GAACCMGGAAGTGS

SEQ ID NO: 262 - protein, 24 aa, Homo sapiens
  source 1..24: note = TBX3_HUMAN.H10MO.D
AGKWAGRSAMTTAGGTSWKAARAA

SEQ ID NO: 263 - protein, 11 aa, Homo sapiens
  source 1..11: note = HTF4_HUMAN.H10MO.B
VNSCAGGTGBG

SEQ ID NO: 264 - protein, 16 aa, Homo sapiens
  source 1..16: note = MEOX1_HUMAN.H10MO.D
GGTAATTAGBGKHAWW

SEQ ID NO: 265 - protein, 10 aa, Homo sapiens
  source 1..10: note = NR0B1_HUMAN.H10MO.D
GMGKGGGASR

SEQ ID NO: 266 - protein, 14 aa, Homo sapiens
  source 1..14: note = GATA6_HUMAN.H10MO.B
DDVAGATAAGAWAR

SEQ ID NO: 267 - protein, 20 aa, Homo sapiens
  source 1..20: note = RARG_HUMAN.H10MO.C
GGRGKTCRVCNRVAGGTCAV

SEQ ID NO: 268 - protein, 14 aa, Homo sapiens
  source 1..14: note = HXC8_HUMAN.H10MO.D
YTTSATTRATDSSC

SEQ ID NO: 269 - protein, 15 aa, Homo sapiens
  source 1..15: note = DLX6_HUMAN.H10MO.D
TAATTANNNHWWWWW

SEQ ID NO: 270 - protein, 11 aa, Homo sapiens
  source 1..11: note = HMX3_HUMAN.H10MO.D
NNTTAATTGST

SEQ ID NO: 271 - protein, 15 aa, Homo sapiens
  source 1..15: note = STAT2_HUMAN.H10MO.B
TYASTTTCNBTTYCH

SEQ ID NO: 272 - protein, 14 aa, Homo sapiens
  source 1..14: note = NFYC_HUMAN.H10MO.B
YAGCCAATSAGVGS

SEQ ID NO: 273 - protein, 11 aa, Homo sapiens
  source 1..11: note = HXD8_HUMAN.H10MO.D
WAATWTGCAYN

SEQ ID NO: 274 - protein, 10 aa, Homo sapiens
  source 1..10: note = GFI1_HUMAN.H10MO.C
GCWSTGATTT

SEQ ID NO: 275 - protein, 12 aa, Homo sapiens
  source 1..12: note = EMX1_HUMAN.H10MO.D
TAATTAGYTAAW

SEQ ID NO: 276 - protein, 18 aa, Homo sapiens
  source 1..18: note = SRF_HUMAN.H10MO.A
MTTBKCCTTMTWTGGCCW

SEQ ID NO: 277 - protein, 11 aa, Homo sapiens
  source 1..11: note = PITX1_HUMAN.H10MO.D
DGGATTAAVGG

SEQ ID NO: 278 - protein, 13 aa, Homo sapiens
  source 1..13: note = MEIS1_HUMAN.H10MO.C
TGACAGBTTWAHG

SEQ ID NO: 279 - protein, 15 aa, Homo sapiens
  source 1..15: note = TEAD3_HUMAN.H10MO.D
GAHATTYYWGCYBYW

SEQ ID NO: 280 - protein, 14 aa, Homo sapiens
  source 1..14: note = PPARD_HUMAN.H10MO.D
YAGGNBAAAGGTCA

SEQ ID NO: 281 - protein, 18 aa, Homo sapiens
  source 1..18: note = PAX2_HUMAN.H10MO.D
RHTCAGTSAYGMGTGAYW

SEQ ID NO: 282 - protein, 15 aa, Homo sapiens
  source 1..15: note = ZN423_HUMAN.H10MO.D
GRCACCCWAGGGTGC

SEQ ID NO: 283 - protein, 11 aa, Homo sapiens
  source 1..11: note = MEOX2_HUMAN.H10MO.D
TTAATKAYBWT

SEQ ID NO: 284 - protein, 10 aa, Homo sapiens
  source 1..10: note = MITF_HUMAN.H10MO.C
RWCATGTGMB

SEQ ID NO: 285 - protein, 16 aa, Homo sapiens
  source 1..16: note = SOX8_HUMAN.H10MO.D
WSACTGMAATTGATKY

SEQ ID NO: 286 - protein, 12 aa, Homo sapiens
  source 1..12: note = EOMES_HUMAN.H10MO.D
RRGAGGTGWKAA

SEQ ID NO: 287 - protein, 10 aa, Homo sapiens
  source 1..10: note = PO3F2_HUMAN.H10MO.D
AWWATTYATG

SEQ ID NO: 288 - protein, 9 aa, Homo sapiens
  source 1..9: note = HIC1_HUMAN.H10MO.C
GGGKTGCCC

SEQ ID NO: 289 - protein, 11 aa, Homo sapiens
  source 1..11: note = TBP_HUMAN.H10MO.C
RRTATAWAWDS

SEQ ID NO: 290 - DNA, 12 bp, Homo sapiens
  source 1..12: mol_type = other DNA; note = TAL1_HUMAN.H10MO.S
gaacagatggtc

SEQ ID NO: 291 - protein, 12 aa, Homo sapiens
  source 1..12: note = ZEP1_HUMAN.H10MO.D
TGGGAAATCCCB

SEQ ID NO: 292 - DNA, 14 bp, Homo sapiens
  source 1..14: mol_type = other DNA; note = PO4F1_HUMAN.H10MO.D
cattaattattcat

SEQ ID NO: 293 - protein, 11 aa, Homo sapiens
  source 1..11: note = SMRC1_HUMAN.H10MO.D
SBVTGAGTCAB

SEQ ID NO: 294 - protein, 17 aa, Homo sapiens
  source 1..17: note = NANOG_HUMAN.H10MO.A
YYWTTGWNATGCAAATK

SEQ ID NO: 295 - protein, 11 aa, Homo sapiens
  source 1..11: note = HXB13_HUMAN.H10MO.D
DDTTTTAYKRG

SEQ ID NO: 296 - protein, 11 aa, Homo sapiens
  source 1..11: note = MEIS3_HUMAN.H10MO.D
DKTGACAGSTK

SEQ ID NO: 297 - protein, 9 aa, Homo sapiens
  source 1..9: note = MAFB_HUMAN.H10MO.D
WGCTGACDS

SEQ ID NO: 298 - protein, 9 aa, Homo sapiens
  source 1..9: note = MZF1_HUMAN.H10MO.D
RGDGGGGAW

SEQ ID NO: 299 - protein, 9 aa, Homo sapiens
  source 1..9: note = GATA4_HUMAN.H10MO.B
RSWGATAAV

SEQ ID NO: 300 - protein, 12 aa, Homo sapiens
  source 1..12: note = FOXI1_HUMAN.H10MO.B
YTBTGATTGGYY

SEQ ID NO: 301 - DNA, 10 bp, Homo sapiens
  source 1..10: mol_type = other DNA; note = MAFK_HUMAN.H10MO.S
tgctgagtca

SEQ ID NO: 302 - protein, 15 aa, Homo sapiens
  source 1..15: note = GSX1_HUMAN.H10MO.D
TAATTAGNDDTTAAT

SEQ ID NO: 303 - protein, 11 aa, Homo sapiens
  source 1..11: note = GATA3_HUMAN.H10MO.C
WVAGATAAVAW

SEQ ID NO: 304 - DNA, 10 bp, Homo sapiens
  source 1..10: mol_type = other DNA; note = E2F2_HUMAN.H10MO.B
ggcgcgaaac

SEQ ID NO: 305 - protein, 11 aa, Homo sapiens
  source 1..11: note = SRBP1_HUMAN.H10MO.B
GGTGGGGTGAB

SEQ ID NO: 306 - protein, 9 aa, Homo sapiens
  source 1..9: note = NOBOX_HUMAN.H10MO.C
HTAATTRSY

SEQ ID NO: 307 - protein, 15 aa, Homo sapiens
  source 1..15: note = PROP1_HUMAN.H10MO.D
RASHAATTAAHATVR

SEQ ID NO: 308 - protein, 13 aa, Homo sapiens
  source 1..13: note = KLF14_HUMAN.H10MO.D
DRGKGGGCGTGGY

SEQ ID NO: 309 - protein, 9 aa, Homo sapiens
  source 1..9: note = NR4A2_HUMAN.H10MO.C
AAAGGTCAS

SEQ ID NO: 310 - protein, 8 aa, Homo sapiens
  source 1..8: note = MYCN_HUMAN.H10MO.B
CCACGTGS

SEQ ID NO: 311 - protein, 13 aa, Homo sapiens
  source 1..13: note = MEIS2_HUMAN.H10MO.B
TTGACAGBTGTCM

SEQ ID NO: 312 - protein, 17 aa, Homo sapiens
  source 1..17: note = THA_HUMAN.H10MO.C
RGGTCABYTSAGGTCAK

SEQ ID NO: 313 - protein, 17 aa, Homo sapiens
  source 1..17: note = PPARG_HUMAN.H10MO.A
AWBTRGGKCAAAGGKCA

SEQ ID NO: 314 - protein, 9 aa, Homo sapiens
  source 1..9: note = TFEB_HUMAN.H10MO.C
RGTCACGTG

SEQ ID NO: 315 - protein, 12 aa, Homo sapiens
  source 1..12: note = E2F8_HUMAN.H10MO.D
TTTRGMGGGAAW

SEQ ID NO: 316 - protein, 16 aa, Homo sapiens
  source 1..16: note = VAX2_HUMAN.H10MO.D
CACTAATTAVSYTWWH

SEQ ID NO: 317 - protein, 10 aa, Homo sapiens
  source 1..10: note = TFE3_HUMAN.H10MO.C
RGTCAYGTGV

SEQ ID NO: 318 - protein, 12 aa, Homo sapiens
  source 1..12: note = RFX1_HUMAN.H10MO.C
GTTGCYAGGSAA

SEQ ID NO: 319 - protein, 13 aa, Homo sapiens
  source 1..13: note = LHX2_HUMAN.H10MO.D
YRSDTTYAATWAG

SEQ ID NO: 320 - protein, 11 aa, Homo sapiens
  source 1..11: note = CEBPZ_HUMAN.H10MO.D
DCTSATTGGCT

SEQ ID NO: 321 - protein, 9 aa, Homo sapiens
  source 1..9: note = ITF2_HUMAN.H10MO.B
VCAGGTGCD

SEQ ID NO: 322 - protein, 13 aa, Homo sapiens
  source 1..13: note = SCRT2_HUMAN.H10MO.D
RWGCAACAGGTGK

SEQ ID NO: 323 - protein, 10 aa, Homo sapiens
  source 1..10: note = NFAC4_HUMAN.H10MO.C
DGGAAAAYTW

SEQ ID NO: 324 - protein, 14 aa, Homo sapiens
  source 1..14: note = ZN282_HUMAN.H10MO.D
MGKRTTRTGGGARA

SEQ ID NO: 325 - protein, 11 aa, Homo sapiens
  source 1..11: note = EGR1_HUMAN.H10MO.S
BGCGKGGGCGG

SEQ ID NO: 326 - protein, 12 aa, Homo sapiens
  source 1..12: note = EMX2_HUMAN.H10MO.D
TAATTAGYDWAW

SEQ ID NO: 327 - protein, 11 aa, Homo sapiens
  source 1..11: note = MYBA_HUMAN.H10MO.D
HRACCGTTAAM

SEQ ID NO: 328 - protein, 12 aa, Homo sapiens
  source 1..12: note = ELK3_HUMAN.H10MO.D
VMCHGGAARTSC

SEQ ID NO: 329 - protein, 22 aa, Homo sapiens
  source 1..22: note = MAFF_HUMAN.H10MO.A
AWWWHTGCTGASTCAGCAWWWW

SEQ ID NO: 330 - protein, 14 aa, Homo sapiens
  source 1..14: note = BSH_HUMAN.H10MO.D
YVATTAACBMATTA

SEQ ID NO: 331 - protein, 12 aa, Homo sapiens
  source 1..12: note = HAND1_HUMAN.H10MO.D
GBRTCTGGMWTT

SEQ ID NO: 332 - protein, 19 aa, Homo sapiens
  source 1..19: note = P63_HUMAN.H10MO.A
RRRCAWGYCTGGRCWTGYH

SEQ ID NO: 333 - protein, 12 aa, Homo sapiens
  source 1..12: note = HXA10_HUMAN.H10MO.C
KATRRTTTATGW

SEQ ID NO: 334 - skipped

SEQ ID NO: 335 - protein, 17 aa, Homo sapiens
  source 1..17: note = GBX1_HUMAN.H10MO.D
BTAATTRRYNHWWWWWW

SEQ ID NO: 336 - protein, 11 aa, Homo sapiens
  source 1..11: note = EGR3_HUMAN.H10MO.D
WGAGTGGGYGT

SEQ ID NO: 337 - protein, 25 aa, Homo sapiens
  source 1..25: note = TBX2_HUMAN.H10MO.D
TGVMWWCABAGGTGTGARRHGYSMC

SEQ ID NO: 338 - protein, 15 aa, Homo sapiens
  source 1..15: note = HXB2_HUMAN.H10MO.D
WAGAAYVTTCTAGAA

SEQ ID NO: 339 - protein, 20 aa, Homo sapiens
  source 1..20: note = BHE41_HUMAN.H10MO.D
WSVRKSYCACGTGMWGRRRV

SEQ ID NO: 340 - protein, 14 aa, Homo sapiens
  source 1..14: note = PRDM1_HUMAN.H10MO.C
RAAAGTGAAAGTGA

SEQ ID NO: 341 - protein, 17 aa, Homo sapiens
  source 1..17: note = GBX2_HUMAN.H10MO.D
BTAATTRSYBHHWWWWW

SEQ ID NO: 342 - protein, 11 aa, Homo sapiens
  source 1..11: note = ISL2_HUMAN.H10MO.D
YVYTAAKTGCW

SEQ ID NO: 343 - protein, 19 aa, Homo sapiens
  source 1..19: note = LMX1A_HUMAN.H10MO.D
TAATTAATTWKKTWATTWW

SEQ ID NO: 344 - protein, 14 aa, Homo sapiens
  source 1..14: note = ZN740_HUMAN.H10MO.D
DKKGGGGGGGHRDK

SEQ ID NO: 345 - protein, 11 aa, Homo sapiens
  source 1..11: note = ZN652_HUMAN.H10MO.D
AARGGGTTAAW

SEQ ID NO: 346 - protein, 13 aa, Homo sapiens
  source 1..13: note = PKNX2_HUMAN.H10MO.D
DKGRCRGGTGKSA

SEQ ID NO: 347 - protein, 11 aa, Homo sapiens
  source 1..11: note = OLIG3_HUMAN.H10MO.D
ACCATATGKTB

SEQ ID NO: 348 - protein, 8 aa, Homo sapiens
  source 1..8: note = ARNT_HUMAN.H10MO.B
BYRCGTGC

SEQ ID NO: 349 - protein, 12 aa, Homo sapiens
  source 1..12: note = ZN384_HUMAN.H10MO.C
GSAAAAAVYMKS

SEQ ID NO: 350 - protein, 13 aa, Homo sapiens
  source 1..13: note = HXA11_HUMAN.H10MO.D
TWAADTTTTAYGR

SEQ ID NO: 351 - protein, 18 aa, Homo sapiens
  source 1..18: note = LHX8_HUMAN.H10MO.D
YRYTRATTRCMATTAATT

SEQ ID NO: 352 - protein, 11 aa, Homo sapiens
  source 1..11: note = DBP_HUMAN.H10MO.B
VTTATGYAABM

SEQ ID NO: 353 - protein, 10 aa, Homo sapiens
  source 1..10: note = E2F5_HUMAN.H10MO.B
SGCGCSAAAH

SEQ ID NO: 354 - protein, 19 aa, Homo sapiens
  source 1..19: note = GATA1_HUMAN.H10MO.A
CTGBNNVVBRSAGATAAGR

SEQ ID NO: 355 - protein, 22 aa, Homo sapiens
  source 1..22: note = STAT1_HUMAN.H10MO.A
YCMTTTCCHGGAAAWCHCATKM

SEQ ID NO: 356 - protein, 18 aa, Homo sapiens
  source 1..18: note = LHX6_HUMAN.H10MO.D
VYTAATTAGYRYTAATTA

SEQ ID NO: 357 - protein, 7 aa, Homo sapiens
  source 1..7: note = SOX15_HUMAN.H10MO.D
CWTTGTT

SEQ ID NO: 358 - protein, 12 aa, Homo sapiens
  source 1..12: note = RELB_HUMAN.H10MO.C
RGGGRMTTTCCM

SEQ ID NO: 359 - protein, 11 aa, Homo sapiens
  source 1..11: note = KLF13_HUMAN.H10MO.D
DRGKGGGCGKD

SEQ ID NO: 360 - protein, 11 aa, Homo sapiens
  source 1..11: note = GMEB2_HUMAN.H10MO.D
NBKTACGTVRN

SEQ ID NO: 361 - protein, 11 aa, Homo sapiens
  source 1..11: note = ZN784_HUMAN.H10MO.D
DMGGTAGGTRV

SEQ ID NO: 362 - protein, 16 aa, Homo sapiens
  source 1..16: note = MNT_HUMAN.H10MO.D
RKRRRDKVAGGKGDDR

SEQ ID NO: 363 - protein, 13 aa, Homo sapiens
  source 1..13: note = SRBP2_HUMAN.H10MO.B
VSGTGGGGWGABG

SEQ ID NO: 364 - protein, 11 aa, Homo sapiens
  source 1..11: note = FOXF2_HUMAN.H10MO.D
TGTTTACHTWD

SEQ ID NO: 365 - protein, 16 aa, Homo sapiens
  source 1..16: note = DLX4_HUMAN.H10MO.D
TAATTASNBHWWWWWD

SEQ ID NO: 366 - protein, 7 aa, Homo sapiens
  source 1..7: note = SOX10_HUMAN.H10MO.D
BCWTTGT

SEQ ID NO: 367 - protein, 16 aa, Homo sapiens
  source 1..16: note = GSX2_HUMAN.H10MO.D
YTAATTRSHWWTTAAT

SEQ ID NO: 368 - DNA, 13 bp, Homo sapiens
  source 1..13: mol_type = other DNA; note = CR3L2_HUMAN.H10MO.D
tgatgacgtggca

SEQ ID NO: 369 - protein, 20 aa, Homo sapiens
  source 1..20: note = ZSC16_HUMAN.H10MO.D
RRGGTGYTCTGTTAACAMTW

SEQ ID NO: 370 - protein, 13 aa, Homo sapiens
  source 1..13: note = BACH1_HUMAN.H10MO.A
TGCTGAGTCABSS

SEQ ID NO: 371 - protein, 14 aa, Homo sapiens
  source 1..14: note = PO3F1_HUMAN.H10MO.C
VATKSWWATGCAAW

SEQ ID NO: 372 - protein, 13 aa, Homo sapiens
  source 1..13: note = CREB3_HUMAN.H10MO.D
KGRTGACGTGGCA

SEQ ID NO: 373 - protein, 13 aa, Homo sapiens
  source 1..13: note = ZBTB6_HUMAN.H10MO.D
VGRTGATRGAGCC

SEQ ID NO: 374 - protein, 13 aa, Homo sapiens
  source 1..13: note = NFYB_HUMAN.H10MO.A
YRRCCAATSAGMR

SEQ ID NO: 375 - protein, 17 aa, Homo sapiens
  source 1..17: note = ANDR_HUMAN.H10MO.A
DGBACABHCTGTTCYHK

SEQ ID NO: 376 - protein, 11 aa, Homo sapiens
  source 1..11: note = MAX_HUMAN.H10MO.A
RGYCACGTGST

SEQ ID NO: 377 - protein, 9 aa, Homo sapiens
  source 1..9: note = PDX1_HUMAN.H10MO.C
RGTAATTAG

SEQ ID NO: 378 - protein, 20 aa, Homo sapiens
  source 1..20: note = TBX4_HUMAN.H10MO.D
RAGGTGTGAWAWNTVACACC

SEQ ID NO: 379 - protein, 13 aa, Homo sapiens
  source 1..13: note = BARH1_HUMAN.H10MO.D
TAAWYGBYBHTAA

SEQ ID NO: 380 - protein, 14 aa, Homo sapiens
  source 1..14: note = TEF_HUMAN.H10MO.D
TGTTTATGTAAMTK

SEQ ID NO: 381 - protein, 10 aa, Homo sapiens
  source 1..10: note = RXRB_HUMAN.H10MO.C
TSAGGTCASR

SEQ ID NO: 382 - protein, 11 aa, Homo sapiens
  source 1..11: note = NDF2_HUMAN.H10MO.D
DBSRTATGDTS

SEQ ID NO: 383 - protein, 7 aa, Homo sapiens
  source 1..7: note = MECP2_HUMAN.H10MO.C
SCCGGRR

SEQ ID NO: 384 - protein, 11 aa, Homo sapiens
  source 1..11: note = ZBT49_HUMAN.H10MO.D
NBSNTAATTRV

SEQ ID NO: 385 - protein, 11 aa, Homo sapiens
  source 1..11: note = HIC2_HUMAN.H10MO.D
HCRTGCCAGVN

SEQ ID NO: 386 - protein, 8 aa, Homo sapiens
  source 1..8: note = CDX2_HUMAN.H10MO.C
WTTTATRD

SEQ ID NO: 387 - protein, 12 aa, Homo sapiens
  source 1..12: note = FOXQ1_HUMAN.H10MO.C
HATTGTTTATTT

SEQ ID NO: 388 - protein, 17 aa, Homo sapiens
  source 1..17: note = RARA_HUMAN.H10MO.C
RGGTCANSRRVAGGTCA

SEQ ID NO: 389 - protein, 9 aa, Homo sapiens
  source 1..9: note = NFAC2_HUMAN.H10MO.B
TGGAAAAWY

SEQ ID NO: 390 - protein, 19 aa, Homo sapiens
  source 1..19: note = ZN232_HUMAN.H10MO.D
TGTTAAAYGTAGATTAAGY

SEQ ID NO: 391 - protein, 13 aa, Homo sapiens
  source 1..13: note = FOXA1_HUMAN.H10MO.A
WTRTTTACWYWDB

SEQ ID NO: 392 - protein, 11 aa, Homo sapiens
  source 1..11: note = HME2_HUMAN.H10MO.D
NNTRRTTARNB

SEQ ID NO: 393 - protein, 16 aa, Homo sapiens
  source 1..16: note = FOXO1_HUMAN.H10MO.C
DDBTTGTTTWBWWTTT

SEQ ID NO: 394 - protein, 18 aa, Homo sapiens
  source 1..18: note = LHX9_HUMAN.H10MO.D
YTAATTAGYNHWWWWWWD

SEQ ID NO: 395 - protein, 20 aa, Homo sapiens
  source 1..20: note = DMBX1_HUMAN.H10MO.D
YMMTWWWAAYCYAATTAABD

SEQ ID NO: 396 - protein, 10 aa, Homo sapiens
  source 1..10: note = HXA5_HUMAN.H10MO.D
YTRATTAATG

SEQ ID NO: 397 - protein, 12 aa, Homo sapiens
  source 1..12: note = JDP2_HUMAN.H10MO.D
BGATGACGTCAY

SEQ ID NO: 398 - protein, 11 aa, Homo sapiens
  source 1..11: note = ATOH1_HUMAN.H10MO.D
YRMCATATGYY

SEQ ID NO: 399 - protein, 10 aa, Homo sapiens
  source 1..10: note = PITX2_HUMAN.H10MO.D
TKGGATTAVH

SEQ ID NO: 400 - protein, 11 aa, Homo sapiens
  source 1..11: note = PAX7_HUMAN.H10MO.D
TAATYGATTAW

SEQ ID NO: 401 - protein, 9 aa, Homo sapiens
  source 1..9: note = NKX28_HUMAN.H10MO.C
TTCAAGKRC

SEQ ID NO: 402 - protein, 8 aa, Homo sapiens
  source 1..8: note = NR1I2_HUMAN.H10MO.S
RAGTTCAB

SEQ ID NO: 403 - protein, 11 aa, Homo sapiens
  source 1..11: note = HXA13_HUMAN.H10MO.C
GDTTTTATTGG

SEQ ID NO: 404 - protein, 14 aa, Homo sapiens
  source 1..14: note = NFE2_HUMAN.H10MO.B
RSVATGACTCAGCA

SEQ ID NO: 405 - protein, 10 aa, Homo sapiens
  source 1..10: note = DLX3_HUMAN.H10MO.C
GMTAATTRSW

SEQ ID NO: 406 - protein, 11 aa, Homo sapiens
  source 1..11: note = IRX3_HUMAN.H10MO.D
WNWCATGTWMT

SEQ ID NO: 407 - protein, 14 aa, Homo sapiens
  source 1..14: note = HNF6_HUMAN.H10MO.C
WWTATTGATTTWDH

SEQ ID NO: 408 - protein, 20 aa, Homo sapiens
  source 1..20: note = ZN639_HUMAN.H10MO.D
SRVKGCGCATGCGCAGVRVS

SEQ ID NO: 409 - protein, 15 aa, Homo sapiens
  source 1..15: note = KLF3_HUMAN.H10MO.D
HRCYWGGGTGKGGCT

SEQ ID NO: 410 - protein, 13 aa, Homo sapiens
  source 1..13: note = PPARA_HUMAN.H10MO.C
RGGTCARAGGTCA

SEQ ID NO: 411 - protein, 16 aa, Homo sapiens
  source 1..16: note = PO5F1_HUMAN.H10MO.A
CYWTTSWTATGCAAAT

SEQ ID NO: 412 - protein, 14 aa, Homo sapiens
  source 1..14: note = TFDP1_HUMAN.H10MO.S
AWWKRGCGGGAAAY

SEQ ID NO: 413 - protein, 13 aa, Homo sapiens
  source 1..13: note = TEAD4_HUMAN.H10MO.A
RCATTCCWGVSMT

SEQ ID NO: 414 - protein, 11 aa, Homo sapiens
  source 1..11: note = RUNX1_HUMAN.H10MO.A
BYTGTGGTTWB

SEQ ID NO: 415 - protein, 19 aa, Homo sapiens
  source 1..19: note = ZN713_HUMAN.H10MO.D
RWAGAARAADGVSANGMAA

SEQ ID NO: 416 - protein, 12 aa, Homo sapiens
  source 1..12: note = HIF1A_HUMAN.H10MO.A
SBSTACGTGCSB

SEQ ID NO: 417 - protein, 9 aa, Homo sapiens
  source 1..9: note = AHR_HUMAN.H10MO.B
DTYGCGTGM

SEQ ID NO: 418 - protein, 11 aa, Homo sapiens
  source 1..11: note = NKX23_HUMAN.H10MO.D
BTYAAGTGGKW

SEQ ID NO: 419 - protein, 7 aa, Homo sapiens
  source 1..7: note = RARG_HUMAN.H10MO.S
RAGGTCA

SEQ ID NO: 420 - protein, 17 aa, Homo sapiens
  source 1..17: note = EHF_HUMAN.H10MO.C
RRAVVAGGAARTRRRRR

SEQ ID NO: 421 - DNA, 17 bp, Homo sapiens
  source 1..17: mol_type = other DNA; note = TBX20_HUMAN.H10MO.D
aggtgtgaaggtgtgaa

SEQ ID NO: 422 - protein, 16 aa, Homo sapiens
  source 1..16: note = P5F1B_HUMAN.H10MO.D
DMATTTGCATATTYAW

SEQ ID NO: 423 - protein, 14 aa, Homo sapiens
  source 1..14: note = HSF2_HUMAN.H10MO.A
VGAABRTTCTAGAA

SEQ ID NO: 424 - protein, 18 aa, Homo sapiens
  source 1..18: note = BARH2_HUMAN.H10MO.D
VYTAAWYGNTSHWWWWHR

SEQ ID NO: 425 - protein, 11 aa, Homo sapiens
  source 1..11: note = MYC_HUMAN.H10MO.A
GASCACGTGGY

SEQ ID NO: 426 - protein, 15 aa, Homo sapiens
  source 1..15: note = RFX4_HUMAN.H10MO.D
SGTTGCCATGGYWAC

SEQ ID NO: 427 - protein, 15 aa, Homo sapiens
  source 1..15: note = TF2LX_HUMAN.H10MO.D
GACAGCTGTCATWRY

SEQ ID NO: 428 - protein, 14 aa, Homo sapiens
  source 1..14: note = PRD14_HUMAN.H10MO.C
RRGTTAGRGACCYR

SEQ ID NO: 429 - protein, 12 aa, Homo sapiens
  source 1..12: note = MEF2D_HUMAN.H10MO.C
GCTATTTWTAGV

SEQ ID NO: 430 - protein, 17 aa, Homo sapiens
  source 1..17: note = SHOX_HUMAN.H10MO.D
YTAATTRRYTWWWWWWD

SEQ ID NO: 431 - protein, 11 aa, Homo sapiens
  source 1..11: note = NKX62_HUMAN.H10MO.D
NDTTAATKASN

SEQ ID NO: 432 - protein, 17 aa, Homo sapiens
  source 1..17: note = PO3F4_HUMAN.H10MO.D
TMATTTGCATAATTWAW

SEQ ID NO: 433 - protein, 20 aa, Homo sapiens
  source 1..20: note = IRF1_HUMAN.H10MO.A
RAAASNGAAAGTGAAASTRR

SEQ ID NO: 434 - protein, 11 aa, Homo sapiens
  source 1..11: note = DRGX_HUMAN.H10MO.D
TAATYTAATTA

SEQ ID NO: 435 - protein, 14 aa, Homo sapiens
  source 1..14: note = VSX1_HUMAN.H10MO.D
TAATTAGCHWWWWW

SEQ ID NO: 436 - protein, 13 aa, Homo sapiens
  source 1..13: note = ZBED1_HUMAN.H10MO.D
YATGTCGCGAYAK

SEQ ID NO: 437 - protein, 12 aa, Homo sapiens
  source 1..12: note = XBP1_HUMAN.H10MO.C
GACGTGKCMTWW

SEQ ID NO: 438 - protein, 14 aa, Homo sapiens
  source 1..14: note = NR1D1_HUMAN.H10MO.C
AWAARTAGGTCARW

SEQ ID NO: 439 - protein, 7 aa, Homo sapiens
  source 1..7: note = NKX25_HUMAN.H10MO.C
TYAAGTG

SEQ ID NO: 440 - protein, 16 aa, Homo sapiens
  source 1..16: note = ZN589_HUMAN.H10MO.D
CCMASGGKWWYWRCCS

SEQ ID NO: 441 - protein, 10 aa, Homo sapiens
  source 1..10: note = SMAD3_HUMAN.H10MO.C
STGTCTGKCY

SEQ ID NO: 442 - protein, 13 aa, Homo sapiens
  source 1..13: note = MEF2C_HUMAN.H10MO.C
DKCTATTTTTRGM

SEQ ID NO: 443 - protein, 22 aa, Homo sapiens
  source 1..22: note = RFX2_HUMAN.H10MO.C
SSSSCSGTTGCCATGGMRACSG

SEQ ID NO: 444 - protein, 11 aa, Homo sapiens
  source 1..11: note = HMBX1_HUMAN.H10MO.D
DVCTRGTTAMN

SEQ ID NO: 445 - protein, 15 aa, Homo sapiens
  source 1..15: note = OTX2_HUMAN.H10MO.C
KHDMWVGGATTARRK

SEQ ID NO: 446 - protein, 13 aa, synthetic construct
  source 1..13: note = RXRG_HUMAN.H10MO.B
GGTCAAAGGTCAH

SEQ ID NO: 447 - protein, 12 aa, Homo sapiens
  source 1..12: note = CEBPB_HUMAN.H10MO.A
KATTGCRYAAYH

SEQ ID NO: 448 - protein, 15 aa, Homo sapiens
  source 1..15: note = HMGA2_HUMAN.H10MO.D
AATWWYSSSSAATAT

SEQ ID NO: 449 - protein, 22 aa, Homo sapiens
  source 1..22: note = MAFK_HUMAN.H10MO.A
RAAWNTGCTGASTCAGCAWWWW

SEQ ID NO: 450 - protein, 8 aa, Homo sapiens
  source 1..8: note = HXD4_HUMAN.H10MO.D
TTAATTGW

SEQ ID NO: 451 - protein, 11 aa, Homo sapiens
  source 1..11: note = CREB5_HUMAN.H10MO.D
RRTGACGTMAT

SEQ ID NO: 452 - protein, 11 aa, Homo sapiens
  source 1..11: note = RX_HUMAN.H10MO.D
TTAATTRRYWA

SEQ ID NO: 453 - protein, 13 aa, Homo sapiens
  source 1..13: note = ENOA_HUMAN.H10MO.A
SRSCAYGTGKSHR

SEQ ID NO: 454 - protein, 13 aa, Homo sapiens
  source 1..13: note = PRGR_HUMAN.H10MO.C
RGAACANYCTGTH

SEQ ID NO: 455 - protein, 10 aa, Homo sapiens
  source 1..10: note = NR5A2_HUMAN.H10MO.C
YYCAAGGYYA

SEQ ID NO: 456 - protein, 9 aa, Homo sapiens
  source 1..9: note = FOXP2_HUMAN.H10MO.A
BTGTTTACN

SEQ ID NO: 457 - protein, 21 aa, Homo sapiens
  source 1..21: note = ONEC2_HUMAN.H10MO.D
DKSWTKWTATKGATTTTTYYT

SEQ ID NO: 458 - protein, 18 aa, Homo sapiens
  source 1..18: note = LMX1B_HUMAN.H10MO.D
TAATTAAHWVMYTWWWWW

SEQ ID NO: 459 - protein, 12 aa, Homo sapiens
  source 1..12: note = HEY1_HUMAN.H10MO.D
KGRCACGTGBCK

SEQ ID NO: 460 - protein, 19 aa, Homo sapiens
  source 1..19: note = NKX61_HUMAN.H10MO.D
WWTTAATKASBTTTWAWTD

SEQ ID NO: 461 - protein, 11 aa, Homo sapiens
  source 1..11: note = DPRX_HUMAN.H10MO.D
VSGGATTADCB

SEQ ID NO: 462 - protein, 13 aa, Homo sapiens
  source 1..13: note = HXB6_HUMAN.H10MO.D
AWTGATTKATGMM

SEQ ID NO: 463 - protein, 11 aa, Homo sapiens
  source 1..11: note = HXA2_HUMAN.H10MO.D
NGTAATTARBD

SEQ ID NO: 464 - protein, 15 aa, Homo sapiens
  source 1..15: note = PBX2_HUMAN.H10MO.C
KRAWKTGATTGATKS

SEQ ID NO: 465 - protein, 14 aa, Homo sapiens
  source 1..14: note = RFX3_HUMAN.H10MO.B
BGTTRCCATGGYRA

SEQ ID NO: 466 - protein, 15 aa, Homo sapiens
  source 1..15: note = PO4F3_HUMAN.H10MO.D
TCATTAATTATKCAT

SEQ ID NO: 467 - protein, 7 aa, Homo sapiens
  source 1..7: note = PRRX2_HUMAN.H10MO.C
YTAATTR

SEQ ID NO: 468 - protein, 14 aa, Homo sapiens
  source 1..14: note = ZBT18_HUMAN.H10MO.D
TCCAGATGTTBVSS

SEQ ID NO: 469 - protein, 16 aa, Homo sapiens
  source 1..16: note = TF7L1_HUMAN.H10MO.D
WDRDDMWWCGGGGMGV

SEQ ID NO: 470 - protein, 12 aa, Homo sapiens
  source 1..12: note = SPIB_HUMAN.H10MO.B
ARAAASMGGAAS

SEQ ID NO: 471 - protein, 17 aa, Homo sapiens
  source 1..17: note = PAX1_HUMAN.H10MO.D
YSGTCACGCWTGANTGV

SEQ ID NO: 472 - protein, 11 aa, Homo sapiens
  source 1..11: note = PHX2A_HUMAN.H10MO.D
TAATYTAATTA

SEQ ID NO: 473 - protein, 17 aa, Homo sapiens
  source 1..17: note = HME1_HUMAN.H10MO.D
VCTAATTRSYHAWWWWW

SEQ ID NO: 474 - protein, 10 aa, Homo sapiens
  source 1..10: note = FEV_HUMAN.H10MO.C
CAGGAARTDA

SEQ ID NO: 475 - protein, 9 aa, Homo sapiens
  source 1..9: note = OVOL1_HUMAN.H10MO.C
KGTAACKGT

SEQ ID NO: 476 - protein, 12 aa, Homo sapiens
  source 1..12: note = SOX4_HUMAN.H10MO.C
CSCTTTGTTCTC

SEQ ID NO: 477 - protein, 11 aa, Homo sapiens
  source 1..11: note = VSX2_HUMAN.H10MO.D
TAATTAGCTHW

SEQ ID NO: 478 - protein, 13 aa, Homo sapiens
  source 1..13: note = CEBPA_HUMAN.H10MO.A
DRTTGTGCAAYHW

SEQ ID NO: 479 - protein, 12 aa, Homo sapiens
  source 1..12: note = HXC12_HUMAN.H10MO.D
DTTTTAYKACYT

SEQ ID NO: 480 - DNA, 17 bp, Homo sapiens
  source 1..17: mol_type = other DNA; note = ZBTB4_HUMAN.H10MO.D
cctagatggcaggtggg

SEQ ID NO: 481 - protein, 7 aa, Homo sapiens
  source 1..7: note = CXXC1_HUMAN.H10MO.D
CGKTGKY

SEQ ID NO: 482 - protein, 17 aa, Homo sapiens
  source 1..17: note = NFIC_HUMAN.H10MO.A
HYYTGGCWSNVDKCCMR

SEQ ID NO: 483 - protein, 11 aa, Homo sapiens
  source 1..11: note = PEBB_HUMAN.H10MO.C
TYTGTGGTYWB

SEQ ID NO: 484 - protein, 11 aa, Homo sapiens
  source 1..11: note = CEBPD_HUMAN.H10MO.B
RRTKDBGCAAT

SEQ ID NO: 485 - protein, 17 aa, Homo sapiens
  source 1..17: note = SHOX2_HUMAN.H10MO.D
YTAATTRRYYHWWWWWD

SEQ ID NO: 486 - protein, 19 aa, Homo sapiens
  source 1..19: note = MNX1_HUMAN.H10MO.D
MBATTTDHWTHWCYHYTHH

SEQ ID NO: 487 - protein, 18 aa, Homo sapiens
  source 1..18: note = SOX17_HUMAN.H10MO.D
CAAMAATNHHCATTGTCS

SEQ ID NO: 488 - protein, 11 aa, Homo sapiens
  source 1..11: note = JUNB_HUMAN.H10MO.C
GBVTGAGTCAY

SEQ ID NO: 489 - protein, 7 aa, Homo sapiens
  source 1..7: note = LEF1_HUMAN.H10MO.C
CTTTGWW

SEQ ID NO: 490 - protein, 11 aa, Homo sapiens
  source 1..11: note = ELK4_HUMAN.H10MO.A
ACCGGAAGTRN

SEQ ID NO: 491 - protein, 14 aa, Homo sapiens
  source 1..14: note = ASCL2_HUMAN.H10MO.D
KKGRGYRGSTGGKK

SEQ ID NO: 492 - protein, 14 aa, Homo sapiens
  source 1..14: note = HNF1B_HUMAN.H10MO.B
RGTTAATRWTTAAC

SEQ ID NO: 493 - protein, 11 aa, Homo sapiens
  source 1..11: note = FOXA2_HUMAN.H10MO.A
TGTTTACWYWG

SEQ ID NO: 494 - protein, 18 aa, Homo sapiens
  source 1..18: note = NR2F6_HUMAN.H10MO.D
RGGWCAAAGKTCANNKRR

SEQ ID NO: 495 - protein, 17 aa, Homo sapiens
  source 1..17: note = BC11A_HUMAN.H10MO.C
RAAAGAGGAAGTGARAV

SEQ ID NO: 496 - protein, 11 aa, Homo sapiens
  source 1..11: note = HXD13_HUMAN.H10MO.D
TTTATTAGRGA

SEQ ID NO: 497 - protein, 9 aa, Homo sapiens
  source 1..9: note = ZN333_HUMAN.H10MO.D
SKATAATGA

SEQ ID NO: 498 - protein, 17 aa, Homo sapiens
  source 1..17: note = SPI1_HUMAN.H10MO.A
RRAAAGAGGAAGTGARA

SEQ ID NO: 499 - protein, 10 aa, Homo sapiens
  source 1..10: note = HXD9_HUMAN.H10MO.D
ARWTTNATKD

SEQ ID NO: 500 - protein, 8 aa, Homo sapiens
  source 1..8: note = NR1I3_HUMAN.H10MO.S
RAGKTCAB

SEQ ID NO: 501 - protein, 13 aa, Homo sapiens
  source 1..13: note = CEBPG_HUMAN.H10MO.C
ATTDTGCAATCTB

SEQ ID NO: 502 - protein, 9 aa, Homo sapiens
  source 1..9: note = NR4A1_HUMAN.H10MO.C
VAAGGTCAS

SEQ ID NO: 503 - protein, 12 aa, Homo sapiens
  source 1..12: note = ONEC3_HUMAN.H10MO.D
TATYGATTTTTT

SEQ ID NO: 504 - protein, 12 aa, Homo sapiens
  source 1..12: note = SMAD2_HUMAN.H10MO.C
TGTCTGDCACCT

SEQ ID NO: 505 - protein, 10 aa, Homo sapiens
  source 1..10: note = GATA1_HUMAN.H10MO.S
DSAGATAAGR

SEQ ID NO: 506 - protein, 17 aa, Homo sapiens
  source 1..17: note = IRF3_HUMAN.H10MO.C
GGAAAGBGAAASBRRRA

SEQ ID NO: 507 - protein, 12 aa, Homo sapiens
  source 1..12: note = HES5_HUMAN.H10MO.D
YGGCACGTGYCR

SEQ ID NO: 508 - DNA, 14 bp, Homo sapiens
  source 1..14: mol_type = other DNA; note = NR2E3_HUMAN.H10MO.C
aaagtcaaaagtca

SEQ ID NO: 509 - protein, 11 aa, Homo sapiens
  source 1..11: note = PAX6_HUMAN.H10MO.D
TSAWGCGTRAA

SEQ ID NO: 510 - protein, 16 aa, Homo sapiens
  source 1..16: note = SOX1_HUMAN.H10MO.D
DATSAATGTTATTSAT

SEQ ID NO: 511 - protein, 13 aa, Homo sapiens
  source 1..13: note = PIT1_HUMAN.H10MO.C
YTMATGAATATWY

SEQ ID NO: 512 - protein, 11 aa, Homo sapiens
  source 1..11: note = PHX2B_HUMAN.H10MO.D
TAATYTAATTA

SEQ ID NO: 513 - protein, 13 aa, Homo sapiens
  source 1..13: note = ARI5B_HUMAN.H10MO.C
DBYKDGTATTSKR

SEQ ID NO: 514 - protein, 13 aa, Homo sapiens
  source 1..13: note = PKNX1_HUMAN.H10MO.D
VRVTTGAYTGATK

SEQ ID NO: 515 - protein, 11 aa, Homo sapiens
  source 1..11: note = TFAP4_HUMAN.H10MO.C
RYCAGCTGYGG

SEQ ID NO: 516 - protein, 12 aa, Homo sapiens
  source 1..12: note = NKX22_HUMAN.H10MO.D
KTYAAGTRSTTD

SEQ ID NO: 517 - protein, 16 aa, Homo sapiens
  source 1..16: note = ISX_HUMAN.H10MO.D
YTAATTRGHTWWWWWW

SEQ ID NO: 518 - protein, 11 aa, Homo sapiens
  source 1..11: note = ETV2_HUMAN.H10MO.D
YNYKRTTTYND

SEQ ID NO: 519 - protein, 11 aa, Homo sapiens
  source 1..11: note = GCM2_HUMAN.H10MO.D
BATGCGGGTAB

SEQ ID NO: 520 - protein, 15 aa, Homo sapiens
  source 1..15: note = COT2_HUMAN.H10MO.S
VAGGTCAARGGTCAM

SEQ ID NO: 521 - protein, 18 aa, Homo sapiens
  source 1..18: note = VAX1_HUMAN.H10MO.D
RSTAATTAGBSTWAWTWW

SEQ ID NO: 522 - protein, 11 aa, Homo sapiens
  source 1..11: note = MLX_HUMAN.H10MO.D
YGATCACGTGN

SEQ ID NO: 523 - protein, 7 aa, Homo sapiens
  source 1..7: note = RARA_HUMAN.H10MO.S
SAGGTCA

SEQ ID NO: 524 - protein, 13 aa, Homo sapiens
  source 1..13: note = MYOG_HUMAN.H10MO.D
TRACAGCTGCWGC

SEQ ID NO: 525 - protein, 18 aa, Homo sapiens
  source 1..18: note = MSX1_HUMAN.H10MO.D
TAATTGSWWKTTAATTGS

SEQ ID NO: 526 - protein, 12 aa, Homo sapiens
  source 1..12: note = HLTF_HUMAN.H10MO.D
KANKGCTGSMAM

SEQ ID NO: 527 - protein, 12 aa, Homo sapiens
  source 1..12: note = BPTF_HUMAN.H10MO.D
KKTBTTGTKKKS

SEQ ID NO: 528 - protein, 19 aa, Homo sapiens
  source 1..19: note = FOXD2_HUMAN.H10MO.D
WAWGTAAATRTTWWYTTAA

SEQ ID NO: 529 - protein, 9 aa, Homo sapiens
  source 1..9: note = BRCA1_HUMAN.H10MO.D
BKTSKGTTG

SEQ ID NO: 530 - protein, 9 aa, Homo sapiens
  source 1..9: note = FOXP3_HUMAN.H10MO.D
AATKTGWTT

SEQ ID NO: 531 - protein, 11 aa, Homo sapiens
  source 1..11: note = GSC_HUMAN.H10MO.D
ADSGGATTARB

SEQ ID NO: 532 - protein, 14 aa, Homo sapiens
  source 1..14: note = HNF4A_HUMAN.H10MO.A
RRGKBCAAAGTYCA

SEQ ID NO: 533 - protein, 17 aa, Homo sapiens
  source 1..17: note = PO3F3_HUMAN.H10MO.D
WWWAWTTATGCWWATTW

SEQ ID NO: 534 - protein, 11 aa, Homo sapiens
  source 1..11: note = NKX32_HUMAN.H10MO.C
AGYTAAGTGGV

SEQ ID NO: 535 - protein, 12 aa, Homo sapiens
  source 1..12: note = BATF3_HUMAN.H10MO.D
HHTYRYTTACGK

SEQ ID NO: 536 - DNA, 11 bp, Homo sapiens
  source 1..11: mol_type = other DNA; note = UNC4_HUMAN.H10MO.D
taatttaatta

SEQ ID NO: 537 - protein, 13 aa, Homo sapiens
  source 1..13: note = FOXA3_HUMAN.H10MO.C
HTRTTTRYTTWBY

SEQ ID NO: 538 - protein, 11 aa, Homo sapiens
  source 1..11: note = HXD3_HUMAN.H10MO.D
NGKTAATTAGK

SEQ ID NO: 539 - protein, 7 aa, Homo sapiens
  source 1..7: note = NFIA_HUMAN.H10MO.S
MTTGGCW

SEQ ID NO: 540 - protein, 16 aa, Homo sapiens
  source 1..16: note = HINFP_HUMAN.H10MO.C
DMSHHMGCGGACGTTV

SEQ ID NO: 541 - protein, 19 aa, Homo sapiens
  source 1..19: note = VENTX_HUMAN.H10MO.D
YVYTAATYGSTTHHHWWTT

SEQ ID NO: 542 - protein, 12 aa, Homo sapiens
  source 1..12: note = TCF7_HUMAN.H10MO.C
ASWWCAAAGSRB

SEQ ID NO: 543 - protein, 11 aa, Homo sapiens
  source 1..11: note = LBX2_HUMAN.H10MO.D
RYTAATTRGBY

SEQ ID NO: 544 - protein, 18 aa, Homo sapiens
  source 1..18: note = PITX3_HUMAN.H10MO.D
RGGGDWTWAAGGGATTAR

SEQ ID NO: 545 - protein, 13 aa, Homo sapiens
  source 1..13: note = HES1_HUMAN.H10MO.D
VMGVCACGMGMCM

SEQ ID NO: 546 - DNA, 15 bp, Homo sapiens
  source 1..15: mol_type = other DNA; note = HXC6_HUMAN.H10MO.D
atgatttattacttt

SEQ ID NO: 547 - protein, 18 aa, Homo sapiens
  source 1..18: note = BATF_HUMAN.H10MO.A
ABTYYYRDWATGASTCAK

SEQ ID NO: 548 - protein, 21 aa, Homo sapiens
  source 1..21: note = ZBT7B_HUMAN.H10MO.D
SSCSGTTGCCATGGVRACSGS

SEQ ID NO: 549 - protein, 18 aa, Homo sapiens
  source 1..18: note = MUSC_HUMAN.H10MO.D
CAGAAGACGGCATACGAB

SEQ ID NO: 550 - protein, 17 aa, Homo sapiens
  source 1..17: note = ESX1_HUMAN.H10MO.D
RYTAATTRGYNHWWWTW

SEQ ID NO: 551 - protein, 13 aa, Homo sapiens
  source 1..13: note = FOXM1_HUMAN.H10MO.D
BTTGWKTGWTTWY

SEQ ID NO: 552 - protein, 12 aa, Homo sapiens
  source 1..12: note = HSFY1_HUMAN.H10MO.D
WWTCGAACGWWW

SEQ ID NO: 553 - protein, 13 aa, Homo sapiens
  source 1..13: note = E2F7_HUMAN.H10MO.D
WWDGGCGCGAAAM

SEQ ID NO: 554 - protein, 7 aa, Homo sapiens
  source 1..7: note = MYB_HUMAN.H10MO.C
CAGTTRD

SEQ ID NO: 555 - skipped

SEQ ID NO: 556 - protein, 17 aa, Homo sapiens
  source 1..17: note = MTF1_HUMAN.H10MO.C
MGKGCCGTGTGCAAARS

SEQ ID NO: 557 - protein, 13 aa, Homo sapiens
  source 1..13: note = FOXO3_HUMAN.H10MO.B
KRYTTGTTTWCMK

SEQ ID NO: 558 - protein, 22 aa, Homo sapiens
  source 1..22: note = ARI3A_HUMAN.H10MO.D
RATTAAWBRRWATCAAAWTWWA

SEQ ID NO: 559 - protein, 10 aa, Homo sapiens
  source 1..10: note = THA_HUMAN.H10MO.S
BTSAGGTCAS

SEQ ID NO: 560 - protein, 8 aa, Homo sapiens
  source 1..8: note = MAF_HUMAN.H10MO.B
KTGCTGAC

SEQ ID NO: 561 - protein, 7 aa, Homo sapiens
  source 1..7: note = NF2L1_HUMAN.H10MO.C
NATGACD

SEQ ID NO: 562 - protein, 9 aa, Homo sapiens
  source 1..9: note = STF1_HUMAN.H10MO.B
YCAAGGYCA

SEQ ID NO: 563 - protein, 11 aa, Homo sapiens
  source 1..11: note = P63_HUMAN.H10MO.S
GRRCWTGTCYS

SEQ ID NO: 564 - protein, 9 aa, Homo sapiens
  source 1..9: note = SRY_HUMAN.H10MO.B
YWTTGTTTH

SEQ ID NO: 565 - protein, 14 aa, Homo sapiens
  source 1..14: note = GCM1_HUMAN.H10MO.D
HWVATGCKGGYMBK

SEQ ID NO: 566 - protein, 13 aa, Homo sapiens
  source 1..13: note = HLF_HUMAN.H10MO.C
GRTTRYGTAAYMS

SEQ ID NO: 567 - protein, 16 aa, Homo sapiens
  source 1..16: note = PO2F2_HUMAN.H10MO.D
WMATTTGCATAWTYAW

SEQ ID NO: 568 - protein, 7 aa, Homo sapiens
  source 1..7: note = VDR_HUMAN.H10MO.S
GRGKTCA

SEQ ID NO: 569 - protein, 7 aa, Homo sapiens
  source 1..7: note = PPARA_HUMAN.H10MO.S
RAGGTCA

SEQ ID NO: 570 - protein, 11 aa, Homo sapiens
  source 1..11: note = RUNX3_HUMAN.H10MO.C
NTYTGTGGTTW

SEQ ID NO: 571 - protein, 12 aa, Homo sapiens
  source 1..12: note = ALX4_HUMAN.H10MO.D
TAATYTAATTAR

SEQ ID NO: 572 - protein, 13 aa, Homo sapiens
  source 1..13: note = EPAS1_HUMAN.H10MO.D
GTGHRTRCGTGKG

SEQ ID NO: 573 - protein, 9 aa, Homo sapiens
  source 1..9: note = ATF2_HUMAN.H10MO.B
VTGACGTMA

SEQ ID NO: 574 - protein, 13 aa, Homo sapiens
  source 1..13: note = FOXJ3_HUMAN.H10MO.A
TTGTTTATKKTTW

SEQ ID NO: 575 - protein, 13 aa, Homo sapiens
  source 1..13: note = NKX31_HUMAN.H10MO.C
WWTAAGTATWTWW

SEQ ID NO: 576 - protein, 11 aa, Homo sapiens
  source 1..11: note = MBD2_HUMAN.H10MO.B
SSGKCCGGMGR

SEQ ID NO: 577 - protein, 14 aa, Homo sapiens
  source 1..14: note = HNF4G_HUMAN.H10MO.C
VRGKNCAAAGKYCA

SEQ ID NO: 578 - protein, 11 aa, Homo sapiens
  source 1..11: note = TYY2_HUMAN.H10MO.D
AWAATGGCGGH

SEQ ID NO: 579 - protein, 10 aa, Homo sapiens
  source 1..10: note = BARX2_HUMAN.H10MO.D
TYRWTAATKR

SEQ ID NO: 580 - protein, 15 aa, Homo sapiens
  source 1..15: note = HNF1A_HUMAN.H10MO.A
RGTTAATMWTTAMCM

SEQ ID NO: 581 - protein, 7 aa, Homo sapiens
  source 1..7: note = MAFG_HUMAN.H10MO.S
MATGACT

SEQ ID NO: 582 - protein, 16 aa, Homo sapiens
  source 1..16: note = DLX1_HUMAN.H10MO.D
WWWWWTATGSTAATTA

SEQ ID NO: 583 - protein, 15 aa, Homo sapiens
  source 1..15: note = GCR_HUMAN.H10MO.A
RGRACABKSTGTYCY

SEQ ID NO: 584 - protein, 18 aa, Homo sapiens
  source 1..18: note = NRF1_HUMAN.H10MO.A
SYGCGCMTGCGCRSBGSS

SEQ ID NO: 585 - protein, 17 aa, Homo sapiens
  source 1..17: note = FOXG1_HUMAN.H10MO.D
DTTGTTTAYDTWTDTTT

SEQ ID NO: 586 - protein, 11 aa, Homo sapiens
  source 1..11: note = MIXL1_HUMAN.H10MO.D
GBTAATTARAT

SEQ ID NO: 587 - protein, 16 aa, Homo sapiens
  source 1..16: note = IRF4_HUMAN.H10MO.C
RRRGRGGAASTGARAS

SEQ ID NO: 588 - protein, 16 aa, Homo sapiens
  source 1..16: note = SOX11_HUMAN.H10MO.D
AACAMTGMMATTGTTB

SEQ ID NO: 589 - protein, 13 aa, Homo sapiens
  source 1..13: note = NGN2_HUMAN.H10MO.D
AMCATATGYYWHH

SEQ ID NO: 590 - protein, 13 aa, Homo sapiens
  source 1..13: note = LHX3_HUMAN.H10MO.C
AAAATTAATTARY

SEQ ID NO: 591 - protein, 11 aa, Homo sapiens
  source 1..11: note = PO6F2_HUMAN.H10MO.D
KWTAATKAGST

SEQ ID NO: 592 - protein, 13 aa, Homo sapiens
  source 1..13: note = SOX3_HUMAN.H10MO.D
WSAATGBWATTSW

SEQ ID NO: 593 - protein, 16 aa, Homo sapiens
  source 1..16: note = MEF2B_HUMAN.H10MO.D
TGTTRCTATWWATAGC

SEQ ID NO: 594 - protein, 18 aa, Homo sapiens
  source 1..18: note = AIRE_HUMAN.H10MO.C
HTTGGWWWWAWTGGDTWH

SEQ ID NO: 595 - protein, 11 aa, Homo sapiens
  source 1..11: note = GFI1B_HUMAN.H10MO.C
WGCAGTGATTT

SEQ ID NO: 596 - protein, 13 aa, Homo sapiens
  source 1..13: note = BHE22_HUMAN.H10MO.D
AVCATATGTTTTT

SEQ ID NO: 597 - protein, 9 aa, Homo sapiens
  source 1..9: note = PO2F1_HUMAN.H10MO.B
HATGCAAAT

SEQ ID NO: 598 - protein, 13 aa, Homo sapiens
  source 1..13: note = RORG_HUMAN.H10MO.C
DWAWGTRGGTCAG

SEQ ID NO: 599 - protein, 12 aa, Homo sapiens
  source 1..12: note = TYY1_HUMAN.H10MO.A
CAARATGGCGGC

SEQ ID NO: 600 - protein, 20 aa, Homo sapiens
  source 1..20: note = IRF5_HUMAN.H10MO.D
KAAAGGRAARCMAAAWSWGA

SEQ ID NO: 601 - protein, 22 aa, Homo sapiens
  source 1..22: note = TBX19_HUMAN.H10MO.D
WWTTCRCACVTAGGTGTGAAWW

SEQ ID NO: 602 - protein, 11 aa, Homo sapiens
  source 1..11: note = HMX2_HUMAN.H10MO.D
DTTAAKTGNTT

SEQ ID NO: 603 - protein, 11 aa, Homo sapiens
  source 1..11: note = CREB1_HUMAN.H10MO.A
TKACGTCAYYN

SEQ ID NO: 604 - protein, 11 aa, Homo sapiens
  source 1..11: note = RARB_HUMAN.H10MO.D
SAGGTCAVVVV

SEQ ID NO: 605 - protein, 13 aa, Homo sapiens
  source 1..13: note = FOXO6_HUMAN.H10MO.D
WWSWTGTTTACRT

SEQ ID NO: 606 - protein, 19 aa, Homo sapiens
  source 1..19: note = KAISO_HUMAN.H10MO.A
GYTCTCGCGAGRBYBVGGS

SEQ ID NO: 607 - protein, 11 aa, Homo sapiens
  source 1..11: note = NOTO_HUMAN.H10MO.D
RTTAATTAGNT

SEQ ID NO: 608 - protein, 13 aa, Homo sapiens
  source 1..13: note = DDIT3_HUMAN.H10MO.C
GGGGATTGCAYBB

SEQ ID NO: 609 - protein, 12 aa, Homo sapiens
  source 1..12: note = RAX2_HUMAN.H10MO.D
YTAATTARATWA

SEQ ID NO: 610 - protein, 13 aa, Homo sapiens
  source 1..13: note = TF7L2_HUMAN.H10MO.A
KBYCTTTGAWSTB

SEQ ID NO: 611 - protein, 13 aa, Homo sapiens
  source 1..13: note = ARX_HUMAN.H10MO.D
YTAATYTAATTAR

SEQ ID NO: 612 - protein, 15 aa, Homo sapiens
  source 1..15: note = ZBTB4_HUMAN.H10MO.S
CAATRGCGDTKGYGR

SEQ ID NO: 613 - protein, 9 aa, Homo sapiens
  source 1..9: note = ERR2_HUMAN.H10MO.A
YCAAGGTCA

SEQ ID NO: 614 - protein, 11 aa, Homo sapiens
  source 1..11: note = HXC11_HUMAN.H10MO.D
DTTTWAYGACY

SEQ ID NO: 615 - protein, 11 aa, Homo sapiens
  source 1..11: note = LHX4_HUMAN.H10MO.D
DNRYTAATTRN

SEQ ID NO: 616 - protein, 19 aa, Homo sapiens
  source 1..19: note = FOXL1_HUMAN.H10MO.D
TKTTTGTTTRTTTAYWTTT

SEQ ID NO: 617 - protein, 11 aa, Homo sapiens
  source 1..11: note = HXD11_HUMAN.H10MO.D
DWTTTATKRHN

SEQ ID NO: 618 - protein, 13 aa, Homo sapiens
  source 1..13: note = FOXJ3_HUMAN.H10MO.S
TKKTTWWTGTTTA

SEQ ID NO: 619 - protein, 11 aa, Homo sapiens
  source 1..11: note = RUNX2_HUMAN.H10MO.B
YTGTGGTTDGK

SEQ ID NO: 620 - protein, 16 aa, Homo sapiens
  source 1..16: note = SOX21_HUMAN.H10MO.D
RAACAATGKYAKTGTT

SEQ ID NO: 621 - protein, 8 aa, Homo sapiens
  source 1..8: note = DLX2_HUMAN.H10MO.D
ATAATTRB

SEQ ID NO: 622 - protein, 7 aa, Homo sapiens
  source 1..7: note = ISL1_HUMAN.H10MO.D
SYTAATR

SEQ ID NO: 623 - protein, 10 aa, Homo sapiens
  source 1..10: note = E2F3_HUMAN.H10MO.B
SSCGCSAAAC

SEQ ID NO: 624 - protein, 13 aa, Homo sapiens
  source 1..13: note = PO4F2_HUMAN.H10MO.D
YATTAATGAGYTK

SEQ ID NO: 625 - protein, 15 aa, Homo sapiens
  source 1..15: note = HXA7_HUMAN.H10MO.D
VMTCRATMGATTGGM

SEQ ID NO: 626 - protein, 10 aa, Homo sapiens
  source 1..10: note = IRF7_HUMAN.H10MO.C
GAAASYGAAA

SEQ ID NO: 627 - protein, 11 aa, Homo sapiens
  source 1..11: note = ALX3_HUMAN.H10MO.D
TAATYTAATTA

SEQ ID NO: 628 - protein, 11 aa, Homo sapiens
  source 1..11: note = ZKSC3_HUMAN.H10MO.D
TYCGAGGCTAG

SEQ ID NO: 629 - protein, 13 aa, Homo sapiens
  source 1..13: note = ZN410_HUMAN.H10MO.D
TATTATGGGATGK

SEQ ID NO: 630 - protein, 16 aa, Homo sapiens
  source 1..16: note = MGAP_HUMAN.H10MO.D
AGGTGTGAAWTTTCAC

SEQ ID NO: 631 - protein, 14 aa, Homo sapiens
  source 1..14: note = OLIG1_HUMAN.H10MO.D
AMCATATGTTTTTY

SEQ ID NO: 632 - protein, 14 aa, Homo sapiens
  source 1..14: note = CUX1_HUMAN.H10MO.C
RBRSBDATCGATSK

SEQ ID NO: 633 - protein, 17 aa, Homo sapiens
  source 1..17: note = CPEB1_HUMAN.H10MO.D
TTTTTATTYTTTTTTTT

SEQ ID NO: 634 - protein, 15 aa, Homo sapiens
  source 1..15: note = HSF4_HUMAN.H10MO.D
TRGAABVTTCTAGAA

SEQ ID NO: 635 - protein, 7 aa, Homo sapiens
  source 1..7: note = HMGA1_HUMAN.H10MO.D
NWATTTT

SEQ ID NO: 636 - protein, 15 aa, Homo sapiens
  source 1..15: note = CDC5L_HUMAN.H10MO.D
GBGWKTTAAYRTAWY

SEQ ID NO: 637 - protein, 13 aa, Homo sapiens
  source 1..13: note = MEF2A_HUMAN.H10MO.A
DKCTATTTTTRGM

SEQ ID NO: 638 - protein, 11 aa, Homo sapiens
  source 1..11: note = SUH_HUMAN.H10MO.C
SYGTGRGAAMN

SEQ ID NO: 639 - protein, 25 aa, Homo sapiens
  source 1..25: note = BRAC_HUMAN.H10MO.D
RSAWWGTGACACCTAGGTGTGAAAT

SEQ ID NO: 640 - protein, 16 aa, Homo sapiens
  source 1..16: note = SOX7_HUMAN.H10MO.D
AACAMTGMAATTGTTY

SEQ ID NO: 641 - protein, 9 aa, Homo sapiens
  source 1..9: note = FOXO4_HUMAN.H10MO.C
TTGTTTAYK

SEQ ID NO: 642 - protein, 21 aa, Homo sapiens
  source 1..21: note = CUX2_HUMAN.H10MO.D
DATCRATWHDDTTATCGATYA

SEQ ID NO: 643 - protein, 11 aa, Homo sapiens
  source 1..11: note = HXD12_HUMAN.H10MO.D
DDTTTTAHKRN

SEQ ID NO: 644 - protein, 14 aa, Homo sapiens
  source 1..14: note = ETV3_HUMAN.H10MO.D
KWMRTTTGCACTTC

SEQ ID NO: 645 - protein, 11 aa, Homo sapiens
  source 1..11: note = ZFHX3_HUMAN.H10MO.D
ATTAWTAATTA

SEQ ID NO: 646 - protein, 17 aa, Homo sapiens
  source 1..17: note = CENPB_HUMAN.H10MO.D
TTCGTWSBAHGCGGGWW

SEQ ID NO: 647 - protein, 12 aa, Homo sapiens
  source 1..12: note = BHE23_HUMAN.H10MO.D
RRSRTRTGKTDB

SEQ ID NO: 648 - protein, 15 aa, Homo sapiens
  source 1..15: note = IRF8_HUMAN.H10MO.D
GRGRAAVTGAAASYR

SEQ ID NO: 649 - protein, 7 aa, Homo sapiens
  source 1..7: note = MSX2_HUMAN.H10MO.D
TAATTDK

SEQ ID NO: 650 - protein, 11 aa, Homo sapiens
  source 1..11: note = SOX9_HUMAN.H10MO.B
YCCATTGTTYT

SEQ ID NO: 651 - protein, 11 aa, Homo sapiens
  source 1..11: note = BHA15_HUMAN.H10MO.D
BDCCATATGKT

SEQ ID NO: 652 - protein, 19 aa, Homo sapiens
  source 1..19: note = HXC10_HUMAN.H10MO.D
WWWWWWWNNWTTTWATKRC

SEQ ID NO: 653 - protein, 17 aa, Homo sapiens
  source 1..17: note = MCR_HUMAN.H10MO.D
HAGDMCWGGTTGWDGTR

SEQ ID NO: 654 - protein, 15 aa, Homo sapiens
  source 1..15: note = HOMEZ_HUMAN.H10MO.D
WWWAATCGTTTTYTW

(19) United States
(12) Patent Application Publication
(10) Pub. No.: US 2024/0141050 A1
(43) Pub. Date: May 2, 2024

(54) TREATMENTS FOR PRURIGO NODULARIS

(71) Applicants: GALDERMA HOLDING S.A., Zug (CH); Chugai Seiyaku Kabushiki Kaisha, Tokyo (JP)

(72) Inventors: Valerie JULIA, Biot (FR); Jayendra Kumar KRISHNASWAMY, Pully (CH); Christophe PIKETTY, Montargis (FR); Francois ROUSSEAU, Publier (FR)

(21) Appl. No.: 18/238,398

(22) Filed: Aug. 25, 2023

Related U.S. Application Data
(60) Provisional application No. 63/403,483, filed on Sep. 2, 2022.
(60) Provisional application No. 63/534,558, filed on Aug. 24, 2023.

(51) Int. Cl. C07K 16/28; A61K 39/00; A61P 17/04; A61P 29/00; C12Q 1/6883

(52) U.S. Cl. CPC C07K 16/2866; A61P 17/04; A61P 29/00; C12Q 1/6883; A61K 2039/505; C07K 2317/565; C12Q 2600/106; C12Q 2600/158

(57) ABSTRACT

Described herein are treatments and preventions for prurigo nodularis (PN), antibodies and pharmaceutical compositions for use in the treatment or prevention of PN, and uses of an anti-IL-31RA antibody (e.g., nemolizumab) in the manufacture of a medicament for the treatment or prevention of PN. Also described herein are biomarkers of PN and methods of altering or improving these biomarkers via treatments with an antibody that binds to IL-31RA (e.g., nemolizumab).

### RELATED APPLICATIONS

This application claims priority under 35 U.S.C. § 119(e) to (i) U.S. Provisional Application No. 63/403,483 filed Sep. 2, 2022, and (ii) U.S. Provisional Application No. 63/534,558 filed Aug. 24, 2023, the entire contents of both which are incorporated herein by reference.

### SEQUENCE LISTING

The instant application contains a Sequence Listing which has been submitted electronically in XMLformat and is hereby incorporated by reference in its entirety. Said XML copy, created on Jan. 18, 2024, is named 105153-1354_SL.xml and is 669,000 bytes in size.

### FIELD

Described herein are treatments and preventions prurigo nodularis (PN), antibodies and pharmaceutical compositions for use in the treatment or prevention of PN, and uses of an anti-IL-31RA antibody (e.g., nemolizumab) in the manufacture of a medicament for the treatment or prevention of PN. Also described herein are biomarkers of PN and methods of altering or improving these biomarkers via treatments with an antibody that binds to IL-31RA (e.g., nemolizumab).

### BACKGROUND

The following discussion is provided to aid the reader in understanding the disclosure and is not admitted to describe or constitute prior art thereto.

Chronic prurigo (CP) is a skin disease due to neuronal sensitization to itch and development of an itch-scratch cycle. Prurigo nodularis (PN), a subtype of CP, is a skin disease that causes hard, itchy lumps (nodules) to form on the skin. The itching (pruritus) can be intense, causing people to scratch themselves to the point of bleeding or pain. Scratching can cause more skin lesions to appear. The itching is worsened by heat, sweating, or irritation from clothing. In some cases, people with PN have a history of other diseases including eczema (atopic dermatitis), diabetes, lymphoma, HIV infection, severe anemia, or kidney disease.

The exact cause of PN is unknown, and diagnosis of the disease is based on observing signs such as extremely itchy skin with the formation of nodules. In some cases, a skin biopsy is used to confirm the diagnosis. Currently treatment may include corticosteroid creams, oral medications, cryotherapy, or photochemotherapy.

There remains a need for treatments for PN and identifying whether patients are likely to respond or are responding to such treatments.

### SUMMARY

Described herein are treatments and preventions for prurigo nodularis (PN) that achieve particular therapeutic results, such as a decrease in TNF signaling. In general, the treatments and preventions comprise administering to a subject with PN an anti-IL-31RA antibody (e.g., nemolizumab). Also described herein are biomarkers of PN and methods of using the disclosed biomarkers to determine whether a subject is responsive to treatment.

In a first aspect, the present disclosure provides methods of treating or preventing prurigo nodularis (PN) in a subject, comprising administering to a subject with PN an anti-IL-31RA antibody, wherein the subject shows activation of tumor necrosis factor (TNF) signaling in a lesional skin cell compared to a reference level of activation of TNF.

In a second aspect, the present disclosure provides methods of normalizing expression of a tumor necrosis factor (TNF) gene in a subject with PN, comprising administering to a subject with PN an anti-IL-31RA antibody, wherein the subject shows activation of tumor necrosis factor (TNF) in a lesional skin cell compared to a reference level of activation of TNF and wherein administration of the anti-IL-31RA antibody normalizes the activation of TNF signaling. In some embodiments, normalization is determined about 4 weeks, about 8 weeks, or about 12 weeks after administration of the anti-IL-31RA antibody.

In some embodiments, differential expression was determined by RT-qPCR, RT-PCR, RNA-seq, Northern blotting, Serial Analysis of Gene Expression (SAGE), or DNA or RNA microarrays; or wherein differential expression was determined at protein level by Western blotting, ELISA, surface plasmon resonance, or mass spectrometry.

In some embodiments, the activation of TNF signaling in the lesional skin cell is higher as compared to the reference level. In some embodiments, the lesional skin cell is a fibroblast

In some embodiments, the reference level is the level of activation is a level of activation of the TNF signaling in a skin cell of a person that does not have PN. In some embodiments, the skin cell of the person that does not have PN is a fibroblast.

In some embodiments, the reference level is the level of activation is a level of activation of the TNF signaling in a non-lesional skin cell of the subject.

In a third aspect, the present disclosure provides methods of decreasing inflammation in the skin of a subject with prurigo nodularis (PN), comprising administering to a subject with PN an anti-IL-31RA antibody, thereby decreasing inflammation involving tumor necrosis factor (TNF) signaling in the skin.

In some embodiments, TNF signaling in skin of the subject is overexpressed relative to a reference level of activation of TNF signaling, optionally, wherein the TNF gene is overexpressed by a fibroblast.

In some embodiments, the reference level is the level of activation is a level of activation of the TNF signaling in a skin cell of a person that does not have PN. In some embodiments, the skin cell of the person that does not have PN is a fibroblast.

In some embodiments, the reference level is the level of activation is a level of activation of the TNF signaling in a non-lesional skin cell of the subject.

In some embodiments, the inflammation further involves IL-1 pathway signaling, IL-6 pathway signaling, TGFβ pathway signaling, or any combination thereof.

In a fourth aspect, the present disclosure provides methods of treating or preventing prurigo nodularis (PN) in a subject, comprising administering to a subject with PN an anti-IL-31RA antibody, wherein treatment with the anti-IL-31RA antibody results in a decrease in tumor necrosis factor (TNF) pathway activation. In some embodiments, the decrease in TNF pathway activation occurs in lesional skin of the subject. In some embodiments, the decrease in TNF pathway activation occurs in a fibroblast of the subject.

In some embodiments, the treatment further results is:
(a) a decrease in migration of leukocytes or cell movement of leukocytes;
(b) an inhibition of a STAT3 pathway;
(c) an inhibition of a STAT5b pathway;
(d) a downregulation of IL-1 or an IL-1 pathway;
(e) a downregulation of IL-6 or an IL-6 pathway;
(f) a downregulation of VEGF or a VEGF pathway;
(g) a decrease in TGFB1 pathway activation, or
(h) a combination thereof.

In some embodiments, (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is determined relative to (i) a control sample obtained from an individual or individuals without PN or (ii) a biological sample obtained from the subject prior to administration of the anti-IL-31RA antibody.

In some embodiments, (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is assessed after about 4 weeks, about 8 weeks, or about 12 weeks after the administration of the anti-IL-31RA antibody.

In some embodiments, (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is determined by mass spectrometry performed on one or more biological sample(s) obtained from the subject.

In some embodiments, the one or more biological sample(s) is a plasma sample or a skin sample.

In some embodiments, the subject exhibits at least two of, at least three of, at least four of, at least five of, at least six, or all seven of (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; and (g) the decrease in TGFB1 pathway activation.

In a fifth aspect, the present disclosure provides methods of deactivating, decreasing activation, or decreasing a number of COL11A1+ fibroblasts in a subject with prurigo nodularis (PN), comprising administering to the subject an anti-IL-31RA antibody, wherein administering the anti-IL-31RA antibody results in deactivation, a decrease in activation, or a decrease in number of COL11A1+ fibroblasts in the subject's skin. In some embodiments, the COL11A1+ fibroblasts are found in the papillary dermis.

In a sixth aspect, the present disclosure provides methods of decreasing TGFβ expression in at least one cell type in a subject with PN, comprising administering to the subject an anti-IL-31RA antibody, wherein administering the anti-IL-31RA antibody results in a decrease in TGFβ expression in at least one cell type in the subject's skin. In some embodiments, the at least one cell type comprises fibroblasts, endothelial cells, pericytes, nerve cells, or any combination thereof. In some embodiments, the decrease in TGFβ expression comprises a decrease in the expression of TGFB1, TGFB2, TGFB3, or any combination thereof.

In a seventh aspect, the present disclosure provides methods of decreasing expression at least one inflammatory gene expressed by a keratinocyte in a subject with PN, comprising administering to the subject an anti-IL-31RA antibody, wherein administering the anti-IL-31RA antibody results in a decrease in at least one inflammatory gene expressed by a keratinocyte in the subject's skin. In some embodiments, the at least one inflammatory gene is selected from KRT6, KRT16, KRT17, S100A8, S100A9, and any combination thereof. In some embodiments, the keratinocyte expresses Th2 cytokines. In some embodiments, administering the anti-IL-31RA antibody results in a decrease in reactive oxygen species and/or cellular stress to which the keratinocyte is exposed.

In an eighth aspect, the present disclosure provides methods of decreasing infiltration of at least one type of immune cell in a skin lesion of a subject with PN, comprising administering to the subject an anti-IL-31RA antibody, wherein administering the anti-IL-31RA antibody results in a decrease in infiltration of at least one type of immune cell in at least one lesion in the subject's skin. In some embodiments, the at least one type of immune cell comprises a macrophage. In some embodiments, the macrophage is a lipid-associated macrophage characterized by expression of APOE and TREM2. In some embodiments, the at least one type of immune cell comprises T cells, NK cells, CD8⁺ cells, Tregs and any combination thereof. In some embodiments, includes administering the anti-IL-31RA antibody results in a decrease in expression of ICAM1, E-selectin (SELE), IL6 CCL2, CCL3, CCL4, CCL13, CCL18, CXCL2, CXCL12, and any combination thereof in at least one cell type in the lesion. In some embodiments, the at least on cell type in the lesion comprises myeloid cells, pericytes, endothelial cells, and any combination thereof.

In some embodiments of any of the foregoing aspects, the anti-IL-31RA antibody is administered subcutaneously.

In some embodiments of any of the foregoing aspects, the anti-IL-31RA antibody is administered once per week, once every two weeks, once every three weeks, once every four weeks, once every five weeks, once every six weeks, once every seven weeks, or once every eight weeks.

In some embodiments of any of the foregoing aspects, the anti-IL-31RA antibody is administered at a dose of about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg. Alternatively, in some embodiments, the anti-IL-31RA antibody is administered at a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg.

In some embodiments of any of the foregoing aspects, the anti-IL-31RA antibody is administered according to a flat dosing regimen. Alternatively, in some embodiments, the anti-IL-31RA antibody is administered according to a loading dose regimen.

In some embodiments of any of the foregoing aspects, the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14. In some embodiments, the anti-IL-31RA antibody is nemolizumab or a fragment or variant thereof. In some embodiments, the anti-IL-31RA antibody is nemolizumab.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIGS. 1A-1E shows that prurigo nodularis (PN) is characterized by immune activation and abnormal keratinocyte differentiation. Number of differentially expressed genes (DEGs) in PN lesional vs. non-lesional skin (n=62, FC>=2 or FC<=−2, FDR<=0.1) (FIG. 1A). Enriched GO categories in PN lesional skin (FIG. 1i). Literature-based gene network obtained from the top 1,000 DEGs in PN skin, generated using Genomatix Pathway System (GePS, Genomatix.de). The picture displays the top 50 best connected genes co-cited in PubMed abstracts in the same sentence linked to a function word (most relevant genes/interactions). Orange represent the genes that are upregulated and green represent the genes that are downregulated in PN lesional vs. non-lesional skin. Critical nodes included the proliferation markers Ki67 (MKI67), IL-1 family members IL36G and ILIA, and CXCL8 and CDKN1A (FIG. 1C). Number of genes and within cluster correlation in the modules identified from the weighted gene co-expression gene networks (WCGNA) analysis of non-lesional and lesional PN skin (FIG. 1D). Functions enriched in key co-expression modules from PN skin (module #5, #6 and #8) (FIG. 1E).

FIGS. 2A-2C shows enriched transcriptomic cellular signatures and overlap with psoriasis and AD. Cell type inference analysis on non-lesional (NL) and lesional (L) PN skin samples using xCell. Enriched cellular signatures are shown as red but underrepresented cellular signatures are shown in blue. The bar on the left side shows statistical difference in the enrichment between lesional and non-lesional PN skin with the colors representing different p-value thresholds (FIG. 2A). Comparison of PN associated DEGs against DEGs in Psoriasis (Pso) and atopic dermatitis (AD) for increased and decreased DEGs (FIG. 2B). Correlation analysis between the effect sizes in PN lesion versus those in psoriasis (Pso) and atopic dermatitis (AD). Spearman's rank-order correlation was included. The genes significant in the x-axis, y-axis, and both axes are colored in red/blue/purple respectively (FIG. 2C).

FIGS. 3A-3B shows transcriptomic changes associated with the anti-IL31R inhibitor nemolizumab. Principal component analyses (PCA) of the transcriptomic data from PN biopsies prior to and after 12-weeks of prospective placebo controlled, double blinded clinical trial with the anti-IL-31R inhibitor nemolizumab. Different colors represent different treatment groups, with lesional samples shown as triangles, and non-lesional skin shown as circles (FIG. 3A). Heatmap showing 2-way clustering (using genes differentially expressed between non-lesional vs lesional skin at baseline) for all samples (FIG. 3B).

FIGS. 4A-4C shows the effect of nemolizumab on PN associated transcriptomic changes. 3-way Venn diagram of increased (FIG. 4A) and decreased (FIG. 4B) DEGs in PN skin and overlap with DEGs (compared to baseline) in the nemolizumab and placebo cohorts. Correlation analyses between different groups (PN baseline vs. placebo and nemolizumab DEGs) (Spearman's rank-order correlation) (FIG. 4C).

FIGS. 5A-5D shows nemolizumab treatment leads to normalized epidermal differentiation and decreased IL-31/Th2 responses in PN skin. Nemolizumab treatment led to decrease in IL-31 and IL-13 responses in PN skin compared to placebo, along with decreased expression of IL-17A response genes (FIG. 5A). Nemolizumab treatment was accompanied by decreases in transcriptomic signatures for Th1, Th17 and Th2 cells (FIG. 5B). Cross comparing transcriptomic responses in PN skin against cellular signatures obtained from single-cell data of healthy epidermis demonstrated that nemolizumab treatment led to normalization of epidermal gene expression related to the differentiated layer (KRT10+) of the epidermis, corresponding to normalization of epidermal differentiation. The different nomenclatures correspond to the different layers of the epidermis with “basal” corresponding to KRT5⁺ basal cells, KRT10⁺ “differentiated” corresponding to the spinous layer, and “keratinized” corresponding to the granular layers (FLG⁺) (FIG. 5C). Transcription Factor Binding Site (TFBS) of PN associated DEGs that greater normalization amongst nemolizumab down-regulated genes compared to placebo (FIG. 5D).

FIGS. 6A-6B shows nemolizumab driven decrease in pruritus scoring was accompanied by tighter clustering of PN samples on PCA analyses after 12 weeks of treatment. The peak pruritus numeric rating scale (PP-NRS) was superimposed on transcriptomic data from PN lesional skin and assessed using PCA analyses. Nemolizumab group is shown as large dots, whereas placebo group is shown as small dots. Baseline (top) and week 12 of treatment (bottom) are shown. (FIG. 6A). Nemolizumab treatment led to tighter clustering of PN samples on PCA analyses compared to biopsy samples from the placebo treated cohort (FIG. 6B).

FIG. 7 shows cytokine signatures in PN in placebo vs. nemolizumab treated skin. Literature-based network generated using Genomatix Pathway System (GePS, genomatix.de), using the function-word co-citation filter and showing key cytokines as main nodes in PN skin.

FIG. 8 shows expression of selected TFs in PN skin pre- and post-nemolizumab treatment.

FIG. 9 shows the distance between PCA components in PN, pre- and post-nemolizumab treatment.

FIG. 10 shows the clinical scores of the patients selected for mass spectrometry analysis. Left panel: PNR scores at baseline. Right panel: NRS change at week 12.

FIG. 11 shows enriched canonical pathways from mass spectrometry. Enriched canonical pathways were sorted according to: right) z-scores only, left) z-scores and p-value (p<0.05).

FIG. 12 shows upstream regulator analysis from mass spectrometry. Above: Upstream regulators were sorted according to: right) z-scores only, left) z-scores and p-value (p<0.05).

FIG. 13 shows enriched biological functions as identified by mass spectrometry. Enriched biological function ontologies were sorted according to z-scores and p-values (p<0.05).

FIG. 14 shows a hierarchical clustering heatmap based on scRNAseq of the samples obtained from healthy skin (H), lesional PN (LPN) and non-lesional PN (NPN) skin samples. Y-axis represents the single cells, while X-axis represents genes expressed by them. The right-hand side of the heatmap indicates the cell source and cell-type annotations.

FIG. 15 shows a UMAP representation of the Fibroblast cluster based on scRNAseq. Clusters annotated with disease type, Fibroblast sub-type, cell differentiation trajectory and pseudo-time.

FIG. 16 shows a graphical summary of lesional PN fibroblast DEGs using Ingenuity pathway analysis. Orange color denotes the pathways, regulators or processes activated in the lesional PN skin compared to healthy skin, while blue denotes de-activation.

FIG. 17 shows cell-cell communication networks in PN lesional skin. Data are derived from scRNAseq data. The higher the number of interactions/interaction strength, the thicker the line connecting two cell-types.

FIG. 18 shows a graphical summary of the results obtained from Ingenuity pathway analysis of bulk-RNAseq data from phase II study on nemolizumab. Orange color denotes the pathways, regulators or processes activated in the lesional PN skin of nemolizumab-treated patients compared to healthy skin, while blue denotes de-activation.

FIGS. 19A-19E show cell types observed in PN skin and their spatial locations. FIG. 19A shows a UMAP plot showing 72,782 cells colored by cell types. FIG. 19B shows a UMAP plot showing the cells colored by skin conditions (H: healthy control; NPN: nonlesional samples from patients with PN; LPN: lesional samples from patients with PN). FIG. 19C shows a bar plot showing the abundance composition across the skin conditions for each cell type in scRNA-seq. FIG. 19D shows a dot plot showing representative marker genes for each cell type. The color scale represents the scaled expression of each gene. The size of the dot represents the percentage of cells expressing each gene of interest. FIG. 19E shows a spatial plot showing the prediction score for each cell type. The coordinates of the spot correspond to the location in the tissue.

FIGS. 20A-20C show spatial locations of the major cell types detected in PN skin. FIG. 20A shows hematoxylin & eosin (H&E) staining of the PN skin biopsy used for spatial sequencing. FIG. 20B shows a scatter pie plot showing the cell type composition for each spot in the spatial-seq sample. Each spot is represented as a pie chart showing the relative proportion of the cell types. FIG. 20C shows a spatial plot showing the extracellular matrix score in the spatial-seq sample.

FIGS. 21A-21J show identification of fibroblast subtypes. FIG. 21A shows a UMAP showing 15,084 fibroblasts colored by subtypes. FIG. 21B shows a UMAP plot showing the fibroblasts colored by skin conditions. FIG. 21C shows a bar plot showing the abundance composition across the skin conditions for each fibroblast subtype. FIG. 21D shows a dot plot showing the top marker genes for each fibroblast subtype. The color scale represents the scaled expression of each gene. The size of the dot represents the percentage of cells expressing the gene of interest. FIG. 21E shows violin plots showing the extracellular matrix module score in the fibroblast subtypes split by the skin conditions. FIG. 21F shows a dot plot showing the upstream regulators for the DEGs identified in COL11A+FB by comparing LPN to healthy cells. The color scale represents the −log 10(p-value) from the enrichment analysis. The size of the dot represents the number of differentially expressed genes downstream of the upstream regulator. FIG. 21G shows a bar plot showing the top10 pathways enriched using the up-regulated DEGs identified in COL11A+FB by comparing LPN to healthy cells. FIG. 21H shows a dot plot showing the expression of all the collagen genes across the fibroblast subtypes. The color scale represents the scaled expression of each gene. The size of the dot represents the percentage of cells expressing the gene of interest. FIG. 21I shows immunohistochemistry staining for Trichrome, Pro-collagen I and COL11A1 in PN and healthy tissues. FIG. 21J shows violin plots showing the extracellular matrix module score in the fibroblast subtypes split by the healthy, PN, and AD skin conditions.

FIG. 22 shows identification of fibroblast subtypes. This figure provides immunohistochemistry staining for SFRP4, SFRP2, and RAMP1 in PN and healthy tissues.

FIGS. 23A-23H show identification of endothelial subtypes. FIG. 23A shows a UMAP plot showing 3,840 endothelial cells colored by sub-clusters. FIG. 23B shows a dot plot showing the top marker genes for each endothelial sub-cluster. The color scale represents the scaled expression of each gene. The size of the dot represents the percentage of cells expressing the gene of interest. FIG. 23C shows a UMAP plot showing the endothelial cells colored by skin conditions. FIG. 23D shows a bar plot showing the abundance composition across the skin conditions for each endothelial sub-cluster. FIG. 23E shows a dot plot showing the upstream regulators for the cluster marker genes for endothelial sub-cluster 2. The color scale represents the −log 10(p-value) from the enrichment analysis. The size of the dot represents the number of differentially expressed genes downstream of the upstream regulator. FIG. 23F shows a bar plot showing the top10 pathways enriched using the cluster marker genes for endothelial sub-cluster 2. FIG. 23G shows a dot plot showing the upstream regulators for the cluster marker genes for endothelial sub-cluster 5. The color scale represents the −log 10(p-value) from the enrichment analysis. The size of the dot represents the number of differentially expressed genes downstream of the upstream regulator. FIG. 23H shows a bar plot showing the top10 pathways enriched using the cluster marker genes for endothelial sub-cluster 5.

FIGS. 24A-24J show identification of pericyte subtypes. FIG. 24A shows a UMAP plot showing 3,052 pericytes colored by sub-clusters. FIG. 24B shows a UMAP plot showing the pericytes colored by skin conditions. FIG. 24C shows a bar plot showing the abundance composition across the skin conditions for each pericyte sub-cluster. FIG. 24D shows a dot plot showing the top marker genes for each pericyte sub-cluster. The color scale represents the scaled expression of each gene. The size of the dot represents the percentage of cells expressing the gene of interest. FIG. 24E shows violin plots showing the extracellular matrix module score in the pericyte sub-clusters split by the skin conditions. FIG. 24F shows a dot plot showing the expression of all the collagen genes across the pericyte sub-clusters. The color scale represents the scaled expression of each gene. The size of the dot represents the percentage of cells expressing the gene of interest. FIG. 24G shows a dot plot showing the upstream regulators for the cluster marker genes for pericyte sub-cluster 3. The color scale represents the −log 10(p value) from the enrichment analysis. The size of the dot represents the number of differentially expressed genes in the downstream of the upstream regulator. FIG. 24H shows a dot plot showing the upstream regulators for the cluster marker genes for pericyte sub-cluster 7. The color scale represents the −log 10(p value) from the enrichment analysis. The size of the dot represents the number of differentially expressed genes in the downstream of the upstream regulator. FIG. 24I shows a bar plot showing the top10 pathways enriched using the cluster marker genes for pericyte sub-cluster 3. FIG. 24J shows a bar plot showing the top10 pathways enriched using the cluster marker genes for pericyte sub-cluster 7.

FIGS. 25A-25F show identification of keratinocyte subtypes. FIG. 25A shows a UMAP showing 40,277 keratinocytes colored by subtypes. FIG. 25B shows a UMAP plot showing the keratinocytes colored by skin conditions. FIG. 25C shows a bar plot showing the abundance composition across the skin conditions for each keratinocyte subtype. FIG. 25D shows a dot plot showing the top marker genes for each keratinocyte subtype. The color scale represents the scaled expression of each gene. The size of the dot represents the percentage of cells expressing the gene of interest. FIG. 25E shows a bar plot showing the top10 pathways enriched using the cluster marker genes for the inflammatory keratinocytes. FIG. 25F shows a dot plot showing the upstream regulators for the cluster marker genes for the inflammatory keratinocytes. The color scale represents the −log 10(p-value) from the enrichment analysis. The size of the dot represents the number of differentially expressed genes downstream of the upstream regulator.

FIGS. 26A-26J show identification of myeloid and T cell subtypes. FIG. 26A shows a UMAP plot showing 2,130 myeloid cells colored by subtypes. FIG. 26B shows a UMAP plot showing the myeloid cells colored by skin conditions. FIG. 26C shows a dot plot showing the top marker genes for each myeloid subtype. The color scale represents the scaled expression of each gene. The size of the dot represents the percentage of cells expressing the gene of interest. FIG. 26D shows a bar plot showing the abundance composition across the skin conditions for each myeloid subtype. FIG. 26E shows immunohistochemistry staining for TREM2 and CD138 in PN skin tissues. FIG. 26F shows a UMAP plot showing 5,817 T cells colored by subtypes. FIG. 26G shows a UMAP plot showing the T cells colored by skin conditions. FIG. 26H shows a dot plot showing the top marker genes for each T cell subtype. The color scale represents the scaled expression of each gene. The size of the dot represents the percentage of cells expressing the gene of interest. FIG. 26I shows a bar plot showing the abundance composition across the skin conditions for each T cell subtype. FIG. 26J shows immunohistochemistry staining for CD8 and CD4 in PN skin tissues.

FIGS. 27A-27F show cell-cell interactions revealed by ligand-receptor analysis. FIG. 27A shows a heatmap showing the number of ligand-receptor pairs in the healthy samples. Row, cell type expressing the ligand; column, cell type expressing the receptor. Color scale, number of ligand-receptor pairs. FIG. 27B shows a heatmap showing the number of ligand-receptor pairs in the NPN samples. Row, cell type expressing the ligand; column, cell type expressing the receptor. Color scale, number of ligand-receptor pairs. FIG. 27C shows a heatmap showing the number of ligand-receptor pairs in the LPN samples. Row, cell type expressing the ligand; column, cell type expressing the receptor. Color scale, number of ligand-receptor pairs. FIG. 27D shows dot plots showing the expression of specific ligands (left) and receptors (right) that have a higher interaction score in LPN compared to healthy samples. Color scale indicates the level of expression in the cells, whereas dot size reflects the percentage of cells expressing the gene. FIG. 27E shows a cell-cell interactions based on interaction weights/strengths using CellChat. FIG. 27F shows a heatmap of the TGFb signaling pathway network in healthy, NPN, and LPN skin outlining source (sender) and target cell (receiver, mediator, influencer).

FIGS. 28A-28F show a comparison of atopic dermatitis (AD) and prurigo nodularis (PN) keratinocyte subtypes. FIG. 28A shows a UMAP plot showing 68,451 keratinocytes colored by skin state. FIG. 28B shows a UMAP plot showing keratinocytes colored by clustering. FIG. 28C shows a UMAP plot showing keratinocytes colored by keratinocyte subtype. FIG. 28D shows a dot plot showing the top marker genes for each keratinocyte cluster. The color scale represents the scaled expression of each gene. The size of the dot represents the percentage of cells expressing the gene of interest. FIG. 28E shows a bar plot showing the number of keratinocytes in each cluster for each skin state. FIG. 28F shows enriched GO BP Processes in AD vs. PN skin for each keratinocyte subtype.

FIGS. 29A-29H show a comparison of atopic dermatitis (AD) and prurigo nodularis (PN) T cell and myeloid subtypes. FIG. 29A shows a UMAP plot showing 10,389 T cells colored by cluster. FIG. 29B shows a UMAP plot showing colored by T cell subtype. FIG. 29C shows a UMAP plot showing T cells colored by disease state. FIG. 29D shows bar plots showing the abundance and numbers for each T cell subtype across each disease state. FIG. 29E shows a UMAP plot showing 5,752 myeloid cells colored by cluster. FIG. 29F shows a UMAP plot showing myeloid cells colored by myeloid subtype. FIG. 29G shows a UMAP plot showing myeloid colored by disease state. FIG. 29H shows bar plots showing the abundance and numbers for each myeloid subtype across each disease state.

FIGS. 30A-30B shows identification of myeloid and T cell subtypes. FIG. 30A shows a UMAP plot expression of specific immune cells in lesional AD (LAD) and lesional PN skin (LPN). FIG. 30B shows violin plots showing expression in each T cell subset for specific immune genes.

FIGS. 31A-31H show nemolizumab effects in keratinocyte and fibroblast subtypes. FIG. 31A shows a UMAP plot showing the expression of IL31RA mainly in keratinocytes and fibroblasts. FIG. 31B shows a UMAP plot showing the expression of OSMR mainly in keratinocytes, fibroblasts, endothelial cells, and pericytes. FIG. 31C shows a UMAP showing overlay of DEGs increased with nemolizumab treatment (nemolizumab positive). FIG. 31D shows a UMAP showing overlay of DEGs decreased with nemolizumab treatment (nemolizumab negative). FIG. 31E shows a violin plot showing the nemolizumab upregulated gene module score in the fibroblast subtypes split by the skin conditions. FIG. 31F shows a violin plot showing the nemolizumab downregulated gene module score in the fibroblast subtypes split by the skin conditions. FIG. 31G shows a violin plot showing the nemolizumab upregulated gene module score in the keratinocyte subtypes split by the skin conditions. FIG. 31H shows a violin plot showing the nemolizumab downregulated gene module score in the keratinocyte subtypes split by the skin conditions.

FIGS. 32A-32B shows nemolizumab effects across the major cell types in scRNA-seq. FIG. 32A shows a violin plot showing the nemolizumab upregulated gene module score in all the cell types split by the skin conditions. FIG. 32B A shows a violin plot showing the nemolizumab down-regulated module score in all the cell types split by the skin conditions.

### DETAILED DESCRIPTION

Described herein are treatments and preventions of prurigo nodularis (PN) using an anti-IL-31RA antibody (e.g., nemolizumab), as well as biomarkers and gene signatures associated with PN that were never previously known. The disclosed biomarkers, which include differentially expressed genes (DEGs), PN-specific gene ontologies (GOs), and other inflammatory and hyperproliferative markers, can be used to identify a subject with PN, determine whether a subject is likely to respond to treatment (e.g., with an anti-IL-31RA antibody), and track a subject's responsiveness to treatment. The disclosed treatments and preventions achieve therapeutic endpoints (e.g., normalizing DEGs, normalizing epidermal hyperproliferation, normalizing epidermal differentiation, and/or decreasing inflammatory responses in the skin) that were not previously known or obtainable with conventional treatments for PN.

Further, histological analysis of PN nodules described herein reveals epidermal dysregulation (hyperorthokeratosis and hyperplasia), dermal fibrosis and inflammatory cell infiltrate. Thus, the present disclosure also provides the role of fibroblasts in PN pathogenesis and related clinical changes, outcomes, and endpoints achieved by nemolizumab.

### I. DEFINITIONS

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the phrase “therapeutically effective amount” with reference to an anti-IL31R antibody (e.g. nemolizumab) means that dose of the antibody that provides the specific pharmacological effect for which the drug is administered in a subject in need of such treatment. A therapeutically effective amount may be effective to reduce, ameliorate, or eliminate itching, scratching, and/or lesion or nodule formation and/or improve quality of life in a subject with PN. It is emphasized that a therapeutically effective amount of an anti-IL31R antibody (e.g. nemolizumab) will not always be effective in treating PN in every individual subject, even though such dose is deemed to be a therapeutically effective amount by those of skill in the art. Those skilled in the art can adjust what is deemed to be a therapeutically effective amount in accordance with standard practices as needed to treat a specific subject. A therapeutically effective amount may vary based on, for example, the age and weight of the subject, and/or the subject's overall health, and/or the severity of the subject's PN.

The terms “treat,” “treatment” or “treating” as used herein with reference to PN refer to reducing, ameliorating, or eliminating itching, scratching, and/or lesion or nodule formation and/or improving quality of life in a subject with PN.

The terms “prevent,” “preventing” or “prevention” as used herein with reference to a PN refer to precluding or reducing the risk of developing lesions or nodules or preventing the development of the disclosed biomarker signatures that are associated with PN. Prevention may also refer to the prevention of a PN flare or recurrence once an initial flare has been treated or cured.

The terms “individual,” “subject,” and “patient” are used interchangeably herein, and refer to any individual mammalian subject, e.g., bovine, canine, feline, equine, or human. In specific embodiments, the subject, individual, or patient is a human.

### II. PRURIGO NODULARIS (PN) AND BIOMARKERS

Prurigo nodularis (or “PN”) is a skin disease that causes hard, itchy lumps (nodules) to form on the skin. The itching (pruritus) can be intense, causing people to scratch themselves to the point of bleeding or pain. Scratching can cause more skin lesions to appear. The itching is worsened by heat, sweating, or irritation from clothing. In some cases, people with PN have a history of other diseases including eczema (atopic dermatitis), diabetes, lymphoma, HIV infection, severe anemia, or kidney disease. The exact cause of PN was not previously well-understood. It was thought that nodules were more likely to form when skin has been scratched or irritated in some way. Therefore, the act of a person scratching skin can cause the nodules to form. However, the cause of the skin to originally become intensely itchy was unclear.

Roughly 50% of people with PN have a history of atopy. The main symptom of PN is the formation of hard, very itchy lumps (nodules) on the skin. The nodules can range in size from very small to about half an inch in diameter. The nodules often have a rough, dry top and can range in number from a few to hundreds. Nodules most commonly form on the outer arms, shoulders, and legs. Nodules can also form on the neck and trunk, and they rarely form on the face and palms. They may be lighter or darker in color than the surrounding skin. Scarring may occur after nodules begin to heal. The symptoms of PN can begin at any age but are most common in adults after 50 years. People who have PN may become very concerned about the appearance of the nodules, and the intensely itchy skin may interfere with sleep or with everyday activities. This can cause people with PN to develop stress and depression.

Pruritus refers to itchy skin and/or an itch sensation. Pruritus may be caused by PN or other diseases or conditions such as dry skin. In some cases, pruritus involves generalized itchy skin over the whole body. In some cases, pruritus is localized to specific regions of the body such as on an arm or leg. Pruritus can be chronic or acute. Symptoms of pruritus include but are not limited to skin excoriations, redness, bumps, spots, blisters, dry skin, cracked skin, and leathery or scaly texture to the skin. In some cases, pruritus does not result in detectable changes to the skin. Behavioral responses to pruritus include but are not limited to skin scratching and/or skin massage. In some cases, skin scratching can result in excoriations that range from mild to severe. In some cases, patients with pruritus abstain from scratching and/or massaging the skin. Traditional treatments for PN include, but are not limited to, skin moisturizers, topical emollients, antihistamines such as diphenhydramine, topical corticosteroids, topical calcineurin inhibitors, and phototherapy. With narrow banded UVB and systemic immunosuppressive drugs like cyclosporine or methotrexate.

The present disclosure for the first time illuminates underlying gene expression patterns that are associates with PN and may be used to diagnose PN, identify a subject that is likely to respond to treatment (such as treatment with an anti-IL-31RA antibody), and determine whether a subject is properly responding to a treatment.

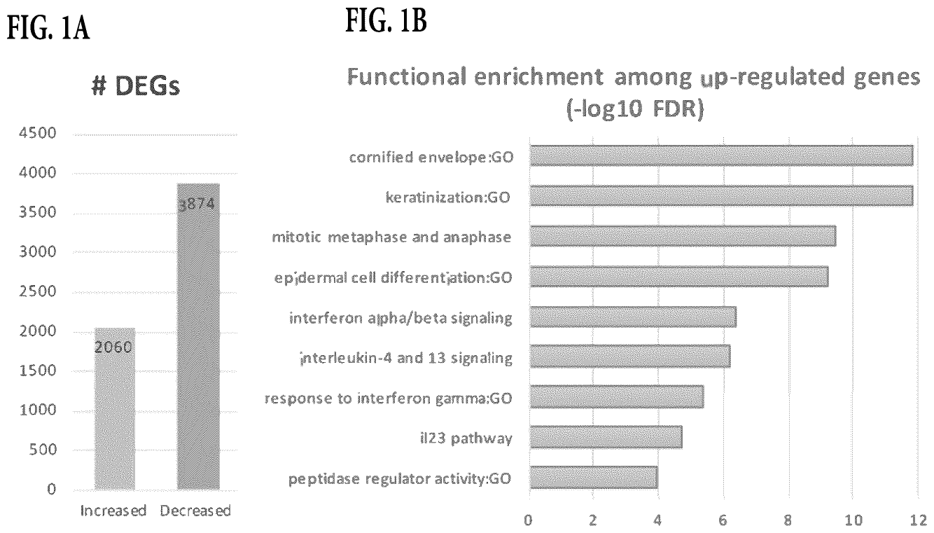

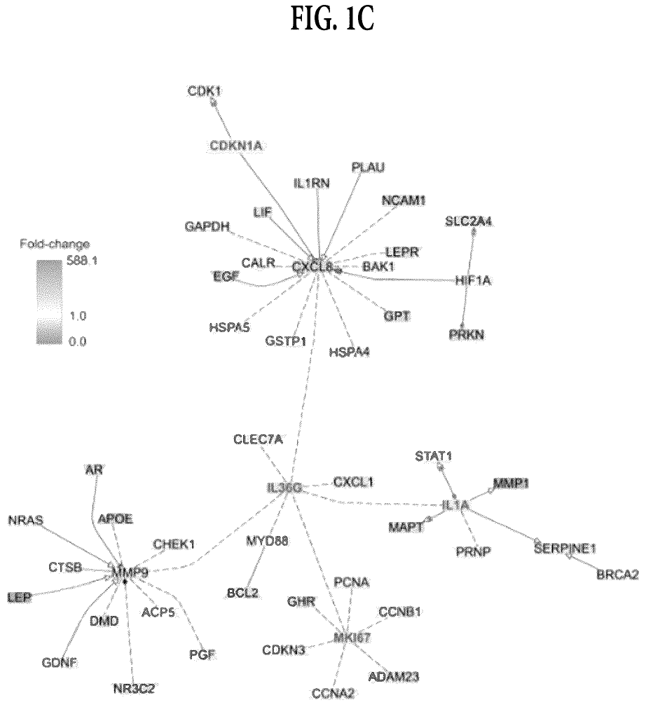

In particular, the present disclosure shows that a subject with PN may differentially express at least 5,934 genes (known as differentially expressed genes or DEGs), which are shown in FIG. 1A and Table 1 below. This differential gene expression may be observed in the skin of the subject and, in particular, in a skin sample comprising or consisting of a nodule or lesion. In some embodiments, at least 1, at least 2, at least 3, at least 4, at least 5, at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, or at least 2500 and up to 2500, 3000, 3500, 4000, 4500, 5000, 5500, or about 6000 of the disclosed DEGs may be differentially expressed in a subject with PN. Of these DEG, 2,060 may be increased (i.e., overexpressed) and 3,874 may be decreased (i.e., under expressed). Genes that may be increased the most include:
KRT6C, which may be increased at least 100-fold, at least 150 fold, at least 200-fold, at least 250-fold, at least 300-fold, at least 350-fold, at least 400-fold, at least 450-fold, at least 500-fold, at least 550-fold, or 588-fold compared to the expression level in a sample (e.g., a skin sample) from an individual that does not have PN;
DEFB4A, which may be increased at least 25-fold, at least 50-fold, at least 75-fold, at least 100-fold, at least 125-fold, or at least 150 fold compared to the expression level in a sample (e.g., a skin sample) from an individual that does not have PN; and
KRT16, which may be increased at least 10-fold, at least 20-fold, at least 30-fold, at least 40-fold, at least 50-fold, at least 60-fold, at least 70-fold, at least 80-fold, or at least 90 fold compared to the expression level in a sample (e.g., a skin sample) from an individual that does not have PN.

Decreased genes include:
LCE5A, which may be decreased at least 2-fold, at least 3-fold, at least 4-fold, at least 5-fold, at least 6-fold, at least 7-fold, at least 8-fold, at least 9-fold, at least 10-fold, or at least 11 fold compared to the expression level in a sample (e.g., a skin sample) from an individual that does not have PN; and
AQP7, which may be decreased at least 2-fold, at least 3-fold, at least 4-fold, at least 5-fold, at least 6-fold, at least 7-fold, or at least 7.9 fold compared to the expression level in a sample (e.g., a skin sample) from an individual that does not have PN.

Genes that encode cytokines may also be overexpressed in subjects suffering from PN. For the cytokine genes, the most prominent up-regulated genes are IL-36 family members and IL-20 family members. These upregulated or overexpressed genes may include:
IL36A (e.g., about 6.8-fold, FDR=1.8×10⁻⁴);
IL36G (e.g., about 8.4-fold, FDR=3.9×10⁻²⁵);
IL19 (e.g., about 5.1-fold, FDR=7.4×10⁻⁴);
IL20 (e.g., about 3.5-fold, FDR=1.7×10⁻³);
IL22 (e.g., about 2.7-fold, FDR=2.9×10⁻²);
IL24 (e.g., about 5.8-fold, FDR=3.8×10⁻¹⁰); and
IL26 (e.g., about 4.9-fold, FDR=3.3×10⁻³).
   Each of these IL-36 and IL-20 family member cytokine genes may be overexpressed by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, or at least about 8-5-fold compared to the expression level in a sample (e.g., a skin sample) from an individual that does not have PN.

Other factors that may be upregulated or overexpressed include ILIA (e.g., about 4.7-fold, FDR=1.0×10⁻¹²) and IL1B (e.g., about 4.1-fold, FDR=3.7×10⁻⁶). Additionally, the IL4R gene may be increased by e.g., about 2.6-fold (FDR=6.3×10⁻¹⁹). Table 1 at the end of the examples section of the specification provides a more exhaustive list of the DEGs.

In addition to the foregoing genes, the present disclosure also shows that certain plasma markers or signatures may be altered as a result of successful treatment of PN with an anti-IL-31RA antibody, such as nemolizumab. Such markers or signatures are detectable by, for example, by mass spectrometry and other methods of protein assessment (e.g., ELISA, Western blot, etc.). Circulating plasma protein markers or signatures that may be modulated as a result of treatment with an anti-IL-31RA antibody, such as nemolizumab, can include leukocyte migration and cell movement, the IL-6 pathway, the vascular endothelial growth factor (VEGF) pathway, the STAT3 (signal transducer and activator of transcription 3) pathway, the STAT5b (signal transducer and activator of transcription 5b) pathway, the TGFB1 (transforming growth factor beta-1) pathway, and neuronal ontologies.

The STAT3 pathway, a direct target of IL-31 signaling is also inhibited in the disclosed nemolizumab responder signature, this suggesting target engagement. STAT3 activity and expression may be comparatively high in a subject with PN relative to an individual or population without PN or in the subject prior to commencing treatment with an anti-IL-31RA antibody, such as nemolizumab.

The amount of circulating pro-inflammatory cytokines may also be comparatively high in a subject with PN relative to an individual or population without PN or in the subject prior to commencing treatment with an anti-IL-31RA antibody, such as nemolizumab. Such pro-inflammatory cytokine signatures can include, but are not limited to, IL-6 and VEGF. In a PN subject that receives treatment with an anti-IL-31RA antibody, such as nemolizumab, the treatment may result in a decrease in one or both of IL-6 and VEGF, or decreases or inhibition of the IL-6, VEGF, or both signaling pathways relative to a baseline level. The baseline level may be determined relative to (i) a control sample obtained from an individual or individuals (i.e., a population) without PN or (ii) a biological sample obtained from the subject prior to administration of the anti-IL-31RA antibody.

TGFB1 activity is also inhibited in the disclosed nemolizumab responder signature. TGFB1 activity and expression may be comparatively high in a subject with PN relative to an individual or population without PN or in the subject prior to commencing treatment with an anti-IL-31RA antibody, such as nemolizumab.

For the purposes of the disclosed plasma protein markers, the amount of the disclosed protein markers in the plasma of a subject with PN may be at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold higher than a baseline level. The baseline level may be determined relative to (i) a control sample obtained from an individual or individuals (i.e., a population) without PN or (ii) a biological sample obtained from the subject prior to administration of the anti-IL-31RA antibody. Similarly, after a subject with PN is treated with an anti-IL-31RA antibody, such as nemolizumab (e.g., 2 weeks, 4 weeks, 6 weeks, 8 weeks, 10 weeks, or 12 weeks after administration of the antibody), the amount of the disclosed plasma protein markers in the subject may decrease at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold relative to a baseline level. The baseline level may be determined relative to (i) a control sample obtained from an individual or individuals (i.e., a population) without PN or (ii) a biological sample obtained from the subject prior to administration of the anti-IL-31RA antibody.

Additionally, neuronal ontologies (e.g., CREB signaling in neurons, Synaptogenesis signaling pathway, Cell death of neuroglia and Apoptosis of neuroglia) may be upregulated in subject with PN, and subsequently downregulated in nemolizumab responders subjects, thus emphasizing the impact of IL-31 as a neuro-inflammatory cytokine in PN.

Thus, the present disclosure provides methods of diagnosing PN, comprising detecting in a sample obtained from a subject suspected of having PN the expression level of at least one, at least two, at least three, at least four, or at least five of the differentially expressed genes (DEGs) in Table 1 (e.g., KRT6C, DEFB4A, KRT16, LCE5A, AQP7, IL-36 family members, IL-20 family members, etc.) and comparing the expression level of the DEGs to a reference level, which may be based on the gene expression level in a sample (e.g., a skin sample) from an individual that does not have PN. In some embodiments, the sample obtained from the subject suspected of having PN is a skin sample, which may comprise a lesion or a nodule. If certain genes are upregulated or overexpressed (e.g., KRT6C, DEFB4A, KRT16) and/or if other genes are down regulated or under expressed (e.g., LCE5A, AQP7) then the subject may be diagnosed with PN.

The present disclosure also provides methods of determining the likelihood of a positive response to treatment (e.g., treatment with an anti-IL-31RA antibody, such as nemolizumab) in a subject with PN, comprising detecting in a sample obtained from a subject with PN the expression level of at least one, at least two, at least three, at least four, or at least five of the differentially expressed genes (DEGs) in Table 1 (e.g., KRT6C, DEFB4A, KRT16, LCE5A, AQP7, IL-36 family members, IL-20 family members, etc.) and comparing the expression level of the DEGs to a reference level, which may be based on the gene expression level in a sample (e.g., a skin sample) from an individual that does not have PN. In some embodiments, the sample obtained from the subject with PN is a skin sample, which may comprise a lesion or a nodule. If certain genes are upregulated or overexpressed (e.g., KRT6C, DEFB4A, KRT16) and/or if other genes are down regulated or under expressed (e.g., LCE5A, AQP7) then the subject is likely to respond to the treatment.

The present disclosure also provides methods of determining whether a subject with PN is responding to treatment (e.g., treatment with an anti-IL-31RA antibody, such as nemolizumab), comprising detecting in a sample obtained from a subject with PN the expression level of at least one, at least two, at least three, at least four, or at least five of the differentially expressed genes (DEGs) in Table 1 (e.g., KRT6C, DEFB4A, KRT16, LCE5A, AQP7, IL-36 family members, IL-20 family members, etc.) and comparing the expression level of the DEGs to a baseline level of expression, wherein baseline level of gene expression is from a sample (e.g., a skin sample) from the same individual before treatment was commenced. In some embodiments, the samples may be skin samples, which may comprise a lesion or a nodule. If the expression levels of certain genes decreases (e.g., KRT6C, DEFB4A, KRT16) and/or if the expression level of other genes increases (e.g., LCE5A, AQP7) then the subject is responding to the treatment.

The present disclosure also provides methods of determining whether a subject with PN is responding to treatment (e.g., treatment with an anti-IL-31RA antibody, such as nemolizumab), comprising detecting in a post-treatment plasma sample obtained from a subject with PN that has been administered at least one dose of an anti-IL-31RA antibody one or more biomarkers selected from leukocyte migration and cell movement, the IL-6 pathway, the VEGF pathway, the STAT3 pathway, the STAT5b pathway, the TGFB1 pathway, and the disclosed neuronal ontologies; wherein a decrease in leukocyte migration and cell movement, a decrease in IL-6 or a decrease in IL-6 pathway signaling, a decrease in VEGF or a decrease in VEGF pathway signaling, a decrease in STAT3 or a decrease in STAT3 pathway signaling, a decrease in STAT5b or a decrease in STAT5b pathway signaling, a decrease in TGFB1 or a decrease in TGFB1 pathway signaling, or an increase in the disclosed neuronal ontologies relative to a baseline amount indicates responsiveness to treatment, wherein the baseline amount was determined from a plasma sample obtained from the same subject before treatment was commenced.

The present disclosure also defines for the first time the biological processes that are enriched in PN skin gene ontology (GO). The most prominent GO categories associated with PN are:
“cornified envelope” (FDR=1.5×10⁻¹²),
“epidermal cell differentiation” (FDR=6.4×10⁻¹⁰),
“keratinization” (FDR=1.6×10⁻¹²),
“peptidase regulator activity” (FDR=1.1×10⁻⁴),
“interleukin-4 and 13 signaling” (FDR=6.8×10⁻⁷),
“interferon alpha beta signaling” and “response to interferon gamma” (FDR=4.1×10⁻⁷, and FDR=4.1×10⁻⁶, respectively),
“IL23 pathway” (FDR=2×10⁻⁵), and
“mitotic metaphase and anaphase” (FDR=3.8×10⁻¹⁰).

These GO categories are defined in more detail by FIG. 1B and Table 2 at the end of the examples section of the specification. These categories reflect the hyperproliferative nature of PN, which was discovered to be associated with altered epidermal differentiation and inflammatory components. For example, the proliferative marker Ki67 (MKI67), the cell cycle gene CDKN1A, and inflammatory networks involving IL-1 and IL-36 have now been revealed as potentially associated with PN pathogenesis as shown in FIG. 1C.

The present disclosure also discloses 20 co-expression modules that were identified in non-lesional skin and 10 clusters in lesional PN skin, as shown in FIG. 1D and Table 3 at the end of the examples section of the specification. Distinct functions could be defined for these co-expressing gene modules, especially for the co-expression modules in PN lesional skin, with the most prominent involved immunological processes (module #8) including “immune-response” (FDR=1.8×10⁻⁴⁷), “defense response” (FDR=1.2×10⁻³⁹); cell proliferation (module #6) including “cell cycle” (FDR=2.9×10⁻⁹⁴), “DNA metabolic process” (FDR=8.7×10⁻⁶⁷); and epidermal processes (module #5), such as “epidermis development” (FDR 3.5×10⁻¹⁰), “keratinization” (FDR=1.7×10⁻⁶). See also FIG. 1E. Other notable findings were changes in “extracellular matrix” (FDR=1.16×10⁻⁵⁹; module #2) and included genes such as MMP14, MMP16, COL1A1, COL1A2, and COL3A1, which were modestly elevated (FC≥1.4; FDR≤6×10⁻²) in the lesional skin, consistent with the association of PN with skin fibrosis

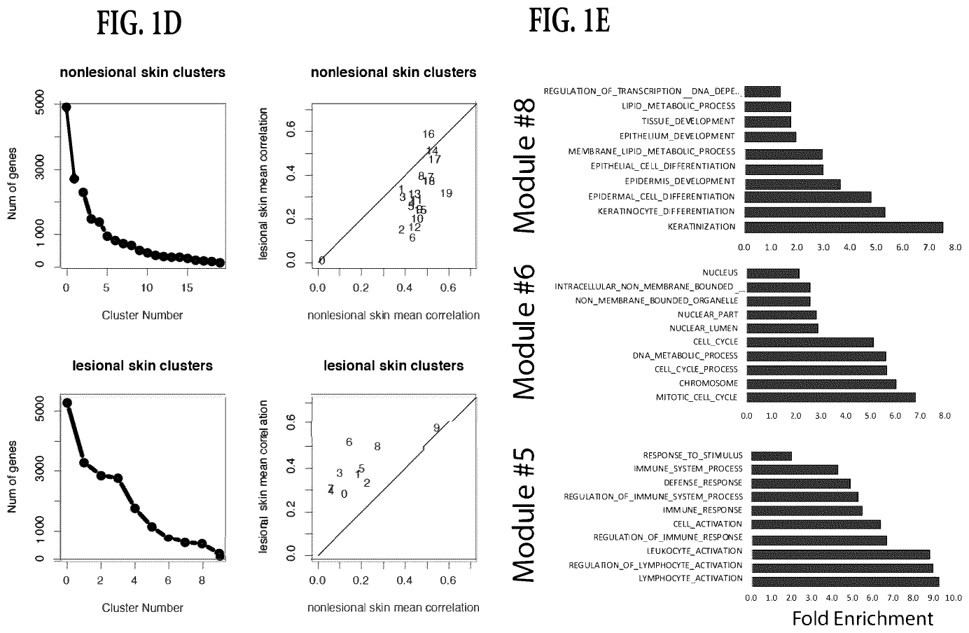

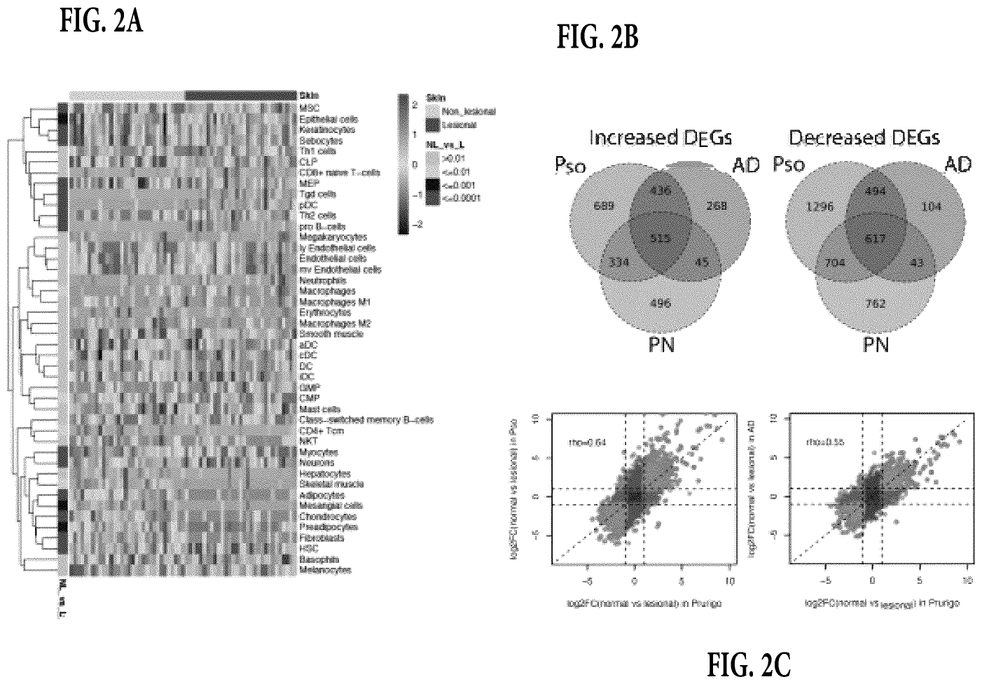

The present disclosure also provides specific cell-type signatures for non-lesional and lesional PN skin samples. Enrichment for transcriptomic signature associated with epithelial cells and keratinocytes was observed (p<0.001 and p<0.0001, respectively), as shown in FIG. 2A. A subject with PN may show a Th2 associated signature (p<0.0001), consistent with the enriched GO categories for IL-4/IL-13 (as discussed above and shown FIG. 1). Other inflammatory signatures, such as macrophages (p<0.01), may be more variable.

As shown in FIG. 2B, a 3-way comparison of the PN transcriptome with that of atopic dermatitis (AD) and Psoriasis indicated that all three diseases share significant overlap for both up-regulated and down-regulated genes. However, the correlation of the effect sizes in the lesional skin was more pronounced between PN and psoriasis (spearman correlation p=0.64) than between PN and AD (p=0.55). Genes that are commonly up-regulated in both psoriasis and PN include those participating in cytokine activity (CCL3, CXCL10, IFNG, IL12B, IL19, IL1B, IL20, etc.) and keratinization (KRT16, KRT17, LCE3A, LCE3E, etc.) (Table 4).

Thus, the present disclosure provides methods of identifying or diagnosing PN based on gene ontology, co-expression module, and/or gene signature. For example, a subject may present with a PN skin gene ontology (GO) shown in FIG. 1B or Table 2. Additionally or alternatively, the skin sample may also show upregulation or overexpression of Ki67 (MKI67), CDKN1A, and/or inflammatory networks involving IL-1 and IL-36. Additionally or alternatively, a subject may present with a co-expression module as shown in FIG. 1D or Table 3. Additionally or alternatively, a subject with PN may present (e.g., in a skin sample) with the transcriptomic or Th2 signature disclosed in FIG. 2A.

Additionally, the present disclosure provide an unprecedented insight into the pathogenesis of PN, and the associated tissue-specific and cell-type specific changes that occur in PN skin both during the development of the disease and in response to treatment with an anti-IL31RA antibody. Changes in PN skin can be observed across both immune and stromal cell populations, including keratinocytes, endothelial cells, and most profoundly fibroblasts and fibroblast subpopulations, during treatment, as PN pathogenesis is characterized by increased profibrotic responses accompanied by an immune shift away from IL-13 and IL-22 responses.

A characteristic histopathologic feature of PN is fibrosis of the papillary dermis with vertically arranged collagen fibers, including a marked increase in dense collagen in the papillary dermis in lesional PN skin by trichrome staining and increased expression of pro-collagen I in the papillary dermis. Moreover, COL11A1+ fibroblasts can be major source of activated and enriched profibrotic responses, including increased mRNA expression of both collagen I and collagen III. Consistent with their profibrotic function, COL11A11+ fibroblasts may be primarily found in the papillary dermis, where the fibrotic response is most marked with either trichrome or pro-collagen I staining indicative of active collagen I biosynthesis.

Expansion of the COL11A1+ fibroblast subpopulation is unique to PN and not seen in atopic dermatitis (AD) skin. Furthermore, the profibrotic effect of this population is not observed in AD COL11A11+ fibroblasts. Of the two components of the heterodimeric IL-31 receptor, IL31RA expression was detected on both keratinocytes and fibroblasts, whereas OSMRB expression was found to be more widespread in different cell populations. Accordingly, the two key cell types responding to IL-31 in PN are likely fibroblasts and keratinocytes, which is in keeping with the observation of the transcriptomic shifts driven by treatment with an anti-IL-31RA antibody (e.g., nemolizumab) that can be attributed to those two cell types.

Other cell types including endothelial cells and pericytes also contribute to fibrosis in leshional prurigo nodularis (LPN) skin. Endothelial changes are known in LPN, but the nature of these shifts has not been previously detailed. The present disclosure shows that endothelial cells, likely under the action of proinflammatory and profibrotic cytokines such as TGFB, contribute to extracellular matrix reorganization. TGFβ may be an upstream promoter of fibrosis in PN, as TGFβ was observed being expressed in a wide range of cell types in PN skin including endothelial cells, fibroblasts, and nerve cells for TGFB1, and fibroblasts and pericytes for TGFB2 and TGFB3. Notably, TGFB2 and TGFB3 have been more strongly implicated in fibrosis than TGFB1.

One of the most characteristic histologic features of PN is the presence of compact orthohyperkeratosis, with irregular epidermal hyperplasia. Keratinocytes demonstrate marked transcriptomic changes in lesional PN skin with the most marked shifts seen in inflammatory keratinocytes, defined by KRT6, KRT16, KRT17, S100A8 and S100A9 expression, and with this analyses indicating a key role of Th2 cytokines (e.g., IL13 and IL22) in this transition. The most enriched biological categories in the inflammatory keratinocyte subset had to do with mitochondrial function and protein translation suggesting the generation of reactive oxygen species and cellular stress that can contribute to inflammatory responses in the skin.

Immune cell infiltration also occurs in lesional PN skin and is characterized by shifts in specific immune cell populations. The most pronounced shifts can be observed in macrophage populations, particularly lipid-associated macrophages characterized by the expression of APOE and TREM2. The lipid metabolic products from lipid-associated macrophages have been shown to trigger the production of proinflammatory cytokines in atherosclerosis, which in turn amplify inflammatory responses. T cells were also prominent in PN lesions, with an increased number of cycling T cells, as well as NK, CD8⁺, and Tregs. Furthermore, various stromal cell populations, particularly endothelial and pericytes, had increased expression of various proinflammatory cytokines, chemokines, and adhesion molecules, indicating a likely role in immune trafficking and immune amplification in PN. This includes increased expression of the adhesion molecule ICAM1, E-selectin (SELE), and IL6 in endothelial cells, and CCL2, CCL3, CCL4, CCL13, CCL18, CXCL2, and CXCL12 that were expressed by various cell types (e.g., myeloid cells, pericytes, and endothelial cells) in PN skin. CCL2 and IL-6 have established roles in the development of fibrosis. CCL2 is the most potent profibrotic chemokine; through CCR2, CCL2 acts directly on fibroblasts stimulating collagen synthesis. Likewise, IL-6 trans-signaling enhances lung fibroblast proliferation and extracellular matrix protein production.

The present disclosure shows that shifts in cell populations in the epidermis are highly similar between both PN and AD with both diseases having a prominent “inflammatory” keratinocyte subset characterized by the expression of pro-inflammatory molecules including S100A8 and S100A9 along with increased expression of the inflammatory keratins KRT6 and KRT16. Changes in the expression of S100A8, S100A9, and KRT16 have been described in AD skin, but their expression has not been addressed in PN skin. These epidermal changes are accompanied by subtle changes in the gene expression in PN vs. AD skin, with immune-related processes such as antimicrobial responses, and regulators of T cell trafficking only observed in LAD, but not LPN keratinocytes.

Changes in T cell phenotype are seen between LPN and LAD skin, particularly within the CD4 effector T cell population, where mRNA expression of IL13 and IL22 was markedly lower in LPN skin compared to LAD skin. IL-22 is known to promote epidermal proliferation and activate innate immune and antimicrobial responses in the skin. Thus, while PN is an inflammatory-driven disease, it may not be centered around IL-13/IL-22 responses to the same degree as AD.

The present disclosure provides mechanisms of action of IL-31 receptor antagonism. In particular, transcriptomic shifts indicating stabilization of extracellular matrix remodeling and normalization of epidermal differentiation can be seen as a result of the disclosed methods of treatment. Normalization of the pathologic transcriptomic signatures observed in the COL11A1+ fibroblasts and the inflammatory keratinocyte subset may be linked to clinical improvement of PN skin lesions during treatment, which may also reverse the cellular, genetic, and molecular underpinnings of PN development and progression.

Thus, the present disclosure provides methods of deactivating, decreasing activation, or decreasing a number of COL11A1+ fibroblasts in a subject with PN, comprising administering to the subject an anti-IL-31RA antibody, wherein administering the anti-IL-31RA antibody results in deactivation, a decrease in activation, or a decrease in number of COL11A1+ fibroblasts in the subject's skin. In some embodiments, the COL11A1+ fibroblasts are found in the papillary dermis. Such a decrease inactivation or total number of activated fibroblast may be determined relative to the number and activation status of fibroblasts in a skin lesion prior to treatment with an anti-IL-31RA antibody (e.g., nemolizumab).

The present disclosure also provides methods of decreasing TGFβ expression in at least one cell type in a subject with PN, comprising administering to the subject an anti-IL-31RA antibody, wherein administering the anti-IL-31RA antibody results in a decrease in TGFβ expression in at least one cell type in the subject's skin. In some embodiments, the at least one cell type comprises fibroblasts, endothelial cells, pericytes, nerve cells, or any combination thereof. In some embodiments, the decrease in TGFβ expression comprises a decrease in the expression of TGFB1, TGFB2, TGFB3, or any combination thereof. Such a decrease in TGFβ expression may be determined relative to the level of expression in a corresponding cell type in a PN skin lesion prior to treatment with an anti-IL-31RA antibody (e.g., nemolizumab).

The present disclosure provides methods of decreasing expression at least one inflammatory gene expressed by a keratinocyte in a subject with PN, comprising administering to the subject an anti-IL-31RA antibody, wherein administering the anti-IL-31RA antibody results in a decrease in at least one inflammatory gene expressed by a keratinocyte in the subject's skin. In some embodiments, the at least one inflammatory gene is selected from KRT6, KRT16, KRT17, S100A8, S100A9, and any combination thereof. In some embodiments, the keratinocyte expresses Th2 cytokines. In some embodiments, administering the anti-IL-31RA antibody results in a decrease in reactive oxygen species and/or cellular stress to which the keratinocyte is exposed. Such a decrease in inflammatory gene expression may be determined relative to the level of expression in a corresponding cell type in a PN skin lesion prior to treatment with an anti-IL-31RA antibody (e.g., nemolizumab).

The present disclosure provides methods of decreasing infiltration of at least one type of immune cell in a skin lesion of a subject with PN, comprising administering to the subject an anti-IL-31RA antibody, wherein administering the anti-IL-31RA antibody results in a decrease in infiltration of at least one type of immune cell in at least one lesion in the subject's skin. In some embodiments, the at least one type of immune cell comprises a macrophage. In some embodiments, the macrophage is a lipid-associated macrophage characterized by expression of APOE and TREM2. In some embodiments, the at least one type of immune cell comprises T cells, NK cells, CD8⁺ cells, Tregs and any combination thereof. In some embodiments, includes administering the anti-IL-31RA antibody results in a decrease in expression of ICAM1, E-selectin (SELE), IL6 CCL2, CCL3, CCL4, CCL13, CCL18, CXCL2, CXCL12, and any combination thereof in at least one cell type in the lesion. In some embodiments, the at least on cell type in the lesion comprises myeloid cells, pericytes, endothelial cells, and any combination thereof. Such a decrease in immune cell infiltration or expression of ICAM1, E-selectin (SELE), IL6 CCL2, CCL3, CCL4, CCL13, CCL18, or CXCL2, CXCL12 may be determined relative to the amount of infiltration observed in a skin lesion prior to treatment of relative to the level of expression in a corresponding cell type in a PN skin lesion prior to treatment with an anti-IL-31RA antibody (e.g., nemolizumab).

The expression levels of the genes and markers disclosed herein can be determined by any suitable methods known in the art, including but not limited to RT-qPCR, RT-PCR, RNA-seq, Northern blotting, Serial Analysis of Gene Expression (SAGE), DNA or RNA microarrays, and in situ hybridization. At the protein level, the disclosed biomarkers may be detected or measured using, for example, Western blotting, ELISA (Enzyme-Linked ImmunoSorbent Assay), surface plasmon resonance, and mass spectrometry.

Subjects with PN or suspected of having PN that present with any of the disclosed DEGs, gene ontologies, co-expression modules, or gene signatures are suitable for treatment or preventions with an anti-IL-31RA antibody, such as nemolizumab, as described in further detail herein.

The present disclosure provides a combination of (1) a pharmaceutical composition for use in the treatment or prevention of prurigo nodularis (PN), comprising an anti-IL31RA antibody (e.g., nemolizumab or a fragment or variant thereof), as an active ingredient; and (2) a diagnostic agent which detects in a subject suspected of having PN the expression level of at least one gene selected from the genes disclosed in Table 1 compared to a reference level of expression for the at least one gene.

### III. THERAPEUTIC ANTIBODIES AND INTERLEUKIN 31 RECEPTOR SUBUNIT ALPHA (IL-31RA)

Interleukin-31 (IL-31) is a neuro-inflammatory cytokine that could activate both structural, immune cells and peripheral nerves. It has been involved in a number of chronic inflammatory diseases, including atopic dermatitis. IL-31 is produced by a variety of cells, including type 2 helper (Th2) T-cells. IL-31 sends signals through a receptor complex made of IL— Interleukin 31 receptor subunit alpha (“IL-31RA,” also known as NR10, glm-r, and GPL) and oncostatin M receptor β (OSMRP) expressed in immune and epithelial cells, as well as in a subset of neurons.

IL-31RA forms a heterodimer with oncostatin M receptor (OSMR) when functioning as an IL-31 receptor. There are multiple known splicing variants of human-derived IL-31RA (WO 00/075314): NR10.1 consists of 662 amino acids and contains a transmembrane domain. NR10.2 is a soluble receptor-like protein consisting of 252 amino acids without the transmembrane domain. Further, known IL-31RA splicing variants that function as transmembrane receptor proteins include NR10.3 and IL-31RAv3. Preferred IL-31RA variants include NR10.3 (also referred to as ILRAv4 (Nat Immunol 5, 752-60, 2004) and IL-31RAv3. NR 10.3 (IL31RAv4) consists of 662 amino acids (WO 00/075314; Nat Immunol 5, 752-60, 2004) and IL31RAv3 consists of 732 amino acids (GenBank Accession No: NM-139017).

The amino acid sequence of IL31RAv4 is:

(SEQ ID NO: 1)
MKLSPQPSCVNLGMMWTWALWMLPSLCKFSLAALPAKPEN
ISCVYYYRKNLTCTWSPGKETSYTQYTVKRTYAFGEKHDN
CTTNSSTSENRASCSFFLPRITIPDNYTIEVEAENGDGVI
KSHMTYWRLENIAKTEPPKIFRVKPVLGIKRMIQIEWIK
PELAPVSSDLKYTLRFRTVNSTSWMEVNFAKNRKDKNQTY
NLTGLQPFTEYVIALRCAVKESKFWSDWSQEKMGMTEEEA
PCGLELWRVLKPAEADGRRPVRLLWKKARGAPVLEKTLGY
NIWYYPESNTNLTETMNTTNQQLELHLGGESFWVSMISYN
SLGKSPVATLRIPAIQEKSFQCIEVMQACVAEDQLVVKWQ
SSALDVNTWMIEWFPDVDSEPTTLSWESVSQATNWTIQQD
KLKPFWCYNISVYPMLHDKVGEPYSIQAYAKEGVPSEGPE
TKVENIGVKTVTITWKEIPKSERKGIICNYTIFYQAEGGK
GFSKTVNSSILQYGLESLKRKTSYIVQVMASTSAGGTNGT
SINFKTLSFSVFEIILITSLIGGGLLILIILTVAYGLKKP
NKLTHLCWPTVPNPAESSIATWHGDDFKDKLNLKESDDSV
NTEDRILKPCSTPSDKLVIDKLVVNFGNVLQEIFTDEART
GQENNLGGEKNGTRILSSCPTSI

The amino acid sequence of IL31RAv3 is:

(SEQ ID NO: 2)
MMWTWALWMLPSLCKFSLAALPAKPENISCVYYYRKNLTC
TWSPGKETSYTQYTVKRTYAFGEKHDNCTTNSSTSENRAS
CSFFLPRITIPDNYTIEVEAENGDGVIKSHMTYWRLENIA
KTEPPKIFRVKPVLGIKRMIQIEWIKPELAPVSSDLKYTL
RFRTVNSTSWMEVNFAKNRKDKNQTYNLTGLQPFTEYVIA
LRCAVKESKFWSDWSQEKMGMTEEEAPCGLELWRVLKPAE
ADGRRPVRLLWKKARGAPVLEKTLGYNIWYYPESNTNLTE
TMNTTNQQLELHLGGESFWVSMISYNSLGKSPVATLRIPA
IQEKSFQCIEVMQACVAEDQLVVKWQSSALDVNTWMIEWF
PDVDSEPTTLSWESVSQATNWTIQQDKLKPFWCYNISVYP
MLHDKVGEPYSIQAYAKEGVPSEGPETKVENIGVKTVTIT
WKEIPKSERKGIICNYTIFYQAEGGKGFSKTVNSSILQYG
LESLKRKTSYIVQVMASTSAGGINGTSINFKTLSFSVFEI
ILITSLIGGGLLILIILTVAYGLKKPNKLTHLCWPTVPNP
AESSIATWHGDDFKDKLNLKESDDSVNTEDRILKPCSTPS
DKLVIDKLVVNFGNVLQEIFTDEARTGQENNLGGEKNGYV
TCPFRPDCPLGKSFEELPVSPEIPPRKSQYLRSRMPEGTR
PEAKEQLLFSGQSLVPDHLCEEGAPNPYLKNSVTAREFLV
SEKLPEHTKGEV

Mouse-derived IL-31RA comprises the amino acid sequence:

(SEQ ID NO: 3)
MWTLALWAFSFLCKFSLAVLPTKPENISCVFYFDRNLTCT
WRPEKETNDTSYIVTLTYSYGKSNYSDNATEASYSFPRSC
AMPPDICSVEVQAQNGDGKVKSDITYWHLISIAKTEPPII
LSVNPICNRMFQIQWKPREKTRGFPLVCMLRFRTVNSSRW
TEVNFENCKQVCNLTGLQAFTEYVLALRFRFNDSRYWSKW
SKEETRVTMEEVPHVLDLWRILEPADMNGDRKVRLLWKK
ARGAPVLEKTFGYHIQYFAENSTNLTEINNITTQQYELLL
MSQAHSVSVTSENSLGKSQEAILRIPDVHEKTFQYIKSMK
AYIAEPLLVVNWQSSIPAVDTWIVEWLPEAAMSKFPALS
WESVSQVTNWTIEQDKLKPFTCYNISVYPVLGHRVGEPYS
IQAYAKEGTPLKGPETRVENIGLRTATITWKEIPKSARNG
FINNYTVFYQAEGGKELSKTVNSHALQCDLESLTRRTSYT
VWVMASTRAGGTNGVRINFKTLSISVFEIVLLTSLVGGGL
LLLSIKTVTFGLRKPNRLTPLCCPDVPNPAESSLATWLGD
GFKKSNMKETGNSGDTEDVVLKPCPVPADLIDKLVVNFEN
FLEVVLTEEAGKGQASILGGEANEYVTSPSRPDGPPGKSF
KEPSVLTEVASEDSHSTCSRMADEAYSELARQPSSSCQSP
GLSPPREDQAQNPYLKNSVTTREFLVHENIPEHSKGEV

Cynomolgus monkey-derived IL-31RA comprises the amino acid sequence:

(SEQ ID NO: 4)
MMWTWALWMFPLLCKFGLAALPAKPENISCVYYYRKNLTC
TWSPGKETSYTQYTAKRTYAFGKKHDNCTTSSSTSENRAS
CSFFLPRITIPDNYTIEVEAENGDGVIKSDMTCWRLEDIA
KTEPPEIFSVKPVLGIKRMIRIEWIKPELAPVSSDLKYAL
RFRTVNSTSWMEVNFAKNRKDTNQTYNLMGLQAFTEYVVA
LRCAVKESKFWSDWSQEKMGMTEEEAPCGLELWRVLKPTE
VDGRRPVRLLWKKARGAPVLEKTLGYNIWYFPENNTNLTE
TVNTTNQQLELHLGGESYWVSMISYNSLGKSPVTTLRIPA
IQEKSFRCIEVMQACLAEDQLVVKWQSSALDVNTWMIEWF
PDMDSEHPTLSWESVSQATNWTIQQDKLKPFWCYNISVYP
MLHDKVGEPYSIQAYAKEGIPSKGPETKVENIGVKTVTIT
WKEIPKSERKGIICNYTIFYQAEGGKGFSKTVNSSILQYG
LESLKRKTSYTVRVMASTSAGGINGTSINFKTLSFSVFEI
ILITSLIGGGLLILIILTVAYGLKKPNKLTHLCWPSVPNP
AESSIATWRGDDFKDKLNLKESDDSVNTEDRILKPCSTPS
DKLVIDKSVVNFGNVLQEMFTDEARTGQENNLGGEKNEY
VTHPFRADCPLGKSFEELPVSPEIPPRKSQYLRSRMPEGT
CLEAEEQLLVSGQSLESLAPDHVREAAAPNPYLKNSVTTR
EFLVSQKLPEHTKGEV

For the purposes of the present disclosure, an anti-IL-31RA antibody (i.e., a therapeutic antibody), such as nemolizumab, must bind to at least human IL-31RA or a splice variant thereof.

As used herein, the term “antibody” collectively refers to immunoglobulins or immunoglobulin-like molecules including IgA, IgD, IgE, IgG and IgM, combinations thereof or fragments thereof. Fragments of antibodies may include, for example, Fab fragments and single chain variable fragments (scFv). An antibody generally comprises heavy (H) chains and light (L) chains interconnected by disulfide bonds. There are two types of light chain, lambda (k) and kappa (x). There are five main heavy chain classes (or isotypes) which determine the functional activity of an antibody molecule: IgM, IgD, IgG, IgA and IgE. Each heavy and light chain contains a constant region and a variable region (also known as “domains”). In combination, the heavy and the light chain variable regions, also called the “Fab region,” specifically bind to a given antigen. Light and heavy chain variable regions contain a “framework” region interrupted by three hypervariable regions, also called “complementarity-determining regions” or “CDRs.” The extent of the framework region and CDRs has been defined (see Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

The CDRs are primarily responsible for binding to an epitope on an antigen. The CDRs of each chain are typically referred to as CDR1, CDR2, and CDR3, numbered sequentially starting from the N-terminus, and are also typically identified by the chain in which the particular CDR is located. Thus, a HCDR3 is located in the variable domain of the heavy chain of the antibody in which it is found, whereas a LCDR1 is the CDR1 from the variable domain of the light chain of the antibody in which it is found. An antibody that binds IL-31RA will have a specific V_{H} region and the V_{L} region sequence, and thus specific CDR sequences. Antibodies with different specificities generally have different CDRs. Although it is the CDRs that vary from antibody to antibody, only a limited number of amino acid positions within the CDRs are directly involved in antigen binding. These positions within the CDRs are called specificity determining residues (SDRs).

The Fc fragment region (Fc) of an antibody plays a role in modulating immune cell activity. The Fc region functions to guarantee that each antibody generates an appropriate immune response for a given antigen, by binding to a specific class of proteins found on certain cells, such as B lymphocytes, follicular dendritic cells, natural killer cells, macrophages, neutrophils, etc. and are called “Fc receptors.” Because the constant domains of the heavy chains make up the Fc region of an antibody, the classes of heavy chain in antibodies determine their class effects. The heavy chains in antibodies include alpha, gamma, delta, epsilon, and mu, and correlate to the antibody's isotypes IgA, IgG, IgD, IgE, and IgM, respectively. Thus, different isotypes of antibodies have different class effects due to their different Fc regions binding and activating different types of receptors.

There are four subclasses of IgG, which is the most abundant antibody isotype found in human serum. The four subclasses, IgG1, IgG2, IgG3, and IgG4, which are highly conserved. The amino acid sequence of the constant regions of these peptides are known in the art, e.g., see Rutishauser, U. et al. (1968) “Amino acid sequence of the Fc region of a human gamma G-immunoglobulin” PNAS 61(4):1414-1421; Shinoda et al. (1981) “Complete amino acid sequence of the Fc region of a human delta chain” PNAS 78(2):785-789; and Robinson et al. (1980) “Complete amino acid sequence of a mouse immunoglobulin alpha chain (MOPC 511)” PNAS 77(8):4909-4913.

All therapeutic antibodies, for the purposes of the disclosed methods and pharmaceutical uses, are antibodies or fragments thereof that bind to IL-31RA, but the specific anti-IL-31RA antibody is not limited. Nemolizumab is a preferred anti-IL-31RA antibody, but other anti-IL-31RA antibodies can be used as well. A therapeutic antibody suitable for use in the disclosed methods and pharmaceutical uses may be human, humanized, or chimeric, and it may be an IgA, IgG (i.e., IgG1, IgG2, IgG3, and IgG4), IgD, IgE, or IgM.

Nemolizumab is a humanized monoclonal antibody that binds to IL-31RA. Nemolizumab is annotated as follows: immunoglobulin G2-kappa, anti-[Homo sapiens IL31RA (interleukin 31 receptor subunit alpha)], humanized monoclonal antibody; gamma2 heavy chain (1-445) [humanized VH (Homo sapiens IGHV1-2*02 (83.70%)-(IGHD)-IGHJ5*01) [8.8.14] (1-121)-Homo sapiens IGHG2*01 (CH1 C10>S (135), R12>K (137), E16>G (141), S17>G (142) (122-219), hinge C4>S (223) (220-231), CH2 H30>Q (268) (232-340), CH3R 11>Q (355), Q98>E (419) (341-445)) (122-445)], (224-214′)-disulfide with kappa light chain (1′-214′) [humanized V-KAPPA (Homo sapiens IGKV1-39*01 (82.10%)-IGKJ4*01) [6.3.9] (1′-107′)-Homo sapiens IGKC*01 (108′-214′)]; dimer (227-227″:230-230″)-bisdisulfide. Nemolizumab has disulfide bridges at the following locations: Intra-H (C23-C104) 22-96 148-204 261-321 367-425 22″-96″ 148″-204″ 261″-321″ 367″-425″; Intra-L (C23-C104) 23′-88′ 134′-194′ 23′″-88′″ 134′″-194′″; Inter-H-L (h 5-CL 126) 224-214′ 224″-214′″; Inter-H-H (h 8, h 11) 227-227″ 230-230″. Nemolizumab has N-glycosylation sites at the following locations: H CH2 N84.4: 297, 297″. Nemolizumab lacks H chain C-terminal glycine and lysine (CHS G1>del, K2>del).

Nemolizumab comprises the following heavy chain amino acid sequence:

(SEQ ID NO: 5)
QVQLVQSGAEVKKPGASVKVSCKASGYTFTGYIMNWVRQA
PGQGLEWMGLINPYNGGTDYNPQFQDRVTITADKSTSTAY
MELSSLRSEDTAVYYCARDGYDDGPYTLETWGQGTLVTVS
SASTKGPSVFPLAPSSKSTSGGTAALGCLVKDYFPEPVTV
SWNSGALTSGVHTFPAVLQSSGLYSLSSVVTVPSSNFGTQ
TYTCNVDHKPSNTKVDKTVERKSCVECPPCPAPPVAGPSV
FLFPPKPKDTLMISRTPEVTCVVVDVSQEDPEVQFNWYVD
GVEVHNAKTKPREEQFNSTFRVVSVLTVVHQDWLNGKEYK
CKVSNKGLPAPIEKTISKTKGQPREPQVYTLPPSQEEMTK
NQVSLTCLVKGFYPSDIAVEWESNGQPENNYKTTPPMLDS
DGSFFLYSKLTVDKSRWQEGNVFSCSVMHEALHNHYTQKS
LSLSP.

Nemolizumab comprises the following light chain amino acid sequence:

(SEQ ID NO: 6)
DIQMTQSPSSLSASVGDRVTITCQASEDIYSFVAWYQQKP
GKAPKLLIYNAQTEAQGVPSRFSGSGSGTDFTLTISSLQP
EDFATYYCQHHYDSPLTFGGGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC.

The heavy chain variable region of nemolizumab comprises the amino acid sequence:

(SEQ ID NO: 7)
QVQLVQSGAEVKKPGASVKVSCKASGYTFTGYIMNWVRQA
PGQGLEWMGLINPYNGGTDYNPQFQDRVTITADKSTSTAY
MELSSLRSEDTAVYYCARDGYDDGPYTLETWGQGTLVTVS
S.

The HCDR1 of nemolizumab comprises the amino acid sequence GYIMN (SEQ ID NO: 8), the HCDR2 comprises the amino acid sequence LINPYNGGTDYNPQFQD (SEQ ID NO: 9), and the HCDR3 comprises the amino acid sequence DGYDDGPYTLET (SEQ ID NO: 10).

The light chain variable region of nemolizumab comprises the amino acid sequence:

(SEQ ID NO: 11)
DIQMTQSPSSLSASVGDRVTITCQASEDIYSFVAWYQQKP
GKAPKLLIYNAQTEAQGVPSRFSGSGSGTDFTLTISSLQP
EDFATYYCQHHYDSPLTFGGGTKVEIKR.

The LCDR1 of nemolizumab comprises the amino acid sequence QASEDIYSFVA (SEQ ID NO: 12), the LCDR2 comprises the amino acid sequence NAQTEAQ (SEQ ID NO: 13), and the LCDR3 comprises the amino acid sequence QHHYDSPLT (SEQ ID NO: 14).

For the purposes of this disclosure “variant antibodies” or “variant” of nemolizumab may include, but are not limited to: (i) antibodies with heavy chains comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's heavy chain sequence, (ii) antibodies with light chains comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's light chain sequence, (iii) antibodies with variable regions comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's variable region sequences, (iv) antibodies with CDRs comprising at least 55%, at least 65%, at least 70%, at least 75%, at least 80%, at least 85%, at least 90%, at least 95%, at least 97%, at least 98%, at least 99%, or 100% amino acid sequence identity to nemolizumab's CDR sequences, and (v) combinations thereof. For example, suitable variants include immunoglobulins or immunoglobulin-like molecules with the same or substantially similar heavy and light chain amino acid sequences as nemolizumab. Other suitable therapeutic antibodies may bind to the same isoform of IL-31RA as nemolizumab (e.g., IL31-RAv3), optionally the same epitope of IL-31RA, block or neutralize IL-31RA, or combinations thereof. Additional exemplary therapeutic antibodies are described, for example, in WO 2010/064697.

Variants of nemolizumab and suitable therapeutic antibodies may be monoclonal or polyclonal antibodies. Such monoclonal antibodies having IL31-RA-binding and/or neutralizing activity can be obtained, for example, by the following procedure: anti-IL31-RA monoclonal antibodies are prepared by using as an antigen IL31-RA or a fragment thereof that is derived from a mammal such as human or mouse by known methods, and then antibodies having IL31-RA-binding and/or neutralizing activity are selected from the thus obtained anti-IL31-RA monoclonal antibodies. Specifically, a desired antigen or cells expressing the desired antigen are used as a sensitizing antigen for immunization according to conventional immunization methods. Anti-IL31-RA monoclonal antibodies can be prepared by fusing the obtained immune cells with known parental cells using conventional cell fusion methods, and screening them for monoclonal antibody-producing cells (hybridomas) by conventional screening methods. Animals to be immunized include, for example, mammals such as mice, rats, rabbits, sheep, monkeys, goats, donkeys, cows, horses, and pigs. The antigen can be prepared using the known IL31-RA gene sequence according to known methods, for example, by methods using baculovirus (for example, WO 98/46777). Variants of nemolizumab and suitable therapeutic antibodies may also include intrabodies, peptibodies, nanobodies, single domain antibodies, multi-specific antibodies (e.g., bispecific antibodies, diabodies, triabodies, tetrabodies, tandem di-scFV, tandem tri-scFv), darpins, heavy chain monomers, heavy chain dimers, or single-domain antibodies (i.e., a V_{H}H fragment or a “camelid-like” antibody), any of which may be derived from the sequence and/or binding domain of nemolizumab.

Hybridomas can be prepared, for example, according to the method of Milstein et al. (Kohler, G. and Milstein, C., Methods Enzymol. (1981) 73: 3-46). When the immunogenicity of an antigen is low, immunization may be performed after linking the antigen with a macromolecule having immunogenicity, such as albumin. Antigens used to prepare monoclonal antibodies that have a binding and/or neutralizing activity against human IL31-RA are not particularly limited, as long as they enable preparation of antibodies that have a binding and/or neutralizing activity against human IL31-RA. For example, it is known that there are a number of variants of human IL31-RA, and any variant may be used as an immunogen as long as it enables preparation of antibodies that have a binding and/or neutralizing activity against human IL31-RA. Alternatively, under the same condition, a peptide fragment of IL31-RA or a protein in which artificial mutations have been introduced into the natural IL31-RA sequence may be used as an immunogen. Human IL31-RA.3 is one of preferred immunogens in preparing antibodies that have an activity of binding and/or neutralizing IL31-RA in the present disclosure.

The IL31-RA-binding activity of therapeutic antibodies can be determined by methods known to those skilled in the art. Methods for determining the antigen-binding activity of an antibody include, for example, ELISA (enzyme-linked immunosorbent assay), EIA (enzyme immunoassay), RIA (radioimmunoassay), and fluorescent antibody method. For example, when enzyme immunoassay is used, antibody-containing samples, such as purified antibodies and culture supernatants of antibody-producing cells, are added to antigen-coated plates. A secondary antibody labeled with an enzyme, such as alkaline phosphatase, is added and the plates are incubated. After washing, an enzyme substrate, such as p-nitrophenyl phosphate, is added, and the absorbance is measured to evaluate the antigen-binding activity. The binding and/or neutralizing activity of a therapeutic antibody against IL31-RA can be measured, for example, by observing the effect of suppressing the growth of the IL-31-dependent cell line. For example, the activity of a purified mouse IL-31 antibody can be assayed by assessing the IL-31-dependent growth of Ba/F3 cells transfected with mouse IL-31 receptor α and mouse OSMR genes.

Any of the anti-IL31RA antibodies (i.e. “therapeutic antibodies”) disclosed herein, including nemolizumab and fragments or variants thereof, can be used for treating and/or preventing PN and achieving the disclosed therapeutic endpoints. Optimal doses and routes of administration may vary.

### IV. PHARMACEUTICAL COMPOSITIONS

Provided herein are pharmaceutical compositions for use in the treatment or prevention of prurigo nodularis (PN), including skin lesions or nodules or pruritus caused by PN. The pharmaceutical compositions comprise an anti-IL31RA antibody (i.e. “therapeutic antibody”), such as nemolizumab or a fragment or variant thereof, as an active ingredient.

The phrase “comprise(s) nemolizumab or a fragment or variant thereof as an active ingredient” means comprising nemolizumab or a fragment or variant thereof as at least one of the active ingredients, and does not limit the proportion of the antibody. In addition, the therapeutic agents for PN in the present disclosure may also comprise, in combination with nemolizumab or a fragment or variant thereof, other ingredients that enhance the treatment or prevention of PN. For example, the composition may comprise one or more topical corticosteroid creams or injections, ointments with menthol or phenol to cool and soothe itchy skin, capsaicin cream, oral corticoseroids, selective serotonin reuptake inhibitors (SSRIs), and oral antihistamines.

Pharmaceutical compositions of nemolizumab or a fragment or variant thereof of the present disclosure can be prepared as formulations according to standard methods (see, for example, Remington's Pharmaceutical Science, Mark Publishing Company, Easton, USA). The pharmaceutical compositions generally comprise a carrier and/or additive in addition to the antibody. For example, in some embodiments, the pharmaceutical composition comprises one or more surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, corrigents, light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. In some embodiments, the pharmaceutical composition comprises one or more other low-molecular-weight polypeptides, proteins such as serum albumin, gelatin, and immunoglobulin, and amino acids such as glycine, glutamine, asparagine, arginine, and lysine.

When nemolizumab or a fragment or variant thereof may be prepared as an aqueous solution for injection, in which nemolizumab or a fragment or variant thereof may be dissolved in an isotonic solution containing, for example, physiological saline, dextrose, or other excipients or tonifiers (i.e., tonicity agents). The tonifier may include, for example, D-sorbitol, D-mannose, D-mannitol, and sodium chloride. In addition, appropriate solubilizing agents, for example, alcohols (for example, ethanol), polyalcohols (for example, propylene glycols and PEGs), and non-ionic detergents (polysorbate 80 and HCO-50) may be used concomitantly.

In some embodiments, nemolizumab or a fragment or variant thereof may be encapsulated in microcapsules (microcapsules made of hydroxymethylcellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition” &, Oslo Ed. (1980)). Moreover, methods for making sustained-release drugs are known, and these can be applied for nemolizumab or a fragment or variant thereof (Langer et al., J. Biomed. Mater. Res. (1981) 15, 167-277; Langer, Chem. Tech. (1982) 12, 98-105; U.S. Pat. No. 3,773,919; European Patent Application (EP) No. 58,481; Sidman et al., Biopolymers (1983) 22, 547-56; EP 133,988).

The pharmaceutical compositions of the present disclosure can be administered either orally or parenterally, but are preferably administered parenterally. Specifically, the pharmaceutical compositions are administered to patients by injection or percutaneous administration. Injections include, for example, intravenous injections, intramuscular injections, and subcutaneous injections, for systemic or local administration. The pharmaceutical compositions may be given to sites where inflammation and/or itching is to be suppressed, or areas surrounding the sites by local infusion or intramuscular or subcutaneous injection. In some embodiments, the pharmaceutical compositions are administered at the site of one or more skin excoriations, lesions, or nodules, or proximal to the site of one or more skin excoriations, lesions, or nodules.

The administration methods can be properly selected according to the patient's age, weight, and condition. The single-administration dose can be selected, for example, from within the range of 0.0001 to 100 mg of the antibody (e.g., nemolizumab or a fragment or variant thereof) per kg body weight. Alternatively, for example, when the antibody is administered to human patients, the dose of the antibody can be selected from within the range of 0.001 to 1,000 mg/kg body weight. In some embodiments, the composition is formulated to administer a dose containing, for example, about 0.01 to 50 mg/kg, about 0.01 mg/kg to about 0.1 mg/kg, about 0.05 mg/kg to 0.15 mg/kg, about 0.1 mg/kg to about 0.6 mg/kg, about 0.1 mg/kg to about 1 mg/kg, about 0.25 mg/kg to about 0.75 mg/kg, about 0.4 mg/kg to about 0.8 mg/kg, about 0.4 mg/kg to about 1.8 mg/kg, about 0.5 to about 2.5 mg/kg, about 0.8 mg/kg to about 2.2 mg/kg, about 1 mg/kg to about 2.5 mg/kg, about 1 mg/kg to about 3.5 mg/kg, about 1 mg/kg to about 5 mg/kg, about 2 mg/kg to about 4 mg/kg, about 2.5 mg/kg to about 10 mg/kg, about 5 mg/kg to about 10 mg/kg, about 10 mg/kg to about 20 mg/kg, about 10 mg/kg to about 40 mg/kg, about 20 mg/kg to about 50 mg/kg, about 25 mg/kg to about 75 mg/kg, about 50 mg/kg to about 100 mg/kg, or about 100 mg/kg to about 500 mg/kg, or about 100 mg/kg to about 1000 mg/kg body weight of nemolizumab or a fragment or variant thereof. In preferred embodiments, the dose ranges from about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg. In some embodiments, the dose is about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In particular embodiments, the effective amount of nemolizumab or a fragment or variant thereof is about 0.1 mg/kg, about 0.5 mg/kg, about 1 mg/kg, about 1.5 mg/kg, about 2 mg/kg, or about 2.5 mg/kg. In a preferred embodiment, the dose is about 0.5 mg/kg.

The present disclosure provides a pharmaceutical composition for use in the treatment or prevention of prurigo nodularis (PN) in a subject, comprising an anti-IL31RA antibody (e.g., nemolizumab or a fragment or variant thereof), as an active ingredient, wherein the subject differentially expresses at least one gene selected from the genes disclosed in Table 1 compared to a reference level of expression for the at least one gene.

The present disclosure provides a pharmaceutical composition for use in the treatment or prevention of prurigo nodularis (PN) in a subject, comprising an anti-IL31RA antibody (e.g., nemolizumab or a fragment or variant thereof), as an active ingredient, wherein the subject differentially expresses at least one gene selected from the genes disclosed in Table 1 compared to a reference level of expression for the at least one gene, and wherein the subject is diagnosed as having prurigo nodularis (PN), by detecting in a sample obtained from a subject suspected of having PN the expression level of at least one, at least two, at least three, at least four, or at least five of the differentially expressed genes (DEGs) in Table 1, and comparing the expression level of the DEGs to a reference level, wherein the reference level is the corresponding level of gene expression for each DEG in a sample from an individual that does not have PN.

Any of the pharmaceutical compositions disclosed herein, including nemolizumab and fragments or variants thereof, can be used for treating and/or preventing PN and achieving the disclosed therapeutic endpoints. Optimal doses and routes of administration may vary.

### V. METHODS OF TREATMENT/PREVENTION AND COMPOSITIONS FOR TREATING OR PREVENTING PN

The present disclosure provides methods of treating or preventing pruritus in a subject having prurigo nodularis (PN), the method comprising, consisting of, or consisting essentially of administering an anti-IL-31RA antibody (i.e., a “therapeutic antibody”), such as nemolizumab or a fragment or variant thereof to the subject. The disclosed methods may be performed to achieve specific therapeutic endpoints, which are discussed in more detail below. Also disclosed herein are uses of an anti-IL-31RA antibody (i.e., a “therapeutic antibody”), such as nemolizumab or a fragment or variant thereof to the subject for treating or preventing PN and/or achieving the disclosed therapeutic endpoints. Also disclosed herein are anti-IL-31RA antibodies (i.e., a “therapeutic antibodies”), such as nemolizumab or a fragment or variant thereof to the subject for use in treating or preventing PN and/or achieving the disclosed therapeutic endpoints. Additionally, particular subgroups of subjects with PN may be especially suitable for treatment according to the disclosed methods and uses (e.g., patients presenting with any of the DEGs disclosed in Table 1).

The present disclosure is the first to report a transcriptomic signature for PN that is able to not only identify and positively diagnose PN, but also identify subjects with PN that will likely response to treatment with an anti-IL-31RA antibody (e.g., nemolizumab) and track the response of the treatment with an anti-IL-31RA antibody (e.g., nemolizumab).

The present disclosure is also the first to report a plasma proteome signature for PN subjects that were successfully treated with an anti-IL-31RA antibody, such as nemolizumab. This “responder signature” may be used not only as a marker of positive clinical endpoints, but also identify subjects with PN that will likely response to treatment with an anti-IL-31RA antibody (e.g., nemolizumab) and track the response of the treatment with an anti-IL-31RA antibody (e.g., nemolizumab).

A. Subject Being Treated

A subject being treated for PN according to the disclosed methods and uses may exemplify one or more of the underlying gene expression patterns that are disclosed herein. In particular, a subject with PN that is to be treated according to the disclosed methods and uses may differentially express up to or at least 5,943 genes (known as differentially expressed genes or DEGs), which are shown in FIG. 1A and Table 1 below. This differential gene expression may be observed in the skin of the subject and, in particular, in a skin sample comprising or consisting of a nodule or lesion. Of these DEG, 2,060 may be increased (i.e., overexpressed) and 3,874 may be decreased (i.e., under expressed). Genes that may be increased the most include:
KRT6C, which may be increased at least 100-fold, at least 150 fold, at least 200-fold, at least 250-fold, at least 300-fold, at least 350-fold, at least 400-fold, at least 450-fold, at least 500-fold, at least 550-fold, or 588-fold compared to the expression level in a sample (e.g., a skin sample) from an individual that does not have PN;
DEFB4A, which may be increased at least 25-fold, at least 50-fold, at least 75-fold, at least 100-fold, at least 125-fold, or at least 150 fold compared to the expression level in a sample (e.g., a skin sample) from an individual that does not have PN; and
KRT16, which may be increased at least 10-fold, at least 20-fold, at least 30-fold, at least 40-fold, at least 50-fold, at least 60-fold, at least 70-fold, at least 80-fold, or at least 90 fold compared to the expression level in a sample (e.g., a skin sample) from an individual that does not have PN.

Decreased genes include:
LCE5A, which may be decreased at least 2-fold, at least 3-fold, at least 4-fold, at least 5-fold, at least 6-fold, at least 7-fold, at least 8-fold, at least 9-fold, at least 10-fold, or at least 11 fold compared to the expression level in a sample (e.g., a skin sample) from an individual that does not have PN; and
AQP7, which may be decreased at least 2-fold, at least 3-fold, at least 4-fold, at least 5-fold, at least 6-fold, at least 7-fold, or at least 7.9 fold compared to the expression level in a sample (e.g., a skin sample) from an individual that does not have PN.

Genes that encode cytokines may also be overexpressed in subjects suffering from PN that can be treated or prevented according to the disclosed methods and uses. For the cytokine genes, the most prominent up-regulated genes are IL-36 family members and IL-20 family members. These upregulated or overexpressed genes may include:
IL36A (e.g., about 6.8-fold, FDR=1.8×10⁻⁴);
IL36G (e.g., about 8.4-fold, FDR=3.9×10⁻²⁵);
IL19 (e.g., about 5.1-fold, FDR=7.4×10⁻⁴);
IL20 (e.g., about 3.5-fold, FDR=1.7×10⁻³);
IL22 (e.g., about 2.7-fold, FDR=2.9×10⁻²);
IL24 (e.g., about 5.8-fold, FDR=3.8×10⁻¹⁰); and
IL26 (e.g., about 4.9-fold, FDR=3.3×10⁻³).

Each of these IL-36 and IL-20 family member cytokine genes may be overexpressed by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, or at least about 8-5-fold compared to the expression level in a sample (e.g., a skin sample) from an individual that does not have PN.

Other factors that may be upregulated or overexpressed in a sample (e.g., a skin sample) obtained from a subject with PN that is to be treated may include ILIA (e.g., about 4.7-fold, FDR=1.0×10⁻¹²), IL1B (e.g., about 4.1-fold, FDR=3.7×10⁻⁶, and IL4R (e.g., about 2.6-fold (FDR=6.3×10¹⁹). Table 1 at the end of the examples section of the specification provides a more exhaustive list of the DEGs.

Certain gene ontology (GO) categories also may be evident in the skin of subjects with PN that are treated according to the disclosed methods and uses. These GO categories are:
“cornified envelope” (FDR=1.5×10⁻¹²),
“epidermal cell differentiation” (FDR=6.4×10⁻¹⁰),
“keratinization” (FDR=1.6×10⁻¹²),
“peptidase regulator activity” (FDR=1.1×10⁻⁴),
“interleukin-4 and 13 signaling” (FDR=6.8×10⁻⁷),
“interferon alpha beta signaling” and “response to interferon gamma” (FDR=4.1×10⁻⁷, and FDR=4.1×10⁻⁶, respectively),
“IL23 pathway” (FDR=2×10⁻⁵), and
“mitotic metaphase and anaphase” (FDR=3.8×10⁻¹⁰).

These categories reflect the hyperproliferative nature of PN, which was discovered to be associated with altered epidermal differentiation and inflammatory components. In some embodiments, a subject with PN that is treated according to the disclosed methods and uses may overexpress the proliferative marker Ki67 (MKI67), the cell cycle gene CDKN1A, and/or inflammatory networks involving IL-1 and IL-36.

A subject with PN that is treated according to the disclosed methods or uses may additionally or alternatively present with one or more of the co-expression modules or clusters shown in FIG. 1D and Table 3 at the end of the examples section of the specification. Distinct functions are defined for these co-expressing gene modules, as noted herein.

The present disclosure also provides specific cell-type signatures for non-lesional and lesional PN skin that can be present in subjects with PN that are treated according to the disclosed methods and uses. For example, a subject with PN may possess the transcriptomic signature associated with epithelial cells and keratinocytes was observed in FIG. 2A and/or may possess a Th2 associated signature, as described herein. Other inflammatory signatures, such as macrophages, may also be prominent in PN lesions and the skin of subjects with PN.

In general, the disclosed methods and uses can treat or prevent PN in subjects that suffer from mild, moderate, or severe pruritus. In some embodiments, the PN may be categorized as moderate to severe. In some embodiments, the PN may be categorized as moderate, while in other embodiments, the PN may be categorized as severe. In some embodiments, pruritus can be scored as none, mild, moderate, or severe. “None,” “mild,” “moderate,” and “severe” are terms of art in describing the presence, extent, and/or intensity of excoriations. Those of skill in the art know the metes and bounds of these terms. For example, pruritus can be characterized according to one or more of the following methods known by those skilled in the art. The intensity can be quickly measured with monodimensional scales that are routinely used in clinical care. See Pereira et al., Allergology International (2017) 66:3-78. Additionally or alternatively, patients can be asked to rate their itch intensity from 0 (“no itch”) to 10 (“worst imaginable itch”) with the numerical rating scale (NRS). Another monodimensional scale, the visual analogue scale (VAS), provides patients with the opportunity to indicate the intensity of their itch by marking on a 10 cm long, ruler-shaped scale. Both endpoints are marked with a number corresponding to the intensity, with 0 representing “no itch” and 10 the “worst imaginable itch.” Scores below 3.0 VAS/NRS points are generally associated with mild itch, whereas scores higher than 6.9 illustrate severe itch. Scores above 9.0 represent a very severe itch. The verbal rating scale (VRS) is a further monodimensional scale that allows patients to describe their itch intensity by means of gradually rising adjectives (0—no itch, 4—worst imaginable itch). The NRS, VAS and VRS have been validated in large-scale studies consisting of chronic pruritus patients with pruritic dermatoses or pruritus of various origins. These instruments have high reproducibility and there was a high correlation between scales 6, 7, 8. Chronic pruritus can greatly reduce patient quality of life. For this reason, Dermatology Life Quality Index (DLQI) is widely used and has been validated. DLQI scores range from 0 to 30, with higher scores indicating a lower quality of life. Investigators' Global Assessment (IGA) scores range from 0 (clear) to 5 (very severe disease) and are presented as a percentage of patients in the indicated population. In the present study, IGA scores range from 0 to 4.

A subject being treated for PN according to the disclosed methods and uses may exemplify one or more of the disclosed plasma protein signature alterations that are disclosed herein. In particular, a subject with PN that is to be treated according to the disclosed methods and uses may exhibit (a) a decrease in migration and cell movement of leukocytes; (b) an inhibition of STAT3 and STAT5b pathways; (c) a downregulation in IL-6 and VEGF pathways; (d) a decrease in TGFB1 pathway, or (e) a combination thereof. The foregoing decreases or inhibitions may be observed at a specified time point after the commencement of treatment, such as 2 weeks, 4 weeks, 6 weeks, 8 weeks, 10 weeks, or 12 weeks. The decreases or inhibitions may be determined relative to (i) a control sample obtained from an individual or individuals without PN or (ii) a biological sample obtained from the subject prior to administration of the anti-IL-31RA antibody. Additionally or alternatively, the subject may exhibit an upregulation in a neuronal ontology terms including CREB signaling in neurons, Synaptogenesis signaling pathway, Cell death of neuroglia and Apoptosis of neuroglia and a combination thereof following administration of the anti-IL-31RA antibody.

A subject with PN that is suitable for treatment according to the disclosed methods and uses may exhibit high levels of leukocyte migration or leukocyte cell movement relative to an individual or population without PN or in the subject prior to commencing treatment with an anti-IL-31RA antibody, such as nemolizumab. Additionally or alternatively, a suitable subject may exhibit STAT3 pathway activity or expression that is comparatively high relative to an individual or population without PN or in the subject prior to commencing treatment with an anti-IL-31RA antibody, such as nemolizumab. Additionally or alternatively, a suitable subject may exhibit TGFB1 pathway activity or expression that is comparatively high relative to an individual or population without PN or in the subject prior to commencing treatment with an anti-IL-31RA antibody, such as nemolizumab. Additionally or alternatively, a suitable subject may exhibit amounts of circulating cytokine signature that are comparatively high in a subject with PN relative to an individual or population without PN or in the subject prior to commencing treatment with an anti-IL-31RA antibody, such as nemolizumab. Such cytokine pathways can include, but are not limited to IL-6, and VEGF pathways.

Prior to treatment with an anti-IL-31Ra antibody, such as nemolizumab, the amount of the disclosed protein markers in the plasma of a subject with PN may be at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold higher than a baseline level. The baseline level may be determined relative to (i) a control sample obtained from an individual or individuals (i.e., a population) without PN or (ii) a biological sample obtained from the subject prior to administration of the anti-IL-31RA antibody.

In some embodiments, the subject has been diagnosed of PN for at least about 6 months. In particular embodiments, the subject has at least about 20 nodules on his/her body with a bilateral distribution. In particular embodiments, the subject has prurigo lesions on upper limbs, with or without lesions on the trunk or lower limbs. In some embodiments, the pruritus has been assigned a score of at least 7 on the Numerical Rating Scale (NRS). In some embodiments, the mean of the worst daily intensity of the NRS score is at least 7 over the previous 3 days. In some embodiments, the mean of the worst daily intensity of the NRS score is at least 7 over the previous week.

In some embodiments, the subject does not have atopic dermatitis (AD). In some embodiments, the subject does not have chronic pruritus resulting from a condition other than PN, such as scabies, insect bite, lichen simplex chronicus, psoriasis, acne, folliculitis, habitual picking, lymphomatoid papulosis, chronic actinic dermatitis, dermatitis herpetiformis, sporotrichosis, or bullous disease. In some embodiments, the subject does not have neuropathic or psychogenic pruritus, such as notalgia paresthetica, brachioradial pruritus, dilutional parasitosis, or pathomimia.

B. Therapeutic Endpoints for Treatment

Based on the data provided herein, it is believed that IL-31 signaling is upstream of IL-17 and IL-4 signaling, at least in subjects with PN, as treatment with the anti-IL-31RA antibody nemolizumab was able to alter the transcriptome expression signature of IL-17 and IL-4. Accordingly, the disclosure provides methods of normalizing differentially expressed genes (DEGs) in subjects with PN, comprising administering to a subject with PN an anti-IL-31RA antibody, such as nemolizumab or a fragment or variant thereof. As shown in the Examples section below, treatment or prevention with nemolizumab leads to normalization of a number of PN associated DEGs for both increased (e.g., about 969 genes), and decreased genes (e.g., about 1,268 genes). See FIG. 4A. In some embodiments, about 5, about 10, about 15, about 20, about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, or about 2500 DEGs may be normalized in a subject with PN after treatment with an anti-IL-31RA antibody, such as nemolizumab or a fragment or variant thereof. In some embodiments, at least 5, at least 10, at least 15, at least 20, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, or at least 2500 DEGs and up to 2500, 3000, 3500, 4000, 4500, 5000, 5500, or about 6000 DEGs may be normalized in a subject with PN after treatment with an anti-IL-31RA antibody, such as nemolizumab or a fragment or variant thereof. In some embodiments, treatment or prevention may comprise administration of the anti-IL-31RA antibody (e.g., nemolizumab) once a week, once every 2 weeks, once every 3 week, once every 4 weeks, once every 5 weeks, once every 6 week, once every 7 week, or once every 8 weeks. Administration about once every 4 weeks may be preferred. Administration may be perform via injection, such as subcutaneous injection. The timeframe for determining/comparing the normalization of the DEGs may be 4 week, 5 weeks, 6 weeks, 7 weeks, 8 week, 9 weeks, 10 weeks, 11 weeks, or 12 weeks or more. For example, a baseline (i.e., at or before treatment commences) a sample (e.g., a skin sample) may be obtained from the subject to detect the level of expression of some or all of the putative DEGs disclosed in Table 1 (e.g., KRT6C, DEFB4A, KRT16, LCE5A, AQP7, IL-36 family members, IL-20 family members, etc.), and another sample can be taken and assessed at 4 week, 5 weeks, 6 weeks, 7 weeks, 8 week, 9 weeks, 10 weeks, 11 weeks, or 12 weeks or more weeks after treatment has commenced in order to determine whether the expression level of the DEGs has changed and in more in line (i.e., “normalized”) with reference expression levels of the DEGs that are associated with normal, healthy skin (e.g., expression levels obtained from a skin sample from a subject without PN). Such methods can be used to track treatment and assess whether a subject is responsive to the treatment with the anti-IL-31RA antibody (e.g., nemolizumab).

GO categories enriched amongst DEGs may also be decreased or altered as a result of treatment with an anti-IL-31RA antibody (e.g., nemolizumab). For example, after 12 weeks of treatment, the following GO categories were observed to decrease: “cell cycle” (FDR=5.6×10⁻¹⁴), “keratinocyte differentiation” (FDR=1.8×10⁻⁴), and “interleukin-4 and 13 signaling” (FDR=1.5×10⁻²). Indeed, the data provided in the Examples section demonstrates that nemolizumab treatment (and treatment with other anti-IL-31RA antibodies) normalizes epidermal hyperproliferation, normalizes differentiation, and decreases inflammatory responses, particularly related to Th2 responses. Accordingly, the present disclosure provides methods of normalizing epidermal hyperproliferation, normalizing epidermal differentiation, and/or decreasing inflammatory responses in the skin, comprising administering to a subject with PN an anti-IL-31RA antibody (e.g., nemolizumab). In some embodiments, the inflammatory response may be a Th2 response. In some embodiments, administration of the anti-IL-31RA antibody (e.g., nemolizumab) may be once a week, once every 2 weeks, once every 3 week, once every 4 weeks, once every 5 weeks, once every 6 week, once every 7 week, or once every 8 weeks. Administration about once every 4 weeks may be preferred. Administration may be perform via injection, such as subcutaneous injection.

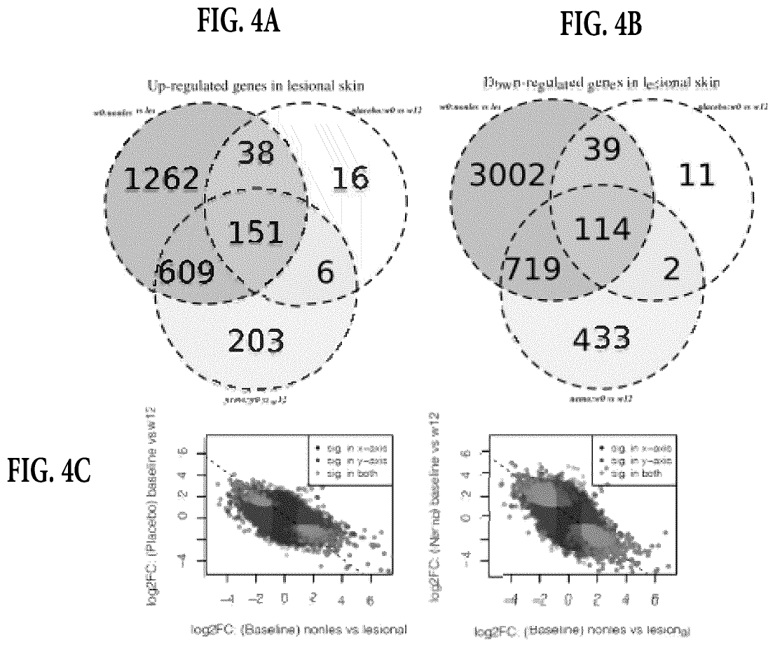

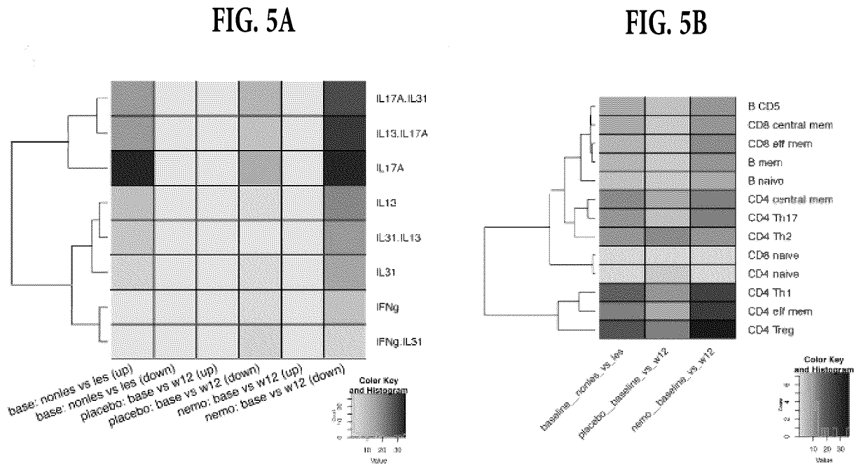

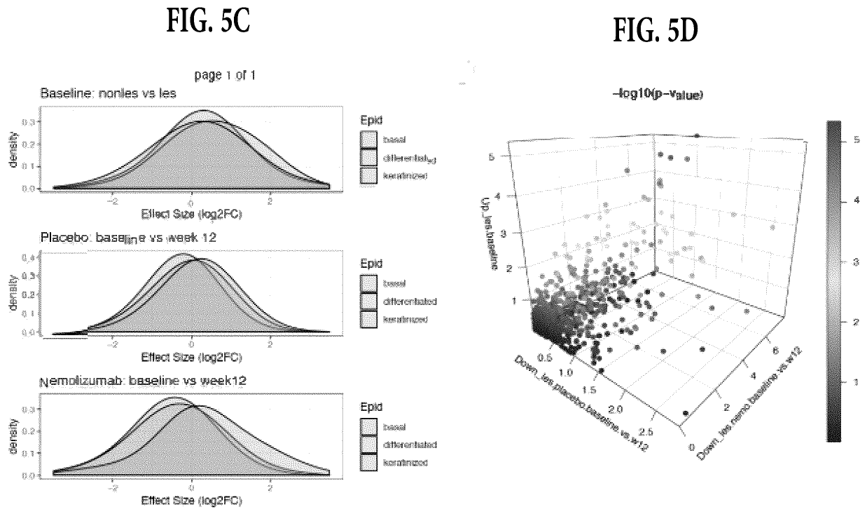

With respect to the disclosed methods of decreasing inflammatory responses in the skin of a subject with PN, in some embodiments, the cytokine response signatures generated in human epidermal rafts of the subject's skin may be altered. For example, IL17A mRNA expression was not significantly different in non-lesional vs lesional skin, nor was it different after a 12 week treatment with nemolizumab. A consistent decrease in IL-31 responses, either solitary, or in combination with other inflammatory cytokines, including the Th2 cytokine IL-13 or IL-17A can be seen in FIG. 5A, thus providing evidence of blockade of the IL-31 pathway by nemolizumab. However, IL-17A response genes are shown herein to be enriched in PN skin and are downregulated by nemolizumab. This suggests that while IL-17A itself is not a dominant cytokine in PN, it is down-stream of IL-31 signaling. Indeed, a consistent decrease in IL-31 responses, either solitary or in combination with other inflammatory cytokines (including the Th2 cytokine IL-13, or IL-17A), was observed with treatment with nemolizumab (FIG. 5A), and more robust decreases were observed in Th1, Th17, and Th2 markers (FIG. 5B). Accordingly, IL-31 signaling is likely upstream of IL-17 and IL-4 signaling.

In some embodiment, a basal keratinocyte (KRT14+) signature may be elevated in the lesional skin of PN, and this signature may be restored to normal (i.e., non-PN) when treated according to the disclosed methods and uses. In some embodiments, a spinous layer (KRT10+) signature may be elevated in the PN lesional skin, and this signature may be restored to normal (i.e., non-PN) when treated according to the disclosed methods and uses (FIG. 5C).

In some embodiments, transcription factor binding site (TFBS) that are enriched among genes up-regulated in baseline lesional skin are more likely to be enriched among the nemolizumab down-regulated genes following treatment with an anti-IL-31RA antibody, such as nemolizumab (e.g., by week 12 post-treatment, as shown in FIG. 5D). In some embodiments, the transcription factor that is down regulated by treatment can be EGR4 (which is a member of the EGF family of zinc finger transcription factors), STAT3, and/or KLF16.

The present disclosure additionally provides plasma protein markers that have been identified in subjects that were successfully treated with nemolizumab, and therefore the resulting alteration in biomarkers can be considered a “responder signature” for establishing success, as well as a means of tracking responsiveness. Thus, disclosed herein are methods of treating or preventing prurigo nodularis (PN) in a subject, comprising administering to a subject with PN an anti-IL-31RA antibody, wherein treatment with the anti-IL-31RA antibody results in: (a) a decrease in leukocyte migration and cell movement, (b) a decrease in IL-6 or a decrease in IL-6 pathway signaling, (c) a decrease in VEGF or a decrease in VEGF pathway signaling, (d) a decrease in STAT3 or a decrease in STAT3 pathway signaling, (e) a decrease in STAT5b or a decrease in STAT5b pathway signaling, (f) a decrease in TGFB1 or a decrease in TGFB1 pathway signaling, or (g) a combination thereof. In some embodiments, the subject may also exhibit an increase in the disclosed neuronal ontologies. Additionally, disclosed here are methods of altering an immune response in a subject with PN, comprising administering to a subject with PN an anti-IL-31RA antibody, wherein treatment with the anti-IL-31RA antibody results in: (a) a decrease in leukocyte migration and cell movement, (b) a decrease in IL-6 or a decrease in IL-6 pathway signaling, (c) a decrease in VEGF or a decrease in VEGF pathway signaling, (d) a decrease in STAT3 or a decrease in STAT3 pathway signaling, (e) a decrease in STAT5b or a decrease in STAT5b pathway signaling, (f) a decrease in TGFB1 or a decrease in TGFB1 pathway signaling, or (g) a combination thereof. In some embodiments, the subject may also exhibit an increase in the disclosed neuronal ontologies. The disclosed plasma protein markers are detectable by, for example, mass spectrometry and other methods of protein assessment (e.g., ELISA, Western blot, etc.).

In particular, when a subject with PN is treated with an anti-IL-31RA antibody, such as nemolizumab, the immune cells (e.g., leukocytes) may experience a decrease migration or cell movement or both.

STAT3 pathway activity, may decrease in a subject with PN that is treated with an anti-IL-31RA antibody, such as nemolizumab, relative to an individual or population without PN or to the subject prior to commencing treatment with an anti-IL-31RA antibody, such as nemolizumab.

The amount of cytokine activity may also be comparatively high in a subject with PN relative to an individual or population without PN or in the subject prior to commencing treatment with an anti-IL-31RA antibody, such as nemolizumab. Such cytokine pathways can include, but are not limited to IL-6 and VEGF pathways. In a PN subject that receives treatment with an anti-IL-31RA antibody, such as nemolizumab, the treatment may result in a decrease in IL-6 or VEGF signatures or both relative to a baseline level. The baseline level may be determined relative to (i) a control sample obtained from an individual or individuals (i.e., a population) without PN or (ii) a biological sample obtained from the subject prior to administration of the anti-IL-31RA antibody.

TGFB1 pathway activity may decrease in a subject with PN that is treated with an anti-IL-31RA antibody, such as nemolizumab, relative to an individual or population without PN or the subject prior to commencing treatment with an anti-IL-31RA antibody, such as nemolizumab.

For the purposes of the disclosed plasma protein markers, the amount of the disclosed protein markers in the plasma of a subject with PN may be at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold higher than a baseline level. The baseline level may be determined relative to (i) a control sample obtained from an individual or individuals (i.e., a population) without PN or (ii) a biological sample obtained from the subject prior to administration of the anti-IL-31RA antibody. Similarly, after a subject with PN is treated with an anti-IL-31RA antibody, such as nemolizumab (e.g., 2 weeks, 4 weeks, 6 weeks, 8 weeks, 10 weeks, or 12 weeks after administration of the antibody), the amount of the disclosed plasma protein markers in the subject may decrease at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold relative to a baseline level. The baseline level may be determined relative to (i) a control sample obtained from an individual or individuals (i.e., a population) without PN or (ii) a biological sample obtained from the subject prior to administration of the anti-IL-31RA antibody.

Additionally, neuronal ontologies (e.g., CREB signaling in neurons, Synaptogenesis signaling pathway, Cell death of neuroglia and Apoptosis of neuroglia) may be upregulated in subject with PN, and subsequently downregulated in nemolizumab responders subjects.

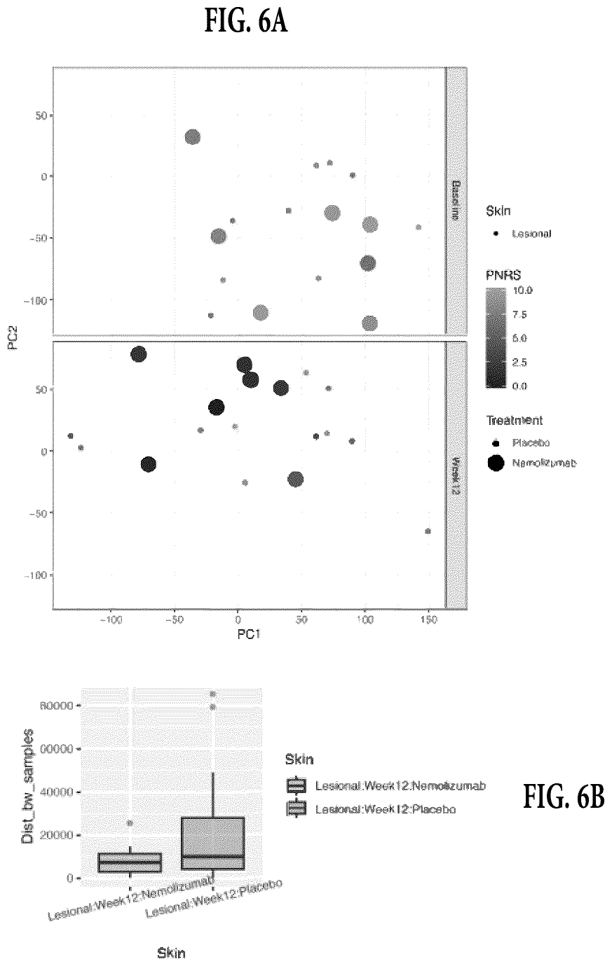

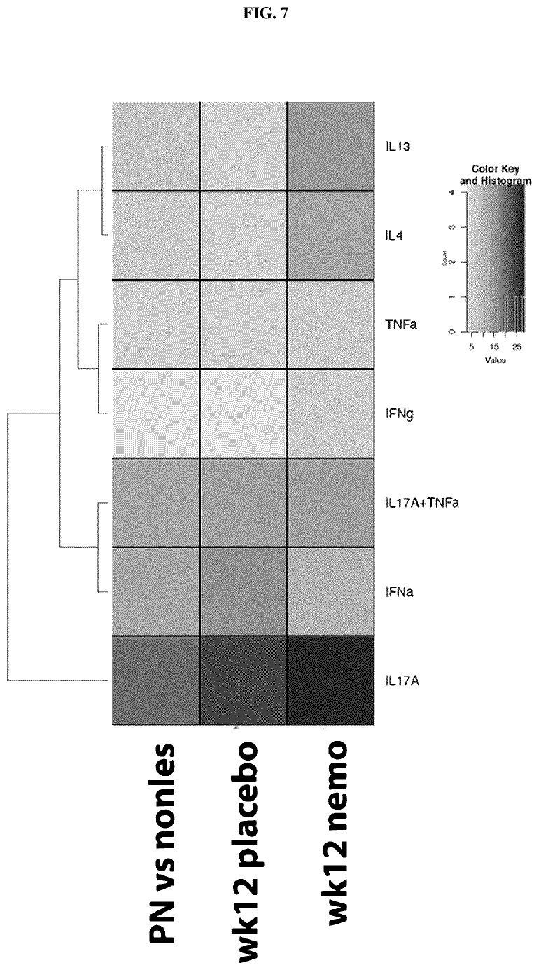

In some embodiments of the disclosed methods and uses, treatment or prevention with an anti-IL-31RA antibody, such as nemolizumab or a fragment or variant thereof, leads to decrease in pruritus scoring. The decrease in scoring may be measured by, for example, the peak pruritus numeric rating scale (PP-NRS). See, e.g., FIG. 6A. Indeed, the data provided herein show that all subject with PN that were treated with nemolizumab showed improvement in pruritus scoring. In some embodiments, the measured distance between the principle component 1 and principle component 2 (i.e., PC1/PC2 components) may be less than that of a placebo group that was not treated with nemolizumab or another anti-IL-31RA antibody. More specifically, at Week 12 (i.e., 12 weeks after commencing treatment) the proportion of patients achieving 4-point reduction of weekly average of the PP NRS was significantly higher in the nemolizumab group compared to placebo (52.9% versus 8.3%, p<0.001). At Week 12 the proportion of subjects achieving IGA Success (Defined as IGA 0 [Clear] or 1 [Almost Clear]) was significantly higher in the nemolizumab group compared to placebo (20.6% versus 2.8%, p=0.02).

C. Fibroblast Response to Treatment

As described herein, the present disclosure was the first to establish that PN is an inflammatory and fibrotic disease, and that by antagonizing IL-31 signaling (e.g., via treatment with nemolizumab), it is possible to exerts an anti-fibrotic effect via inhibiting critical signaling pathways. In particular, compared to healthy skin, lesional PN fibroblasts take on a pro-fibrotic and pro-inflammatory state, which can result in differential expression of genes in lesional PN fibroblasts that are involved in activation of inflammatory (TNF, IL1B, IL6) and pro-fibrotic (TGFβ) signaling pathways. Indeed, the PN fibroblast plays a central player in the intra-cellular cross-talk, and treatment with an anti-IL-31RA antibody, such as nemolizumab or a fragment or variant thereof, restores neural dysregulation and reduces inflammation and fibrosis.

For example, the present disclosure provides a method of treating or preventing prurigo nodularis (PN) in a subject, comprising administering to a subject with PN an anti-IL-31RA antibody, wherein the subject shows activation of tumor necrosis factor (TNF) signaling in a lesional skin cell compared to a reference level of activation of the TNF signaling.

Similarly, the present disclosure provides a method of normalizing activation of a tumor necrosis factor (TNF) expression in a subject with PN, comprising administering to a subject with PN an anti-IL-31RA antibody, wherein the subject shows activation of tumor necrosis factor (TNF) signaling in a lesional skin cell compared to a reference level of expression of the TNF gene and wherein administration of the anti-IL-31RA antibody normalizes the expression level of the TNF gene. For the purposes of such a method, normalization can be determined about 4 weeks, about 8 weeks, or about 12 weeks after administration of the anti-IL-31RA antibody.

For the purposes of these methods, differential expression and pathway activation can be determined by RT-qPCR, RT-PCR, RNA-seq, Northern blotting, Serial Analysis of Gene Expression (SAGE), or DNA or RNA microarrays. Additionally or alternatively, differential expression and pathway activation may also be determined at protein level by Western blotting, ELISA, surface plasmon resonance, or mass spectrometry.

TNF activation in the lesional skin cell, such as a fibroblast, may be higher prior to treatment compared to the reference level of expression. The reference level may be level of activation is a level of activation of the TNF signaling in a skin cell (e.g., a fibroblast) of a person that does not have PN. Additionally or alternatively, the reference level is the level activation of the TNF gene in a non-lesional skin cell of the subject.

The present disclosure also provides a method of decreasing inflammation in the skin of a subject with prurigo nodularis (PN), comprising administering to a subject with PN an anti-IL-31RA antibody, thereby decreasing inflammation involving tumor necrosis factor (TNF) signaling in the skin. In such methods, TNF signaling in skin of the subject is overexpressed relative to a reference level of activation of the TNF signaling, optionally, wherein the TNF signaling is activated in a fibroblast. The reference level may be the level of activation of the TNF signaling in a skin cell (e.g., a fibroblast) of a person that does not have PN. Alternatively, the reference level may be the level of activation of the TNF signaling in a non-lesional skin cell of the subject.

In some embodiments of these methods, the inflammation further involves IL-1 pathway signaling, IL-6 pathway signaling, TGFβ pathway signaling, or any combination thereof.

The present disclosure also provides a method of treating or preventing prurigo nodularis (PN) in a subject, comprising administering to a subject with PN an anti-IL-31RA antibody, wherein treatment with the anti-IL-31RA antibody results in a decrease in tumor necrosis factor (TNF) pathway activation. In some embodiments, the decrease in TNF pathway activation occurs in lesional skin of the subject. In some embodiments, the decrease in TNF pathway activation occurs in a fibroblast of the subject.

As the present disclosure indicates, there can often be overlapping signaling involved in the pathogenesis of PN. Accordingly, for the purposes of the disclosed methods the treatment further results is:
(a) a decrease in migration of leukocytes or cell movement of leukocytes;
(b) an inhibition of a STAT3 pathway;
(c) an inhibition of a STAT5b pathway;
(d) a downregulation of IL-1 or an IL-1 pathway;
(e) a downregulation of IL-6 or an IL-6 pathway;
(f) a downregulation of VEGF or a VEGF pathway;
(g) a decrease in TGFB1 pathway activation, or
(h) a combination thereof.

In some embodiments, the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is determined relative to (i) a control sample obtained from an individual or individuals without PN or (ii) a biological sample obtained from the subject prior to administration of the anti-IL-3 IRA antibody.

In some embodiments, (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is assessed after about 4 weeks, about 8 weeks, or about 12 weeks after the administration of the anti-IL-31RA antibody.

In some embodiments, (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is determined by mass spectrometry performed on one or more biological sample(s) obtained from the subject. In some embodiments, the one or more biological sample(s) is a plasma sample or a skin sample.

In some embodiments, the subject exhibits at least two of, at least three of, at least four of, at least five of, at least six, or all seven of (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; and (g) the decrease in TGFB1 pathway activation.

D. Doses and Dosing Regimen for the Disclosed Methods and Uses

An effective amount of an anti-IL-31RA antibody, such as nemolizumab or a fragment or variant thereof, is an amount sufficient to effect beneficial or desired results such as alleviating at least one or more symptom of PN. An effective amount as used herein would also include an amount sufficient to delay or prevent the development pruritus, alter the course of a PN symptom, or reverse a symptom of PN. Thus, it is not possible to specify the exact “effective amount.” However, for any given case, an appropriate “effective amount” can be determined by one of ordinary skill in the art using only routine experimentation.

An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment and prevention dosages generally may be titrated to optimize safety and efficacy. The dosage can be determined by a physician and adjusted, as necessary, to suit observed effects of the treatment. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

Dosage regimens for treating or preventing PN may comprise flat dosing (i.e., administering the same dose repeatedly at pre-determined intervals) or comprise a loading dose (i.e., administrating an initial dose that is higher or different than subsequent, serial doses). For the purposes of either type of dosing regimen an effective dose may be administered topically, parenterally, subcutaneously, subdermally, intradermally, or intramuscularly. In preferred embodiments, administration comprises subcutaneous injection.

In some embodiments, a loading dose and the subsequent serial doses may be administered via the same route (e.g., subcutaneously), while in some embodiments, a loading dose and the subsequent serial doses may be administered via different routes (e.g., parenterally and subcutaneously, respectively). In some embodiments, the loading dose may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or higher. In some embodiments, the loading dose may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, or higher. In some embodiments, the loading dose may be about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In some embodiments, the loading dose may be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 15 mg/kg, 25 mg/kg, 50 mg/kg, 75 mg/kg, 100 mg/kg, 500 mg/kg, or 1,000 mg/kg. In some embodiments, the loading dose is administered as a single injection. In some embodiments, the loading dose is administered as multiple injections, which may be administered at the same time or spaced apart at defined intervals.

The subsequent serial doses of a loading dose regimen are generally lower than the loading dose. For examples, in some embodiments, the dosing regimen may comprise a loading dose of 60 mg and a serial dose of 30 mg, which may be administered a defined interval of, for example, every 4 weeks. In some embodiments, the serial dose of a dosing regimen may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or higher. In some embodiments, the serial dose may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, or higher. In some embodiments, the serial dose may be about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In some embodiments, the serial dose may be 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 15 mg/kg, 25 mg/kg, 50 mg/kg, 75 mg/kg, 100 mg/kg, 500 mg/kg, or 1,000 mg/kg.

For the purposes of a loading dose regimen, the first serial dose may be administered 1 day, 2 days, 3 days, 4 days, 5 days, 6 days, 7 days, 8 days, 9 days, 10 days, 11 days, 12 days, 13 days, 1 week, 2 weeks, 3 weeks, 4 weeks, 5 weeks, 6 weeks, 7 weeks, 8 weeks, 9 weeks, or 10 weeks after the initial loading dose. In some embodiments, the first serial dose is administered 4 weeks after the initial loading dose. In some embodiments, the subsequent serial doses are administered once every 1 day, 2 days, 3 days, 4 days, 5 days, 6 days, 7 days, 8 days, 9 days, 10 days, 11 days, 12 days, 13 days, 1 week, 2 weeks, 3 weeks, 4 weeks, 5 weeks, 6 weeks, 7 weeks, 8 weeks, 9 weeks, or 10 weeks. In some embodiments, the serial doses are spaced 4 weeks apart (i.e., nemolizumab or a fragment or variant thereof is administered once every 4 weeks).

In some embodiments, the dose of nemolizumab or a fragment or variant thereof administered to the subject can be within the range of 0.001 to 1,000 mg/kg body weight of the subject. In some embodiments, the dose ranges from about 0.01 to 50 mg/kg, about 0.01 mg/kg to about 0.1 mg/kg, about 0.05 mg/kg to 0.15 mg/kg, about 0.1 mg/kg to about 0.6 mg/kg, about 0.1 mg/kg to about 1 mg/kg, about 0.25 mg/kg to about 0.75 mg/kg, about 0.4 mg/kg to about 0.8 mg/kg, about 0.4 mg/kg to about 1.8 mg/kg, about 0.5 to about 2.5 mg/kg, about 0.8 mg/kg to about 2.2 mg/kg, about 1 mg/kg to about 2.5 mg/kg, about 1 mg/kg to about 3.5 mg/kg, about 1 mg/kg to about 5 mg/kg, about 2 mg/kg to about 4 mg/kg, about 2.5 mg/kg to about 10 mg/kg, about 5 mg/kg to about 10 mg/kg, about 10 mg/kg to about 20 mg/kg, about 10 mg/kg to about 40 mg/kg, about 20 mg/kg to about 50 mg/kg, about 25 mg/kg to about 75 mg/kg, about 50 mg/kg to about 100 mg/kg, or about 100 mg/kg to about 500 mg/kg, or about 100 mg/kg to about 1000 mg/kg body weight of nemolizumab or a fragment or variant thereof. In preferred embodiments, the dose ranges from about 0.01 mg/kg to about 0.1 mg/kg, about 0.1 mg/kg to about 0.5 mg/kg, about 0.5 mg/kg to about 1.5 mg/kg, about 1.5 mg/kg to about 2.5 mg/kg, or about 2.5 mg/kg to about 10 mg/kg. In some embodiments, the dose is about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 25 mg/kg, about 50 mg/kg, about 75 mg/kg, about 100 mg/kg, about 500 mg/kg, or about 1,000 mg/kg. In particular embodiments, the dose of nemolizumab or a fragment or variant thereof is about 0.1 mg/kg, about 0.5 mg/kg, about 1 mg/kg, about 1.5 mg/kg, about 2 mg/kg, or about 2.5 mg/kg. In a preferred embodiment, the dose is about 0.5 mg/kg.

In some embodiments, the dose of nemolizumab or a fragment or variant thereof administered to the subject is within the range of 1 to 100 mg, 25 to 75 mg, 30 to 60 mg, 40 to 80 mg, 20 to 80 mg, 1 to 25 mg, 1 to 50 mg, 10 to 90 mg, 15 to 85 mg, or ranges there between. In some embodiments, the dose may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or higher. In some embodiments, the dose may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, or higher.

In some embodiments of the disclosed methods and uses, a loading dose of about 60 mg of nemolizumab or a fragment or variant thereof may be administered to a subject with PN, followed by subsequent serial doses of nemolizumab of a fragment or variant thereof at about 30 mg once every 4 weeks. In some embodiments of the disclosed methods and uses a first dose of about 60 mg of nemolizumab or a fragment or variant thereof may be administered to a subject with PN, followed by subsequent serial doses of nemolizumab of a fragment or variant thereof at about 60 mg once every 4 weeks (i.e., the dose remains constant or is a “flat” dosing regimen).

In some embodiments of the disclosed methods and uses a first dose of about 30 mg of nemolizumab or a fragment or variant thereof may be administered to a subject with PN, followed by subsequent serial doses of nemolizumab of a fragment or variant thereof at about 30 mg once every 4 weeks

In some embodiments of the disclosed methods and uses, nemolizumab or a fragment or variant thereof is administered by a topical or parenteral route. In some embodiments, nemolizumab or a fragment or variant thereof is administered subcutaneously. In some embodiments, the dose is administered subcutaneously at or proximal to a site of one or more nodules, lesions, or excoriations.

In some embodiments of the disclosed methods and uses, nemolizumab or a fragment or variant thereof is administered daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, and/or as needed based on the appearance of symptoms of PN. In preferred embodiments, nemolizumab or a fragment or variant thereof is administered every four weeks or every eight weeks.

In some embodiments of the disclosed methods and uses, the duration of treatment or prevention is about one day, about one week, about two weeks, about three weeks, about four weeks, about five weeks, about six weeks, about seven weeks, about eight weeks, about nine weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 24 weeks, about 30 weeks, about 36 weeks, about 40 weeks, about 48 weeks, about 50 weeks, about one year, about two years, about three years, about four years, about five years, or as needed based on the appearance of symptoms of PN. In preferred embodiments, duration of treatment or prevention is about 12 weeks to about 24 weeks, about 12 to about 36 weeks, about 12 to about 48 weeks, or about 24 to about 36 weeks.

The present disclosure provides uses of nemolizumab or a fragment or variant thereof in the manufacture of a medicament for the treatment or prevention of PN, for normalizing differentially expressed genes (DEGs) in subjects with PN, for normalizing epidermal hyperproliferation, for normalizing epidermal differentiation, and/or for decreasing inflammatory responses in the skin. All of the disclosed doses, dosing regimens, routes of administrations, biomarkers, and therapeutic endpoints are applicable to these uses as well.

The following examples are given to illustrate the present disclosure. It should be understood that the invention is not to be limited to the specific conditions or details described in these examples.

### EXAMPLES

### Example 1—Treatment of Prurigo Nodularis with Nemolizumab

Methods

Patient Cohort

The current study was performed in patients with moderate to severe PN. Briefly, 70 patients were randomized 1:1 to placebo (36 patients) or nemolizumab (34 patients), the latter at a dose of 0.5 mg/kg body weight administered at baseline, week 4 and week 8. Peak pruritus score on the numerical rating scale (PP-NRS) was recorded: severity of pruritus on the numerical rating scale ranged from 0 (no itch) to 10 (worst itch imaginable) and the peak pruritus was estimated using the worst scores every 24 hours in a 7-day period, with the highest score recorded as peak score. Primary outcome of the study was percent change from baseline in the PP-NRS at week 4.

Reconstructed Human Epidermis (RHE) Model

Three-dimensional RHE models were generated. Briefly, RHE cultures were generated using Normal Human Dermal Fibroblasts (NHDF) and Normal Human Epidermal Keratinocytes (NHEK). RHE cultures used were full thickness, with a dermis and an epidermis, and composed of autologous Fibroblasts and Keratinocytes. They were scaffold-free (no exogenous matrix) allowing self-assembly of the different layers of the skin by the cells and avoiding some potential inhibitors contained in collagen matrix for the RNA extractions. The RHE were cultivated in 12 well plates with insert (1.2 cm²) for 42 days to obtain a minimum of two layers of dermis and six layers of epidermis. RHE cultures from 6 different donors were left unstimulated or stimulated with 7 different cytokines or cytokine combinations (3 replicates per donor per condition): IL-31, IL-13, IL-17A, IFNg, IL-31+IL-13, IL-31+IL-17A, IL-31+ IFNg and IL-13+IL-17A. Concentrations for each of the cytokines used were: IL-31 (500 ng/mL), IL-13 (100 ng/mL), IL-17A (200 ng/mL), IFNg (50 ng/mL). RHE cultures were lysed 72 hours after stimulation, and RNA extraction was performed using MagMAX mirVana Total RNA isolation from Tissue Kit (ThermoFisher Scientific). RNA was purified and concentrated using RNA Clean & Concentrator-5 kit (Zymo Research), according to the protocol, and RNA concentrations ranged from 3 to 380 ng/μL. Total RNA was quantified using the QuantiFluor One RNA kit (Promega) on the GloMax-Multi+ Detection System (Promega). Library preparation was performed using the Illumina® Stranded mRNA Prep Ligation kit (Illumina), according to manufacturer's recommendations.

Skin Biopsy Processing and RNA Isolation

Skin biopsies were collected from 16 placebo and 15 nemolizumab-treated subjects.

Samples included lesional and non lesional biopsies at baseline as well as lesional samples after 12 weeks of treatment (placebo or nemolizumab). RNA was extracted from skin biopsies using Tripure Isolation Reagent (Sigma-Aldrich) according to manufacturer's instructions. From these RNA samples, a DNase treatment was applied using the RNase-Free DNase kit (Cat No. 79254, Qiagen), followed by the RNeasy® MinElute® Cleanup Kit (Cat No. 74204, Qiagen).

Total RNA was quantified using the QuantiFluor One RNA kit (Promega). RNA concentration obtained ranging from 4 to 20 ng/μl. Total RNA was qualified using Fragment Analyzer 5300 (Agilent) with the Agilent HS RNA Fragment kit (Agilent). The RNA Quality Number (RQN) obtained ranging from 1 to 6. Library preparation was performed using the SMARTer Stranded Total RNA-Seq Kit V2—Pico Input Mammalian kit (TaKaRa).

RNA-Seq

Libraries were quantified using the QuantiFluor One dsDNA kit (Promega) and library analysis was performed using Fragment Analyzer 5300 (Agilent) with the Agilent HS NGS Fragment kit (Agilent). Following size selection with AMPure XP beads, shotgun libraries were sequenced using NextSeq (Illumina) on an Illumina NextSeq500 sequencer, in 2×75 bp (High Output Kit v2, 150 cycles).

RNA-Seq Dataprocessing

Following adapter trimming, sequence reads for 83 unique samples were aligned to human genome (GRCh37) using STAR. Gene (GENCODE v29) expression levels were then quantified with HTSeq using reads uniquely mapped to one genome location. Two RNA-seq samples were identified as outliers and 81 samples were used in subsequent analysis. Only genes with an average of at least one read per sample were retained. DESeq2 was used for expression normalization, and the negative binomial distribution was used to model the expression level for differential expression analysis. For non-lesional vs lesional and the baseline vs week 12 comparison, individual effect was included as covariate; for the placebo vs nemolizumab comparison, age and gender were controlled. False Discovery Rate (FDR)≤5% and |log₂ Fold Change|>=1 were used as criteria to declare significant differentially expressed genes (DEGs).

Cytokine, Cell Signature, and Functional Inference Analysis

The most significant DEGs were compared against the transcripts induced by cytokines in keratinocytes (defined by FDR<=10% and 1.5-Fold Change (FC)). The top 1,000 most significant DEGs from the PN data were used for fair comparison. For the comparison against epidermal compartments gene signature, scRNA-seq was conducted on the epidermal layer of skin biopsy, and identified the top 50 marker genes for the basal, differentiated, and keratinized layers, respectively. The effect size (in log₂ FC) was then investigated for each marker gene in each differential expression comparison. For transcription analysis, promoter region was defined as 5,000 base pairs upstream of the transcription start site, and the MEME suite was used to compute the enrichment statistics for the transcription factor binging.

Weighted Gene Correlation Network Analysis (WGCNA)

Gene that were expressed in at least 20% of the samples were used in the dataset for the WGCNA. The “softPower” parameter was picked as the smallest value that achieve at least r2>=0.75. Spearman correlation was used to compute the correlation, and minimum module size was set as 100. Upon the module merging, a height cut of 0.2 was used.

Results

Prurigo Nodularis is Characterized by Abnormal Keratinocyte Differentiation and Immune Activation

After quality control, RNA-sequencing data analysis of biopsy tissue from patients with prurigo nodularis (PN) was performed. There were 31 PN patients with the transcriptomic data in both lesional and non-lesional skin samples. Using false-discovery rate (FDR) of ≤10% and |log₂|>1 as criteria, 5,943 differentially expressed genes (DEGs) were identified when comparing the uninvolved and lesional skin at baseline, of which 2,060 genes were increased and 3,874 were decreased (FIG. 1A). Genes that showed the most robust increase included KRT6C (588-fold, FDR=8.2×10⁻⁸⁰), DEFB4A (150-fold, FDR=1.1×10⁻¹²), and KRT16 (90-fold, FDR=1.9×10⁻⁵²). Decreased genes included LCE5A (11-fold decrease, FDR=8.1×10⁻¹⁸) and AQP7 (7.9-fold decrease, FDR=2.6×10⁻¹⁷). For the cytokines, the most prominent up-regulated genes included the IL36-family members: IL36A (6.8-fold, FDR=1.8×10⁻⁴) and IL36G (8.4-fold, FDR=3.9×10⁻²⁵); IL-20 family members: IL19 (5.1-fold, FDR=7.4×10⁻⁴), IL20 (3.5-fold, FDR=1.7×10⁻³), IL22 (2.7-fold, FDR=2.9×10⁻²), IL24 (5.8-fold, FDR=3.8×10⁻¹⁰), and IL26 (4.9-fold, FDR=3.3×10⁻³). Other factors included ILIA (4.7-fold, FDR=1.0×10⁻¹²), and IL1B (4.1-fold, FDR=3.7×10⁻⁶). The Th2 cytokines IL4 and IL13 did not reach significance but the IL4R was increased by 2.6-fold (FDR=6.3×10¹⁹) (see Table 1 at the end of the examples section of the specification).

Functional enrichment analysis was then performed on the DEGs to define the biological processes associated with PN skin. The most prominent gene ontology (GO) terms included: “cornified envelope” (FDR=1.5×10⁻¹²), “epidermal cell differentiation” (FDR=6.4×10⁻¹⁰), “keratinization” (FDR=1.6×10⁻¹²), “peptidase regulator activity” (FDR=1.1×10⁻⁴), “interleukin-4 and 13 signaling” (FDR=6.8×10⁻⁷), “interferon alpha beta signaling” and “response to interferon gamma” (FDR=4.1×10⁻⁷, and FDR=4.1×10⁻⁶, respectively), “IL23 pathway” (FDR=2×10⁻⁵), and “mitotic metaphase and anaphase” (FDR=3.8×10⁻¹⁰) (FIG. 1B)(see also Table 2 at the end of the examples section of the specification). These reflect the hyperproliferative nature of PN, associated with altered epidermal differentiation and the inflammatory components; by focusing on the key expression modules in the lesional skin transcriptome, the proliferative marker Ki67 (MKI67), the cell cycle gene CDKN1A, and inflammatory networks involving IL-1 and IL-36 were revealed (FIG. 1C).

To gain a better understanding of the disease regulatory networks involved in PN skin weighted gene co-expression network analysis (WGCNA) was performed. 20 co-expression modules were identified in non-lesional skin and 10 clusters in lesional PN skin (FIG. 1D) (see Table 3 at the end of the examples section of the specification). This analysis allowed the inventors to assign distinct functions for these co-expressing gene modules, especially for the co-expression modules in PN lesional skin, with the most prominent involved immunological processes (module #8) including “immune-response” (FDR=1.8×10⁻⁴⁷), “defense response” (FDR=1.2×10⁻³⁹); cell proliferation (module #6) including “cell cycle” (FDR=2.9×10⁻⁹⁴), “DNA metabolic process” (FDR=8.7×10⁻⁶⁷); and epidermal processes (module #5), such as “epidermis development” (FDR 3.5×10⁻¹⁰), “keratinization” (FDR=1.7×10⁻⁶). (FIG. 1E). Other notable findings were changes in “extracellular matrix” (FDR=1.16×10⁻⁵⁹; module #2) and included genes such as MMP14, MMP16, COL1A1, COL1A2, and COL3A1, which were modestly elevated (FC≥1.4; FDR≤6×10⁻²) in the lesional skin, consistent with the association of PN with skin fibrosis.

Transcriptomic Changes in PN Lesion are Enriched for Keratinocyte and T-Cell Signatures

An in silico approach (xCell) was used to infer specific cell-type signatures for each non-lesional and lesional PN skin sample. Enrichment for transcriptomic signatures associated with epithelial cells and keratinocytes was observed (p<0.001 and p<0.0001, respectively) (FIG. 2A). There was also increased prominence of Th2 associated signature (p<0.0001), consistent with the enriched GO categories for IL-4/IL-13 (FIG. 1). Other inflammatory signatures, such as macrophages (p<0.01), were more variable (FIG. 2A). To address the relationship of PN with other hyperproliferative skin diseases that also have strong inflammatory signatures, the PN transcriptome was compared against that of atopic dermatitis (AD) and psoriasis. In a 3-way comparison there was a large number of genes that were shared between all three diseases for both up-regulated and down-regulated genes (FIG. 2B). The correlation of the effect sizes in the lesional skin was more pronounced between PN and psoriasis (Spearman correlation p=0.64) than between PN and AD (ρ=0.55). Genes that are commonly up-regulated in both psoriasis and PN include those participating in cytokine activity (CCL3, CXCL10, IFNG, IL12B, IL19, IL1B, IL20, etc.) and keratinization (KRT16, KRT17, LCE3A, LCE3E, etc.). See Table 4 at the end of the examples section of the specification.

Transcriptomic Changes in PN Skin with the IL31 Receptor Inhibitor Nemolizumab

Clinical results in patients with PN showed that nemolizumab resulted in a higher percentage of improvement of pruritus and skin lesions with overall good safety profile. At Week 12 the proportion of patients achieving 4-point reduction of weekly average of the PP-NRS was significantly higher in the nemolizumab group compared to placebo (52.9% versus 8.3%, p<0.001). At Week 12 the proportion of subjects achieving IGA Success (Defined as IGA 0 [Clear] or 1 [Almost Clear]) was significantly higher in the nemolizumab group compared to placebo (20.6% versus 2.8%, p=0.02).

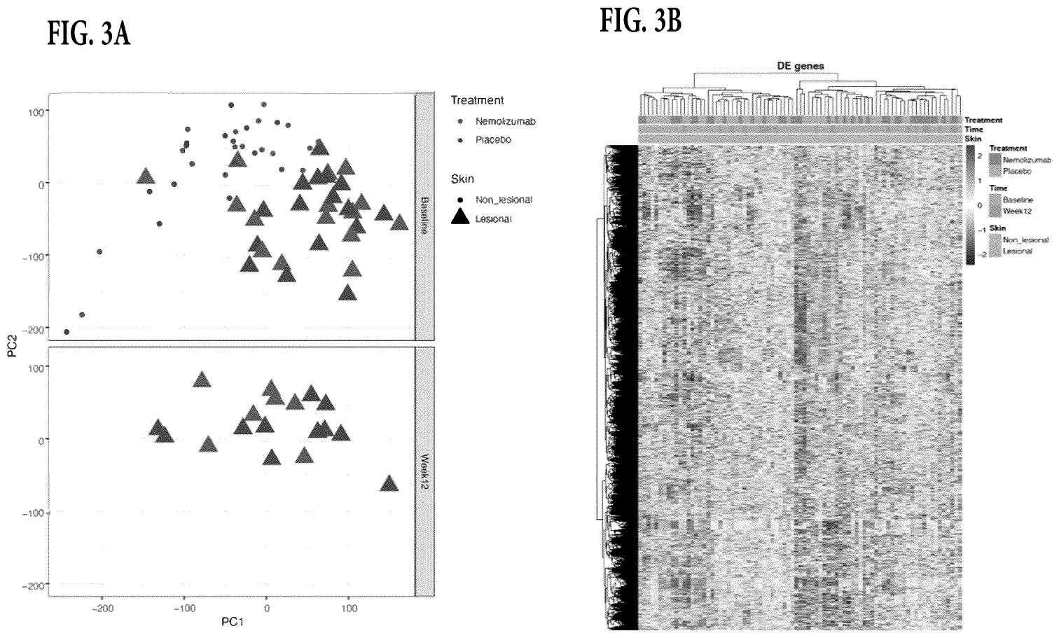

To address the therapeutic effect of the IL-31 receptor (IL-31R) inhibitor nemolizumab, RNA-seq data was performed from PN biopsies prior to and after 12-weeks of treatment, and used placebo controlled in a double-blinded study. At baseline, there were 16 and 15 individuals in the placebo and nemolizumab groups, respectively, and at week 12 lesional samples were obtained from 18 of the patients (11 placebo and 7 nemolizumab). Using principal component analysis (PCA) admixing of samples was observed from patients with PN at baseline between the two treatments groups (placebo vs nemolizumab). After 12 weeks of treatment, there was a trend towards grouping of the nemolizumab, but not the placebo cohort (FIG. 3A). This was accompanied by sample clustering using genes that were identified to be differentially expressed in the non-lesional vs lesional skin comparison, with 6 out of the 7 nemolizumab samples (86%) in week 12 grouping with the baseline non-lesional skin samples, versus 57% (4 out of 7) of the placebo group (FIG. 3B). Notably, nemolizumab treatment led to normalization of greater number of PN associated DEGs compared to placebo for both genes upregulated in PN lesional skin (969 nemolizumab vs. 211 placebo) and genes downregulated in PN lesional skin (1,268 genes nemolizumab vs. 166 for placebo) (FIG. 4A). This was also reflected in the correlation between placebo and nemolizumab treated DEGs with much greater overlap between nemolizumab treated group and PN, when compared to placebo vs. PN for both increased and decreased DEGs (FIG. 4B). GO categories enriched amongst DEGs decreased only in the nemolizumab treated group by week 12 included “cell cycle” (FDR=5.6×10⁻¹⁴), “keratinocyte differentiation” (FDR=1.8×10⁻⁴), and “interleukin-4 and 13 signaling” (FDR=1.5×10⁻²), whereas none of these GO categories were found in the placebo controlled group (see Table 2 at the end of the examples section of the specification). These data demonstrate that nemolizumab treatment normalized both epidermal hyperproliferation and differentiation, in addition to decreasing inflammatory responses, particularly related to Th2 responses.

Nemolizumab Response is Accompanied by Decreased IL-31 Th2 Responses in PN Skin

To address the effect of nemolizumab treatment on inflammatory responses in PN skin, nemolizumab and placebo treatment response was interrogated against cytokine response signatures generated in RHE cultures as well as human epidermal rafts. A consistent decrease in IL-31 responses was observed, either solitary, or in combination with other inflammatory cytokines, including the Th2 cytokine IL-13 or IL-17A (FIG. 5A), providing clear evidence of blockade of the IL-31 pathway by nemolizumab. Notably, IL-17A response genes were enriched in PN skin, likely corresponding to the specific downstream immunological cascade overlapping between psoriasis and PN (Table 4). The IL17A mRNA expression was itself not significantly different in the non-lesional vs lesional skin, or in the nemolizumab treatment by week 12 (see Table 1 at the end of the examples section of the specification), suggesting that while IL-17A is not a dominant cytokine in PN IL-17A signatures are downstream of IL-31 signaling. In terms of cellular transcriptomic changes, more robust decrease was observed in the nemolizumab group including Th1 and Th17 (FIG. 5B).

To determine the tissue compartment that has the largest contribution to cellular response to anti-IL-31R blockade, the transcriptomic data from placebo and nemolizumab groups was compared against gene signatures for epidermal compartments obtained from single-cell RNA-seq data. The results indicate the basal keratinocyte (KRT14+) signature was elevated in the lesional skin of PN, while this latter was restored in similar degree in both the placebo and the treatment groups, whereas the induction of the spinous layer (KRT10+) signature in the PN lesional skin was only restored by the treatment but not the placebo group (FIG. 5C). Transcription factor binding site (TFBS) analyses was then performed to further understand the transcriptional regulators of the transcriptomic changes in PN and following placebo or nemolizumab treatment. The results demonstrated that binding sites that are enriched among genes up-regulated in the baseline lesional skin are more likely to be enriched among the nemolizumab down-regulated genes by week 12 (FIG. 5D). The most significantly regulated transcription factors included EGR4 (p=4.5×10⁻⁶ and p=1.2×10⁻⁸ for enrichment in the promoters of up-regulated genes and for nemolizumab down-regulated genes, respectively), a member of the EGF family of zinc finger transcription factors; STAT3 (p=2.2×10⁻⁴ and p=2.5×10⁻⁵, respectively); and KLF16 (p=4.5×10⁻⁵ and p=2×10⁻⁵, respectively) (see Table 5 at the end of the examples section of the specification).

Nemolizumab Leads to Decrease in Pruritus Scoring

Peak pruritus numeric rating scale (PP-NRS), PP-NRS was associated with the transcriptome data (FIG. 6A). While PP-NRS was similar at baseline in both groups, there was consistent decrease in pruritus in the nemolizumab group only, whereas there was wide range of responses in the placebo group. Also, whereas all patients in the nemolizumab group showed improvement, only a subset of patients exhibited significant changes in the placebo group. Notably, the measured distance between the principle component 1 and principle component 2 (PC1/PC2) components for the nemolizumab treated group was much less than that of the placebo group (FIG. 6B), consistent with a therapeutic response.

### Discussion

The data presented here are the first to provide a comprehensive view of the global transcriptomic changes in PN skin, and to reveal novel and important insights into the mechanism of action and efficacy of the anti-IL-31 receptor inhibitor nemolizumab. Notably, these data reflect on the transcriptomic level many of the hallmark histologic changes observed in PN, including epidermal alteration, inflammatory response (FIGS. 2A-2C), fibrosis, and pruritus (FIGS. 6A-6B), and how these normalize with nemolizumab treatment.

Many of changes in gene expression in PN skin are related to abnormal keratinocyte proliferation and differentiation (FIGS. 1A-1E). These epidermal alterations accounted for most of the overlap of PN with both AD and psoriasis (FIGS. 2A-2C; Table 4), both of which are also characterized by marked epidermal hyperplasia and altered epidermal differentiation. Notably, these changes demonstrated significant improvement in the nemolizumab treated group by week 12 of treatment, but not in the placebo group (Table 2). Furthermore, consistent with the therapeutic effect of nemolizumab, the greatest effect of normalization was observed on the differentiated layer of the epidermis (FIG. 5C), which may be reflective of decreased keratinocyte proliferation and restoration of normal epidermal differentiation.

Consistent with PN being a disease process driven by inflammation, immune responses such as Th2 (IL-4/IL-13) response and type I and type II IFN responses were prominent (FIGS. 1A-1E). Th2 responses closely correlate with itch in diseases such as atopic dermatitis, a common predisposing disease to PN development. Interestingly, anti-IL-31 receptor inhibition significantly decreased not only IL-31 responses in keratinocytes in PN skin (FIG. 5A) but also led to decrease in Th2 responses, as well as decreased Th17 (FIG. 5B), corresponding to decreased IL-13 and IL-17 responses in keratinocytes (FIG. 5A). The enriched IL-17 responses in PN skin, as well as contribution from IL-36, likely account for the greater overlap of PN skin with plaque psoriasis, in contrast to AD (FIGS. 2B and 2C). No significant changes were observed in IL17A mRNA expression in the PN data, suggesting that IL-17A is not a dominant cytokine in PN. In addition, the changes in both Th2 and Th17 responses with nemolizumab treatment, and to a lesser extent changes in type II IFN responses, suggest that these cytokines act downstream of IL-31 in PN.

Fibrosis is a feature of PN, most prominent in the papillary dermis, and less commonly in the reticular dermis. Fibrosis in PN is characterized by deposition of vertically oriented collagen fibrils. Modules of genes were found that are involved in extracellular matrix biology to be enriched in PN skin (Table 3), involving both collagen 1 and collagen 3 genes. COL1A1, COL1A2, and COL1A3 mRNA were increased in PN skin at baseline (1.7-, 1.44-, and 1.52-fold respectively, see Table 1) but did not show significant changes with nemolizumab treatment at week 12.

This data also demonstrates how nemolizumab driven transcriptomic changes in PN correlate with improvement in pruritus. Chronic pruritus is a debilitating symptom of PN and has a profound impact on quality of life. The etiology of pruritus in PN still remains unclear but possible factors include the Th2 cytokines, IL-4 and IL-13, major pruritogens in atopic dermatitis, versus changes in cutaneous innervation, with decreased density of intraepidermal nerve fibers being shown to be reduced in both lesional and non-lesional skin. The data from the nemolizumab treatment is consistent with both of these scenarios contributing to itch. Thus, nemolizumab treatment leads to suppression of Th2 and IL-4/IL-13 responses in PN skin and also leads to decrease in expression of factors such as KLF16, which has been shown to inhibit neurite growth. In addition, nerve growth factor (NGF), which was confirmed to increase in PN skin, is also normalized with nemolizumab treatment at week 12 to a larger extent than in the placebo group (Table 1). No changes were seen for the expression of CGRP or substance P (TAC1). These data are highly suggestive of a broad effect of nemolizumab on pruritus and may account for the long duration of pruritus improvement seen beyond the last dose of nemolizumab (>2 months).

In summary, PN is a debilitating and a difficult condition to treat, and no therapies are currently approved for its treatment. This work in-depth characterization of the transcriptomic changes in PN skin and demonstrate the broad mechanisms of action of nemolizumab, an anti-IL-31 receptor inhibitor. These data demonstrate the broad therapeutic effect of anti-IL-31 receptor inhibition with nemolizumab on multiple aspects of PN pathogenesis, including epidermal differentiation, inflammatory responses, pruritus and extracellular remodeling, and confirms the upstream role of IL-31 in PN pathogenesis.

### Example 2—Plasma Proteomic Analysis of Nemolizumab Treated Moderate-to-Severe Prurigo Nodularis Patients

This example details a randomized, double-blind, phase 2 trial of the anti-human IL31Ra blocking antibody nemolizumab at a dose of 0.5 mg per kilogram of body weight, administered subcutaneously at baseline, week 4 and week 8, as compared with placebo, in patients with moderate to severe prurigo nodularis. The goal of this study was to characterize the effect of nemolizumab on the entire plasma proteome by using mass spectrometry.

Materials and Methods

Patient Dataset

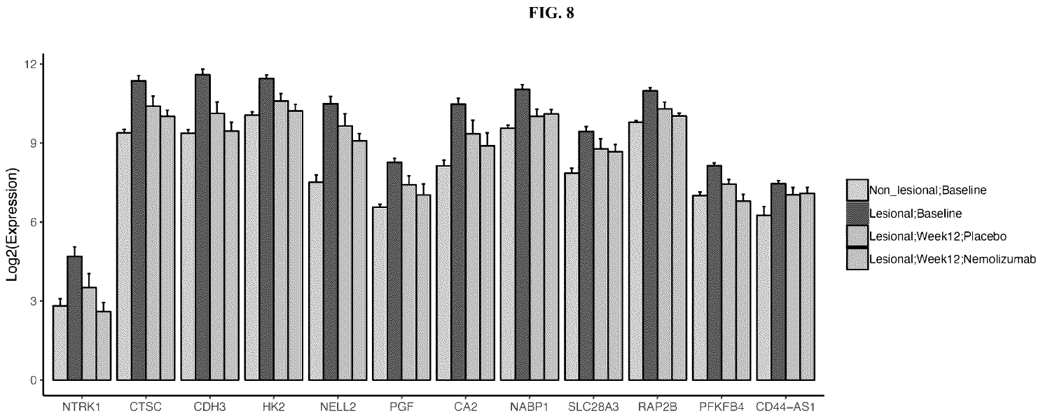

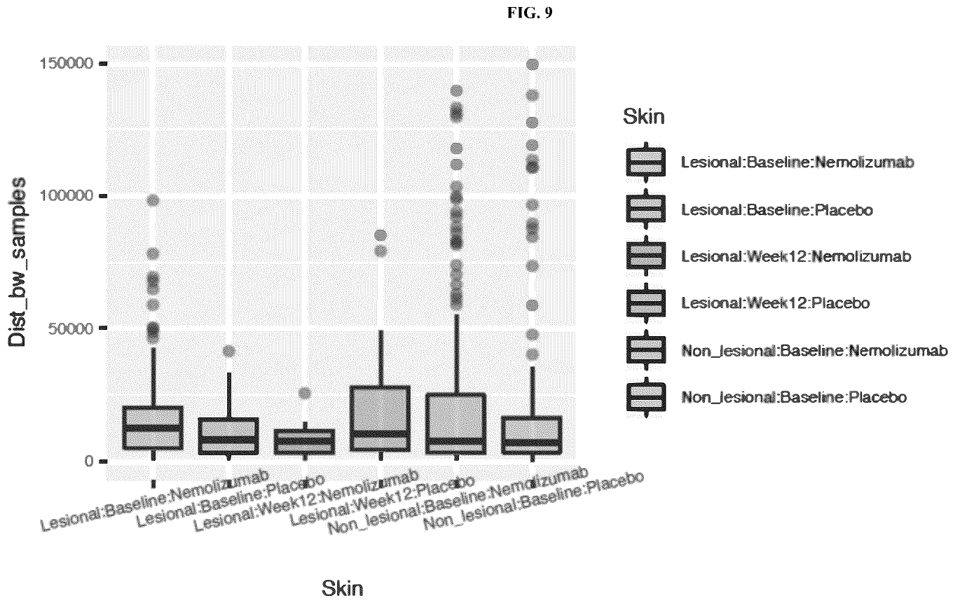

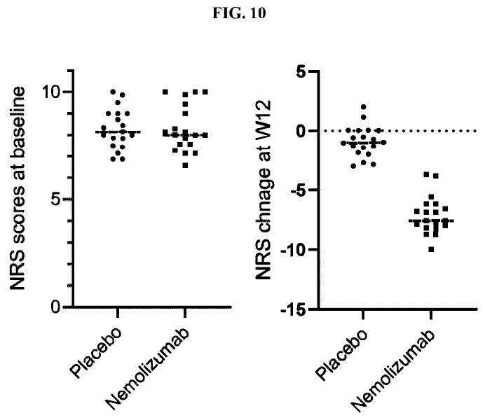

Nineteen (19) placebo non-responders and nineteen (19) nemolizumab responders were selected on the basis of their change in PP-NRS (Peak Pruritus scores on the Numerical Rating Scale) clinical scores at week 12 (19 placebo patients with PP-NRS change ≥−1.4, 19 and nemolizumab patients with PP-NRS change <−5.4). PP-NRS scores at baseline and PP-NRS after 12 weeks of nemolizumab treatment are shown in FIG. 10.

Protocol

After plasma gY14-super-depletion, samples were analyzed using the TMT calibrator MS2 workflow at Proteome Science. A pool of heathy skin biopsies was used in order to 1) allow batch correction between each plex, 2) trigger the mass spectrometer to detect skin related peptide in the blood.

Statistics

Only peptides which corresponded to a unique protein were used for the statistical analysis. Both baseline corrected, and baseline non-corrected values were used to compute differentially expressed proteins. Three statistical tests were used to compute the list of differentially expressed proteins: 1) least square regression method (with and without including patient ID), 2) robust regression method, 3) generalized regression method. 193 proteins were found differentially expressed (adjusted p-value <0.05). No additional (e.g. log FC filters) filters were applied on this protein list. Enrichment analysis was performed using the 193 proteins found differentially expressed with the use of QIAGEN IPA (QIAGEN Inc., digitalinsights.qiagen.com/IPA)).

Results

The list of 193 differentially expressed proteins was then analyzed with the enrichment software IPA. While the p-values indicate the significance of the pathways impacted in the dataset, the z-scores provide insights into the directionality, i.e., how “activated” or “inhibited” are the pathways in the dataset.

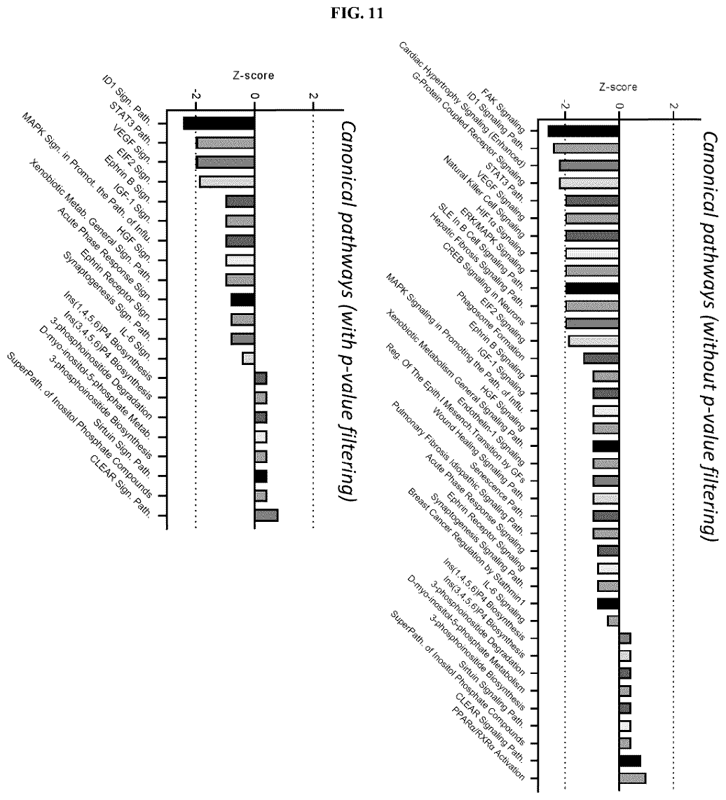

Canonical pathways were either sorted according to z-scores only (FIG. 11 right), or sorted by z-scores and filtered by p-value (FIG. 11 left; −log(p-value)>1.3 corresponding to p-value <0.05). IL-31 has been shown to induce STAT3 activation. Though STAT3 activation is not specific to IL-31, the downregulation of this pathway in nemolizumab responders suggests target engagement by nemolizumab. Further, as compared to placebo non responders, nemolizumab responder signature was characterized by the downregulation the IL-6 pathway. Additionally, the Vascular Endothelium Growth Factor (VEGF) pathway, which has been showed to correlate with PN severity, was downregulated in nemolizumab responders, possibly reflecting clinical improvement. Finally, two neuronal ontologies (“CREB signaling in neurons” and “Synaptogenesis signaling pathway”) were identified as downregulated in nemolizumab responders subjects, emphasizing the impact of IL-31 as a neuro-inflammatory cytokine in PN.

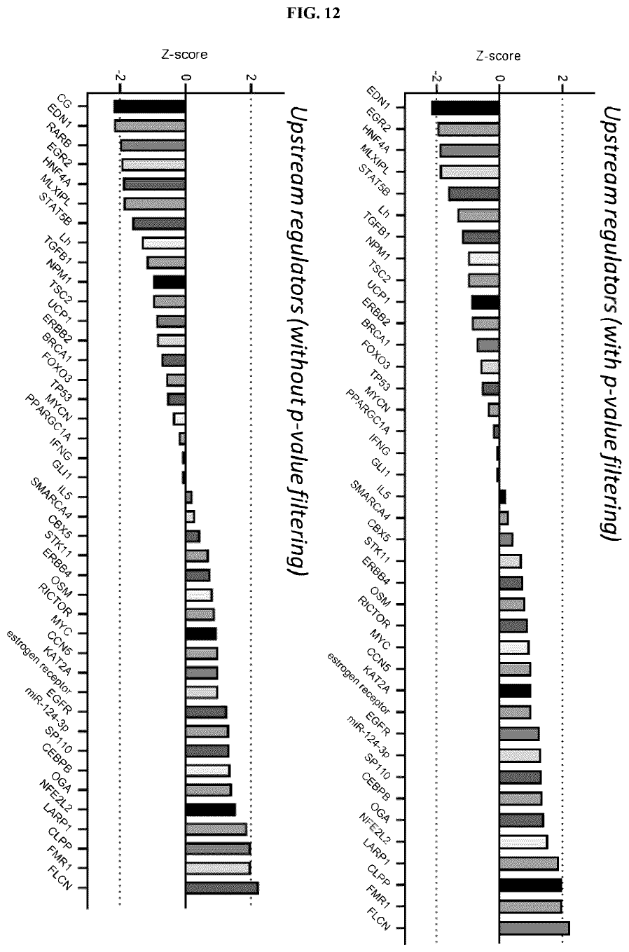

Upstream regulator analysis allowed for the identification of transcription factors and small molecules whose functions are impacted by disease or treatment depending in the dataset analyzed. Though proteins identified in this analysis may not themselves be differentially expressed in the studied dataset, their activation/inhibition status could explain the expression changes in the dataset. Canonical pathways were either sorted according to z-scores only (FIG. 12 right), or sorted by z-scores and filtered by p-value (FIG. 12 left; p-value <0.05). The pro-fibrotic Transforming Growth Factor Beta 1 (TGFβ1) was found as a potential regulator of nemolizumab responder signature, possibly indicating resolution of fibrotic nodules. Another potential regulator was Signal Transducer And Activator Of Transcription 5b (STAT5b), which may reflect target engagement by Nemolizumab. These data clearly suggest that nemolizumab strongly impacts inflammation and tissue remodeling processes in PN.

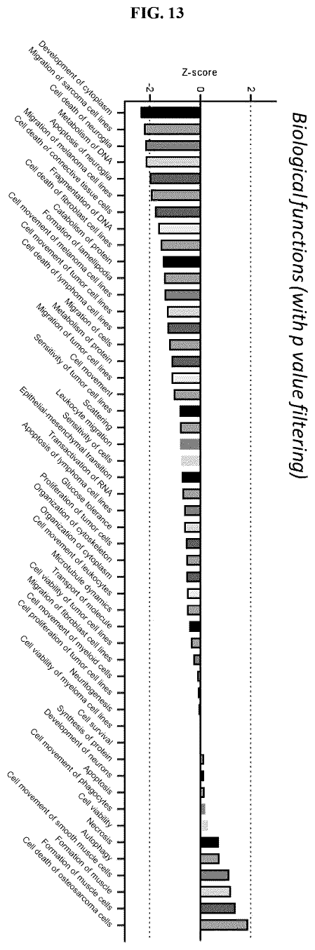

Biological function analysis of nemolizumab responders revealed downregulated “leukocyte migration” and downregulated “cell movement of leukocytes”. In addition, two terms associated with the neuronal axis were downregulated: “Cell death of neuroglia”, “Apoptosis of neuroglia”, possibly reflecting an improvement pruritus (FIG. 13).

Discussion

The present Example aimed to perform a broad and exploratory analysis to identify plasma protein biomarkers that could also explain the mechanism of action of nemolizumab treatment.

To characterize the entire gamut of plasma proteomic changes, pre-selected groups of nemolizumab responders and placebo non-responders were assessed. Due to this pre-selection of patients, the signature described in this Example may not only reflect the changes induced by nemolizumab but also the change due to decreasing PP-NRS.

The dataset analyzed in this study reveals that nemolizumab responder signature was characterized by an improvement of various aspects of PN pathophysiology including inflammation, neuroimmune function, and tissue remodeling.

Enrichment analysis reveals that the nemolizumab responder signature was characterized by a decrease of migration and cell movement of leukocytes. The STAT3 pathway, a direct target of IL-31 signaling was also inhibited in nemolizumab responder subjects suggesting target engagement. Nemolizumab responders also displayed downregulation of other pro-inflammatory cytokine pathways including IL-6 and VEGF pathways.

Pathway analysis also revealed the impact of nemolizumab treatment on neuron-associated processes including “(“CREB signaling in neurons” and “Synaptogenesis signaling pathway”, “Cell death of neuroglia”, “Apoptosis of neuroglia”, in line with the observed clinical impact of nemolizumab on pruritus.

Finally, the data set showed a decrease in TGFB1 pathway activation suggesting an impact of nemolizumab treatment on tissue remodeling.

These observations reinforce the understanding that plasma proteomics can effectively capture, at least in part, tissue specific (i.e., skin) effects of nemolizumab in PN.

### Example 3—Fibroblast Involvement in PN Pathogenesis

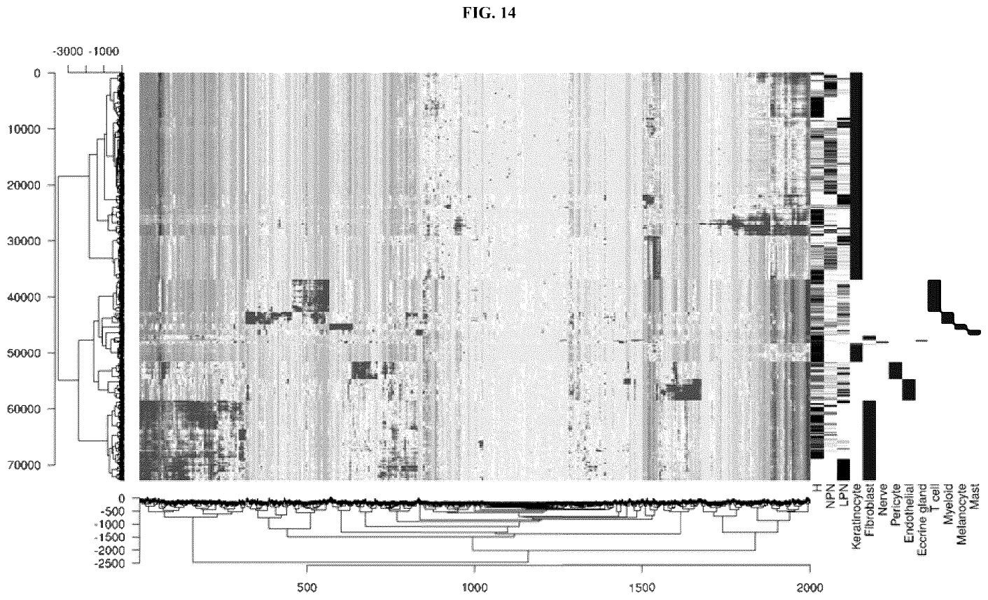

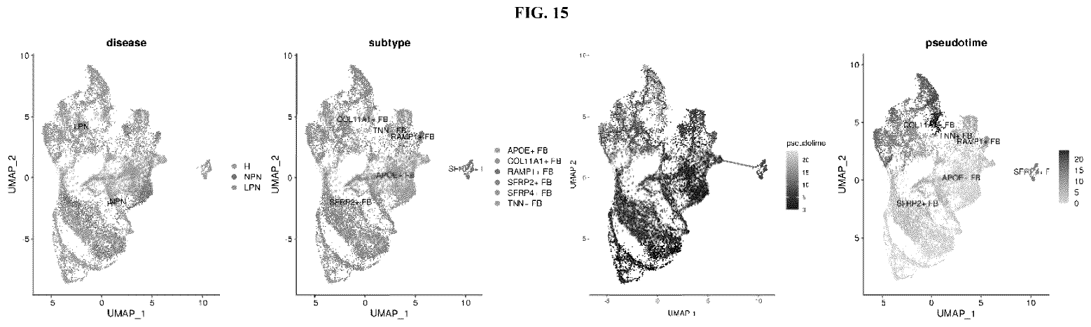

Single-cell and bulk RNA sequencing (RNAseq) were combined to delineate molecular and cellular signatures of PN and investigate the impact of nemolizumab in treated patients. Single-cell RNAseq (scRNAseq) was performed on skin biopsies obtained from 4 patients with PN and 4 healthy controls (FIGS. 14 and 15). The role of fibroblasts in PN pathogenesis was further investigated via a cell-cell communication analysis using CellChat. Bulk RNAseq was performed on skin biopsies obtained at baseline and week 12 from a nemolizumab phase 2 study in patients with PN (n=70).

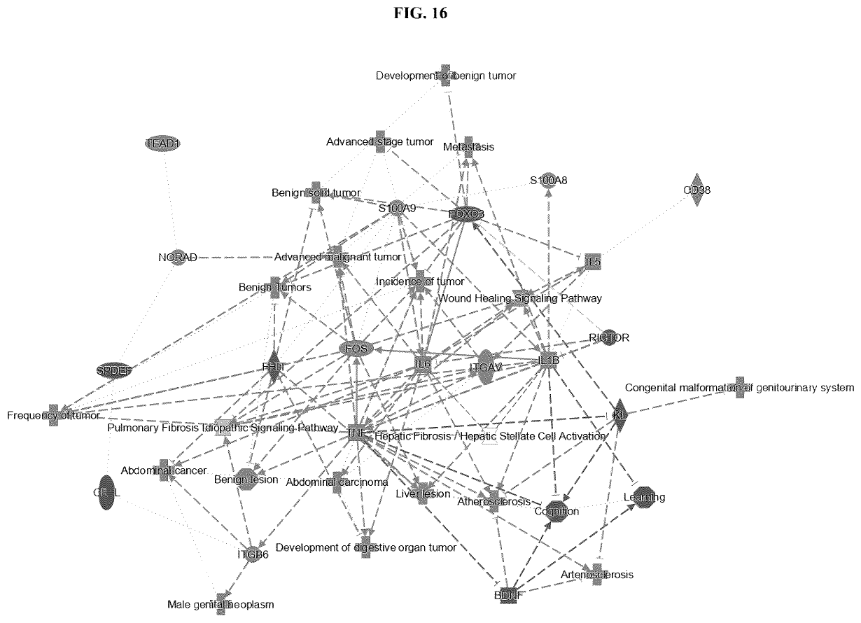

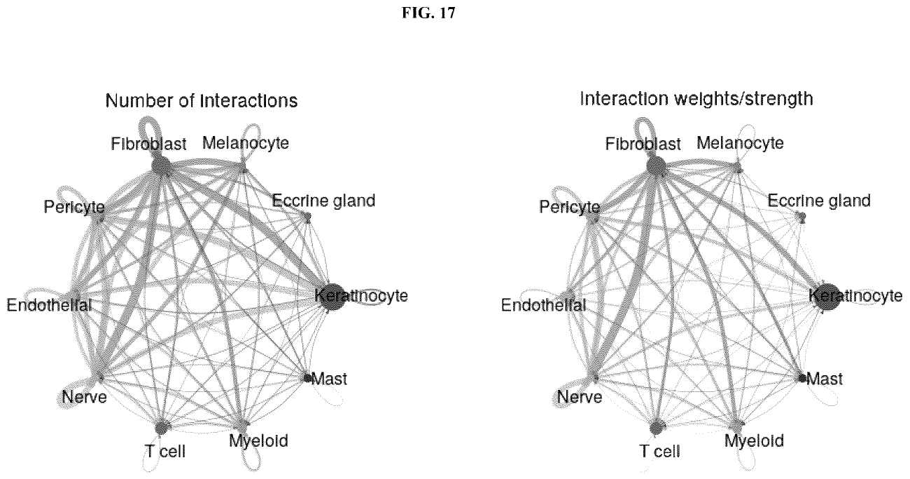

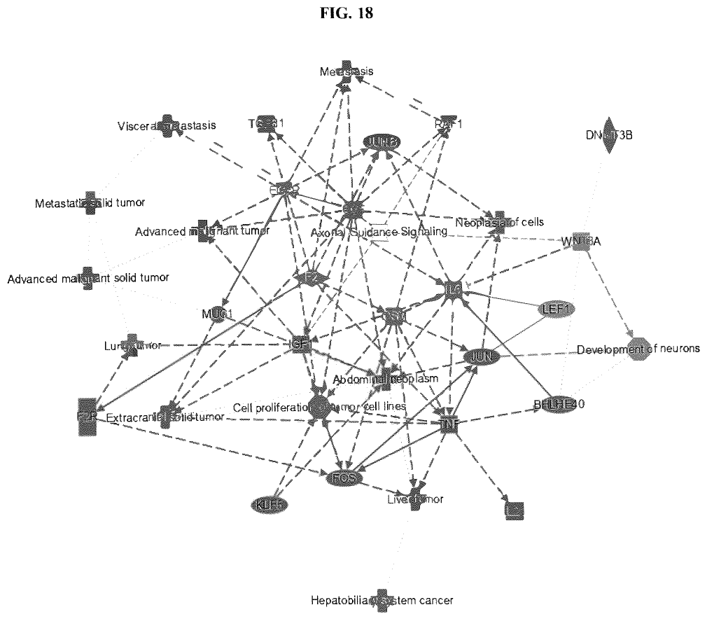

scRNAseq results showed that compared to healthy skin, lesional PN fibroblasts take on a pro-fibrotic and pro-inflammatory state, confirmed by trajectory analyses, reflecting altered differentiation of fibroblasts in PN skin (FIG. 16). Consistent with this, functional analysis of differentially expressed genes in lesional PN fibroblasts indicates activation of inflammatory (TNF, IL1B, IL6) and pro-fibrotic (TGFβ) signaling pathways. CellChat results highlight the role of the PN fibroblast as a central player in the intra-cellular cross-talk (FIG. 17). Combining bulk and single-cell RNAseq data, it was observed that nemolizumab restores neural dysregulation and reduces inflammation and fibrosis (FIG. 18).

The this study demonstrated that PN is an inflammatory and fibrotic disease and that nemolizumab exerts an anti-fibrotic effect via inhibiting critical signaling pathways.

### Example 4—scRNA-Seq in Prurigo Nodularis (PN)

### Abstract

Background: Prurigo nodularis (PN) is a chronic neuroimmune skin disease characterized by symmetrically distributed pruritic hyperkeratotic nodules on extremities and trunk. Neuroimmune dysregulation and chronic scratching are believed to both induce and maintain the characteristic lesions.

Objective: This example provides a comprehensive view of the molecular pathogenesis of PN at the single-cell level to identify and outline key pathologic processes and the cell types involved. Features that distinguish PN skin from the skin of patients with atopic dermatitis (AD) were of particular interest. A further aim was to determine the impact of the interleukin-31 (IL-31) receptor alpha antagonist, nemolizumab, and its specificity at the single-cell level.

Method: Single-cell RNA-sequencing (scRNA-seq) of skin from 15 healthy donors and nonlesional and lesional skin from 6 patients each with PN and AD, combined with spatial-sequencing (spatial-seq) using the 10× Visium platform. Integration with bulk RNA-seq data from patients treated with nemolizumab.

Results: The results described in this example demonstrate that PN is an inflammatory skin disease characterized by both keratinocyte proliferation and activation of profibrotic responses. This example demonstrates that the COL11A1+ fibroblast subset is a major contributor to fibrosis and is predominantly found in the papillary dermis of PN skin. Activation of fibrotic responses is the main distinguishing feature between PN and AD skin. This example further shows the broad effect of nemolizumab on PN cell types, with a prominent effect driving COL11A11+ fibroblast and keratinocyte responses toward normal.

Conclusion: This example provides a high-resolution characterization of the cell types and cellular processes activated in PN skin, establishing PN as a chronic fibrotic inflammatory skin disease. It further demonstrates the broad effect of nemolizumab on pathological processes in PN skin.

### Introduction

Prurigo nodularis (PN) is a chronic neuroimmune skin disease characterized by chronic and intense pruritus with a marked impact on quality of life. Clinically it is characterized by multiple nodules that can cover large areas of the extremities and the trunk. The pathogenesis of PN remains unclear, but previous studies have implicated immune and neural dysregulation as critical circuits in its pathogenesis. PN has been suggested to have a clinical and pathologic overlap with atopic dermatitis (AD) by which it may share a T helper 2 (Th2) polarization. However, a direct comparison of these two conditions has not yet been performed at a single-cell level. Indeed, in a previous study, Bulk-RNASeq analyses revealed that PN has a distinct molecular signature as compared to AD. The best characterized immune mediator in PN is the cytokine IL-31 receptor alpha (IL-31RA) and oncostatin M receptor beta (OSMRB) Activated Th2 cells are considered the main IL-31 source, but other cell types can also produce IL-31, including eccrine sweat glands, mast cells, basophils, eosinophils, monocytes/macrophages. A recent study in human PN lesions revealed that macrophages, in addition to T cells, are major cellular sources of IL-31. The critical importance of IL-31 to PN pathogenesis has been demonstrated with nemolizumab, an investigational monoclonal antibody that inhibits IL-31RA, which leads to effective suppression of downstream inflammatory responses, including Th2 responses and stabilization of extracellular matrix (ECM) remodeling. Recently published evidence has indicated that fibrosis is a feature of PN, but the source of this fibrosis in PN skin is unknown. The major fibroblast populations in human skin have been described with the two major clusters characterized by the expression of SFRP2 and FMO1, with five minor fibroblasts defined including COL11A1+ fibroblast, with three (SFRP2+, FMO1+, and COL11A1+), based on their gene expression profiles, suggested to have a role in matrix deposition, inflammatory cell retention, and connective tissue cell differentiation.

This example provides an in-depth exploration of PN pathogenesis to characterize the central mechanisms involved and identify the target cells of nemolizumab treatment, and contrast its pathogenesis with AD through a combination of single-cell RNA (scRNA) and spatial-RNA-sequencing approaches.

### Results

Single Cell RNA-Seq and Spatial-Seq Reveal Diverse Cell Types and their Spatial Locations in PN Skin

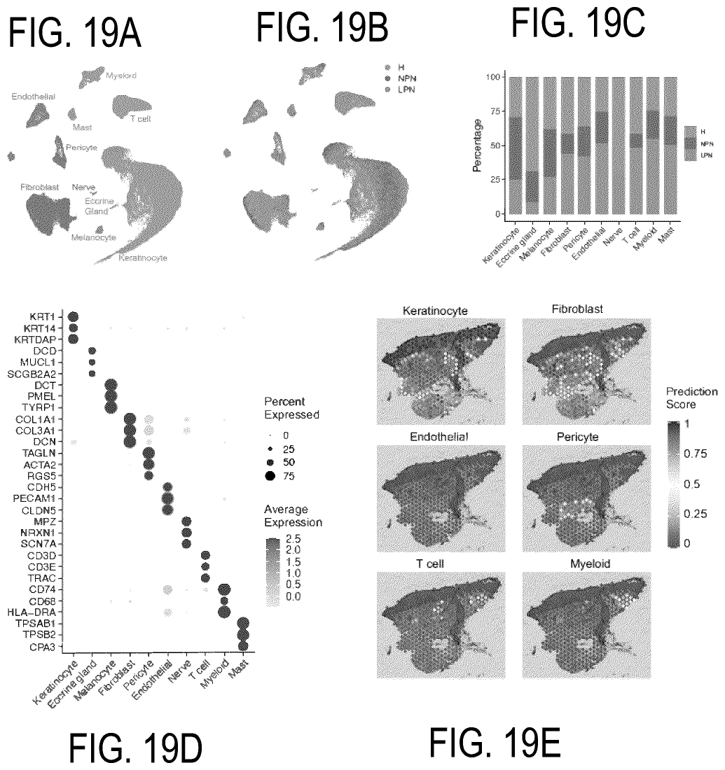

To understand the unbiased cellular composition and cell states of healthy (H) skin and lesional PN (LPN) skin, single-cell suspensions of skin biopsies from 15 healthy donors and 6 patients with PN were generated. Skin biopsies were also collected from peripheral nonlesional sites from 4 out of the 6 PN patients (NPN), yielding a total 25 scRNA-seq libraries. The resulting quality-controlled PN plus healthy single-cell atlas contained a total of 72,782 cells, with an average of 2,379 genes and 10,417 transcripts detected per cell. To study the heterogeneity of these cells, variable genes were selected and used to perform uniform manifold approximation and projection (UMAP) dimensionality reduction and cell clustering using the R package Seurat. Cluster annotation was corroborated by overlapping the cluster markers with the canonical lineage-specific genes reported in previous skin disease scRNA-seq studies. Ten major cell types were recovered across all the samples (FIG. 19A), including keratinocytes, melanocytes, eccrine gland cells, endothelial cells, fibroblasts, pericytes, nerve cells, T cells, myeloid cells, and mast cells. Most of these cell types contained cells from the majority of the healthy, NPN, and LPN libraries, suggesting that each cell type was associated with a common cell lineage rather than derived from a specific condition. Two small cell populations, eccrine gland cells and nerve cells were mainly derived from healthy samples. Interestingly, clear separations were observed in the keratinocytes, fibroblasts, and endothelial cells among the healthy, NPN, and LPN cells, suggesting major transcriptional differences (FIG. 19B). Moderate shifts in terms of the proportion of cell types were seen in LPN compared to NPN and healthy, with the most pronounced shifts in mast cells, endothelial cells, T cells, and myeloid cells in LPN skin (FIG. 19C). Marker genes for each cell population showed a clear separation between each cell type (FIG. 19D).

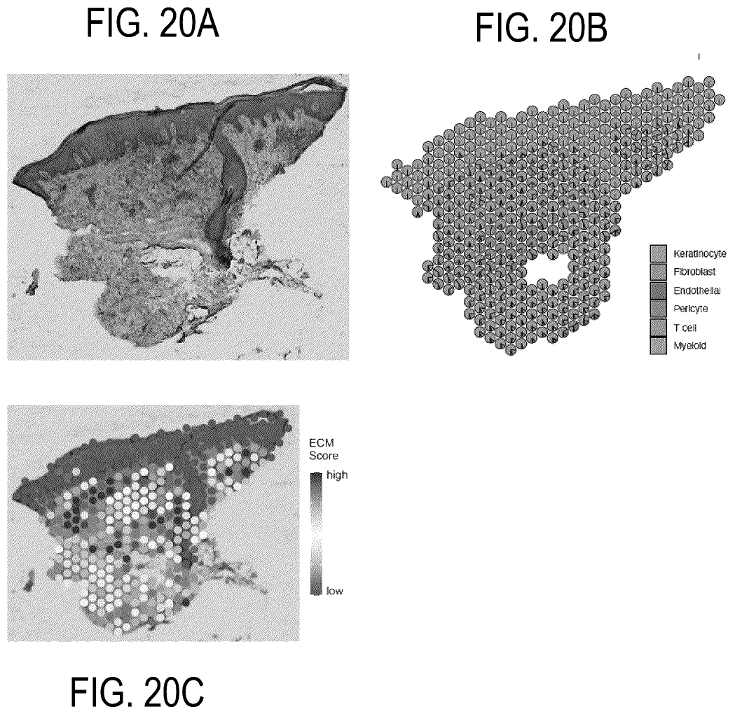

To localize the major cell types detected by scRNA-seq in Systemic sclerosis (SSc) skin, spatial sequencing (spatial-seq) was performed on the SSc skin sample using the 10× Visium platform. 395 spatially defined spots were detected with an average of 2,613 genes and 4,432 transcripts per spot (FIG. 20). The spatial spots were deconvoluted by the major cell types detected in scRNA-seq using the Seurat anchor-based label transfer method. The deconvoluted prediction scores for each cell type were displayed on the tissue (FIG. 19E) and combined into a scatter-pie plot representing the relative cell type composition for each spot (FIG. 20B). Keratinocytes localized to the epidermis and the hair follicle. Myeloid cells and T cells were primarily located in the superficial dermis in proximity to the epidermis. Fibroblasts were distributed throughout a large proportion of the spots in the dermis, and the pericytes were located close to the blood vessels (FIG. 20B). These two cell types were the main producers of extracellular matrix (ECM) components (FIG. 20C). The other cell types represent small populations and were lowly detected in the spatial-seq sample.

### COL11A1+ Fibroblasts are Enriched for Pro-Fibrotic Responses in PN Skin

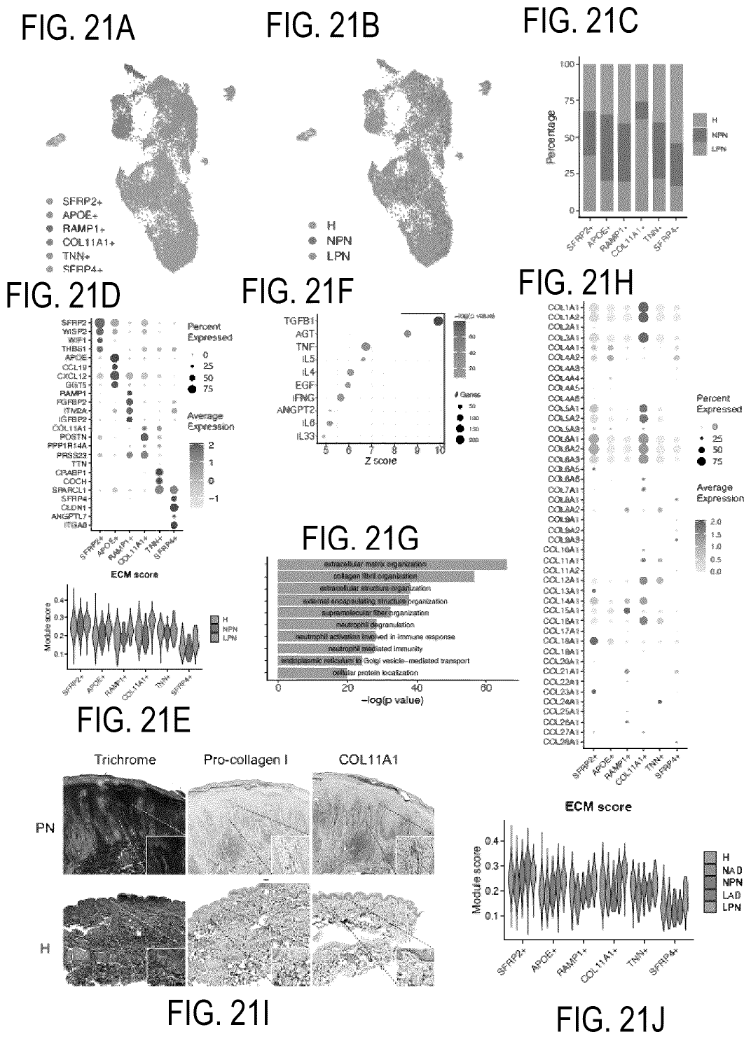

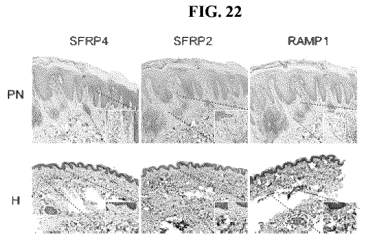

To characterize the heterogeneity of the fibroblasts, all the fibroblasts from the scRNA-seq dataset were sub-clustered. Based on previously published marker genes, the fibroblast sub-clusters were annotated into six subtypes, including SFRP2+ fibroblasts (FB), APOE+FB, RAMP1+FB, COL11A1+FB, TNN+FB, and SFRP4+FB (FIG. 21A and FIG. 20). Interestingly, COL11A1+FB were mainly derived from the LPN samples compared to the healthy or NPN samples (FIG. 21B, C). The COL11A1+FB expressed high levels of COL11A1, POSTN, and PRSS23, suggesting a profibrotic role in PN skin (FIG. 21D). To illustrate the capacity of ECM production by the different fibroblast subtypes, the ECM module score was calculated using the gene list from the extracellular matrix pathway from Gene Ontology and identified the highest ECM score in the LPN COL11A1+FB (p=4.4E-83) (FIG. 21E). Differential expression analysis was then performed between the LPN and healthy COL11A+FB and inferred the upstream regulators driving the differential expression. High activation z-score of transforming growth factor beta-1 (TGFB1), IL-5, and IL-4 are likely reflective of pro-fibrotic responses, and high tumor necrosis factor (TNF), interferon gamma (IFNG), and IL-6 activation z-scores suggest inflammatory response in the LPN COL11A1+FB compared to healthy counterparts (FIG. 21F). Enrichment analysis also revealed top fibrosis-related pathways (i.e., extracellular matrix organization, collagen fibril organization) and inflammation-related pathways (i.e., neutrophil degranulation, neutrophil activation involved in immune response) (FIG. 21G). The COL11A1+FB had the highest expression pattern of the collagen genes, including COL1A1, COL1A2, COL3A1, COL5A1, COL5A2, COL6A1, COL6A2, COL6A3, COL11A1, COL12A1, COL14A1, and COL16A1 (FIG. 21H). Taken together, the above results suggest a strong fibrotic potential for COL11A1+FB in LPN skin. The presence of the major fibroblast subtypes (SFRP4, SFRP2, RAMP1, and COL11A1 was confirmed (FIG. 21I and FIG. 22) and validated the fibrosis phenotype by immunohistochemistry in PN skin. To compare the fibrosis-inducing capacity of the fibroblasts in PN and AD, PN scRNA-seq datasets were combined with a single-cell dataset from AD skin and compared ECM scores among the fibroblast subtypes in healthy (H), nonlesional AD (NAD), NPN, lesional AD (LAD), and LPN skin. Although the LAD fibroblast subtype exhibited a higher ECM score compared to healthy or peripheral nonlesional fibroblasts, LPN fibroblast subtypes expressed a strikingly higher ECM score than the LAD cells (p=1.8×10-15) (FIG. 21J), particularly in COL11A1+ FBs which was the FB subset most prominently increased in PN skin (p=2.2×10⁻⁹).

### Endothelial Cells and Pericytes Demonstrate Fibrotic and Inflammatory Responses in PN Skin

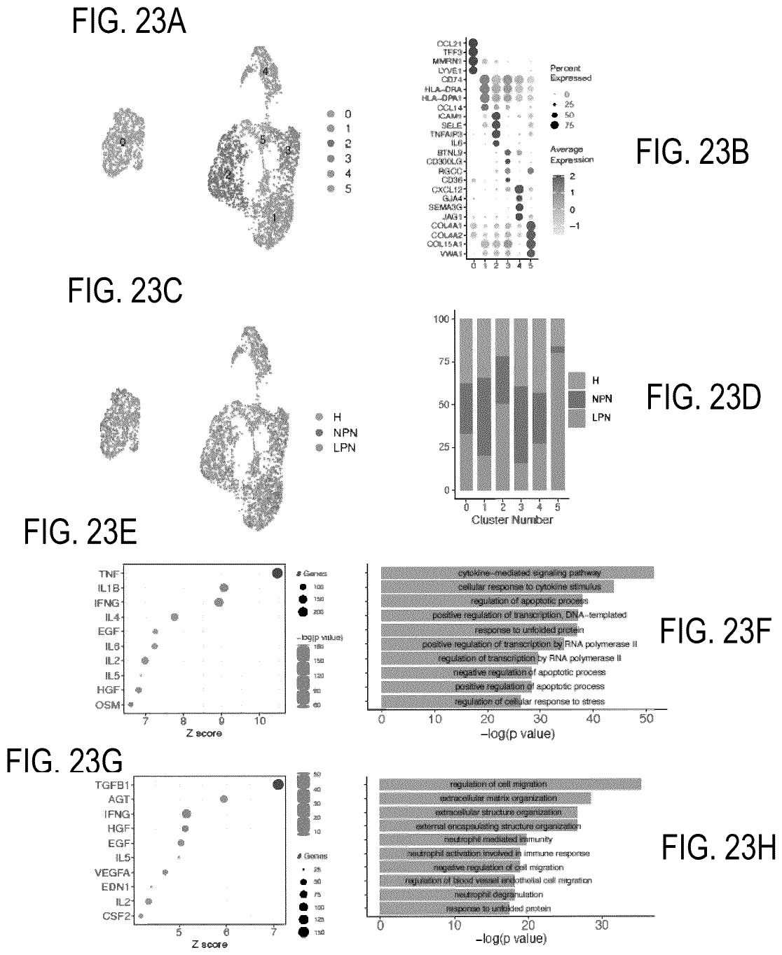

Next, the heterogeneity of the endothelial cells was investigated and these cells were clustered into six sub-clusters (FIG. 23A, B). Disease composition analysis identified that endothelial sub-cluster 2 and 5 were enriched in LPN compared to healthy or NPN samples (FIG. 23C, D) (p=0.0014 and 2.1×10⁻²², respectively). Sub-cluster 2 represented the activated endothelial cells with high expression of ICAM1 and E-selectin (SELE), which also display inflammatory features such as TNFAIP3 and IL6. Sub-cluster 5 expressed high levels of several collagen genes (i.e., COL4A1 and COL15A1), suggesting potential involvement in fibrosis (FIG. 3B). To study these two LPN-specific sub-clusters, enrichment analysis was performed using their cluster marker genes. Sub-cluster 5 marker genes implicated proinflammatory cytokines (i.e., TNF, IL1B, IFNG, IL6) and pathways (i.e., cytokine-mediated signaling pathway, cellular response to cytokine stimulus) (FIG. 23E, F). Sub-cluster 2 marker genes were regulated by profibrotic upstream regulators (i.e., TGFB1, angiotensinogen (AGT), epidermal growth factor (EGF), IL-5) and enriched in ECM-related pathways (i.e., extracellular matrix organization, extracellular structure organization) (FIG. 23G, H). These results suggest that endothelial cells were actively involved in both fibrotic and inflammatory responses in PN skin.

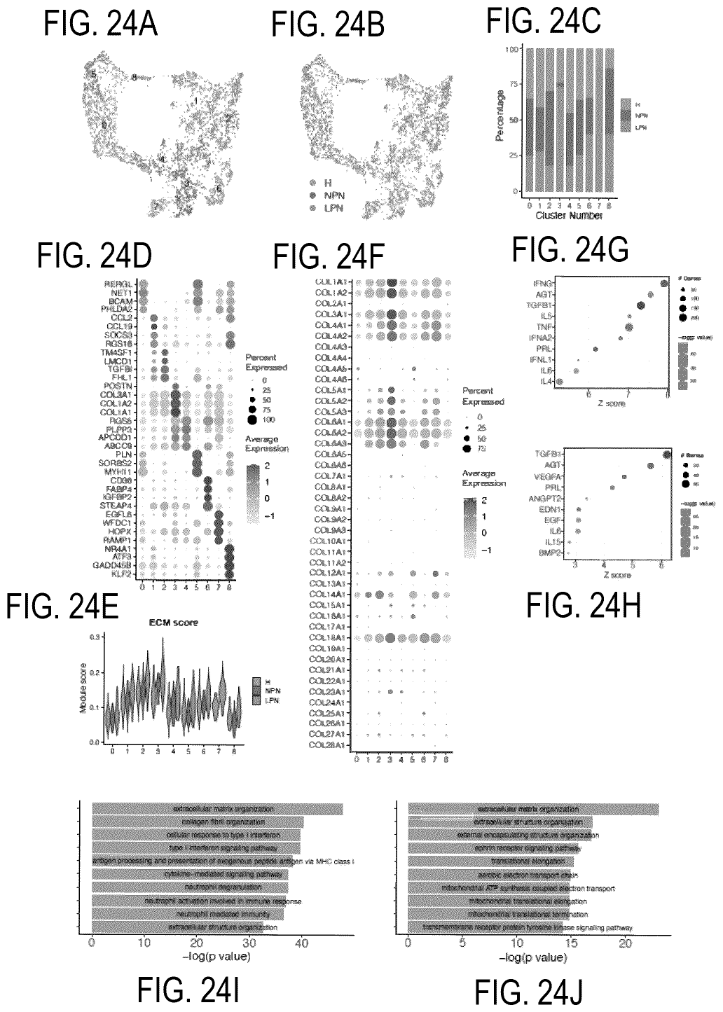

Similarly, the pericytes were sub-clustered to obtain nine sub-clusters (FIG. 24A, D). Composition analysis identified that sub-cluster 3 and sub-cluster 8 were enriched in LPN samples compared to healthy or NPN samples (FIG. 24B, C). The ECM score was calculated and plotted all the collagen genes across the pericyte subtypes. Cells in sub-cluster 3 had the highest ECM score in LPN and displayed the highest expression pattern of the collagen genes (FIG. 24E, F). LPN cells in sub-cluster 7 also revealed a much higher ECM score than the healthy cells, and expressed the second-highest pattern of collagen genes (FIG. 24E, F). Enrichment analyses was then performed using the marker genes for sub-cluster 3 and sub-cluster 7 and found that both sub-clusters implicated in profibrotic upstream regulators (i.e., TGFB1, AGT, prolactin (PRL)) and pathways (i.e., extracellular matrix organization, collagen fibril organization) (FIG. 22G-J). Sub-cluster 3 also illustrated inflammatory response driven by interferon (FIG. 22G, I). Taken together, these results demonstrate that endothelial cells and pericytes may actively contribute to fibrotic and inflammatory responses in lesional PN skin.

### Keratinocyte Responses in PN Skin

The keratinocytes were sub-clustered to obtain six keratinocyte subtypes: basal, spinous, supraspinous, granular, follicular, and inflammatory keratinocytes (FIG. 25A, D). The inflammatory keratinocytes were primarily derived from LPN samples (FIG. 25B, C). Enrichment analysis using the inflammatory keratinocyte markers implicated proinflammatory upstream regulators and mitochondrial respiratory pathways, suggesting high energy consumption in PN keratinocytes during inflammation (FIG. 25E). Upstream regulators of the inflammatory subtype of keratinocytes included the Th2 cytokines (IL-4, IL-5, IL-33), along with TGFB1 (FIG. 25F), supportive of an enriched Th2 response in lesional PN skin.

scRNA-Seq Reveals Immune Subtype Heterogeneity in PN Skin

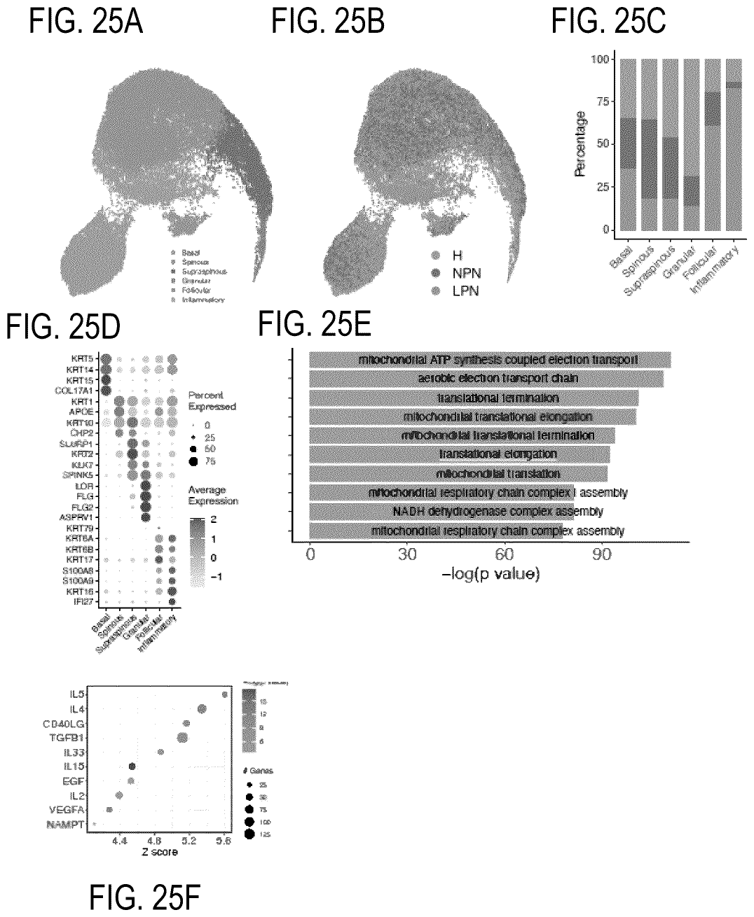

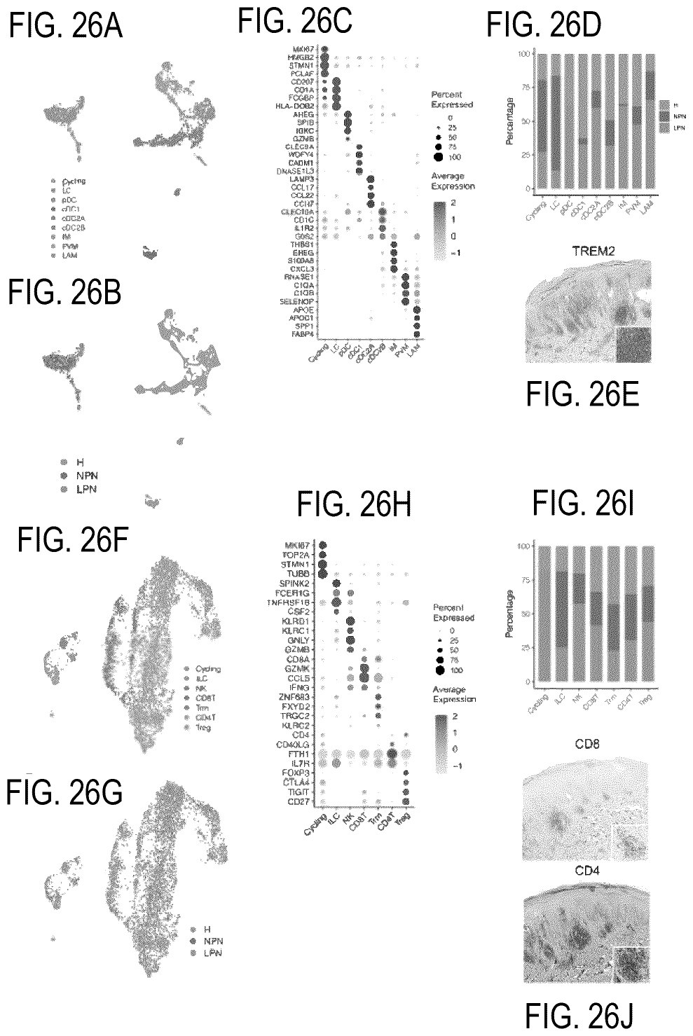

Given the strong inflammatory response observed in fibroblast, endothelial cells, and pericytes, the heterogeneity of immune cells was investigated. The myeloid cells were sub-clustered and annotated into nine subtypes, including cycling myeloid cells, Langerhans cells (LC), plasmacytoid dendritic cells (pDC)−, classical type 1 dendritic cells (cDC1), classical type 2 dendritic cells subset A (cDC2A), classical type 2 dendritic cells subset B (cDC2B), interstitial macrophages (IM), perivascular macrophage, and lipid-associated macrophages (LAM, also called TREM2 macrophage) (FIG. 26A-E) p-values for pDC, cDC2A, IM, PVM, and LAM were 1.30×10-13, 3.33×10-8, 1.72×10-12, 9.41×10-5, and 4.5×10-11, respectively). An increased proportion of pDCs, cDC2A, and macrophage subpopulations (IM, PVM, and LAM/TREM2) was observed in lesional PN skin compared to non-lesional and healthy (FIG. 26D-E). The prominence of LAM/TREM2 macrophages in lesional PN skin was confirmed using immunohistochemistry (FIG. 26). For T cells and other lymphoid cells, seven subtypes were obtained: cycling T cells, innate lymphoid cells (TLC), natural killer cells (NK), CD8+ T cells (CD8T), tissue-resident memory T cells (Trm), CD4+ T cells (CD4T), and regulatory T cells (Treg). Several NK and T cell populations showed increased proportion in lesional PN skin including cycling, NK, CD8, and T regulatory cells. The presence of T cells in lesional PN skin by IHC staining for CD8 and CD4 was confirmed (FIG. 26).

### Ligand-Receptor Analysis Reveals Cell Type-Specific Networks in PN

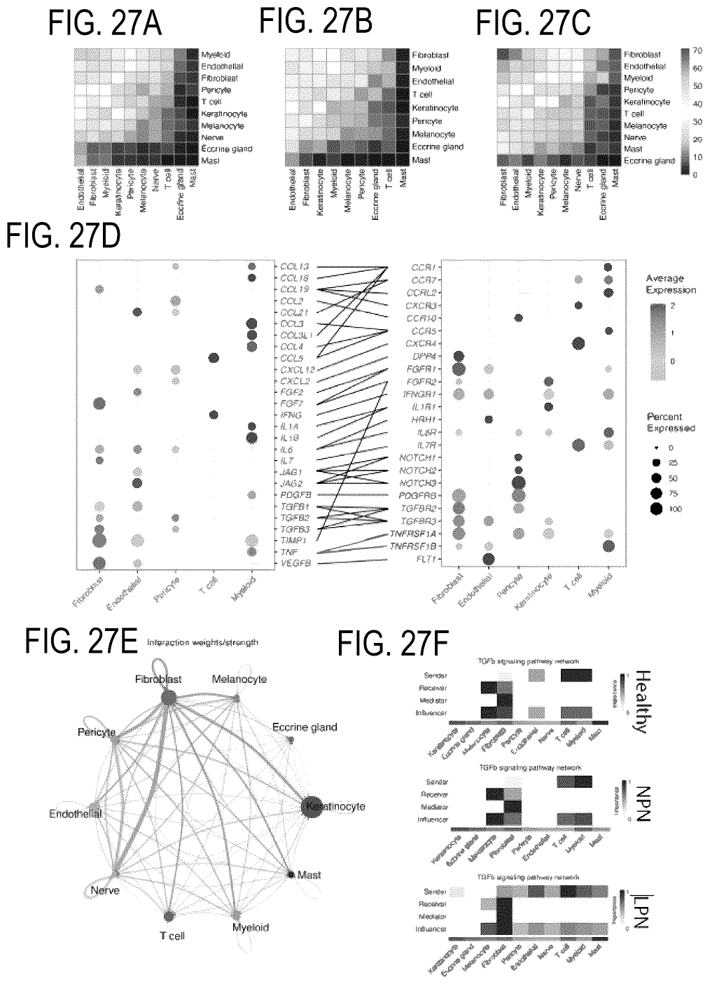

To address the observed shifts in cell type composition and transcriptional changes, cell-cell communication changes were analyzed in PN compared to healthy skin. To do this, separate ligand-receptor analyses was performed on the healthy, NPN, and LPN cell types using CellphoneDB and CellChat. The greatest number of interactions were observed in LPN (FIG. 5C) compared to the healthy (FIG. 27A) or NPN skin (FIG. 27B), particularly among fibroblasts, endothelial cells, pericytes, myeloid cells, and keratinocytes. To study the specific ligand-receptor pairs in PN, the pairs that had higher interaction scores in LPN compared to healthy or NPN were selected, which uncovered various signaling pathways implicated in PN arising from both immune (T cell, myeloid) and stromal cell populations (fibroblasts, endothelial cells, and pericytes). Several validated proinflammatory cytokines, such as IFNG, IL1, IL6, and TNF, were also involved in PN pathogenesis. Notably, this analysis revealed several other proinflammatory mediators, including CCL2, CCL3, CXCL2, CXCL12, and IL7, that were expressed by various cell types in PN skin (FIG. 27D, E). Emergence of fibroblast growth factor (FGF2, FGF7), platelet-derived growth factor (PDGFB), transforming growth factor beta (TGFB1, TGFB2, TGFB3), and vascular endothelial growth factor (VEGFB) corroborated involvement of fibrosis in PN pathogenesis. A robust signaling network was observed related to the TGFB signaling pathway in lesional PN skin with the source of TGFB observed in multiple cell types and the main target cell in LPN skin being fibroblasts (FIG. 27F). Taken together, these data illustrate profibrotic and proinflammatory shifts within the interactome in PN skin.

### Comparison Between Epithelial Responses in PN Versus AD Skin by Single-Cell Analyses

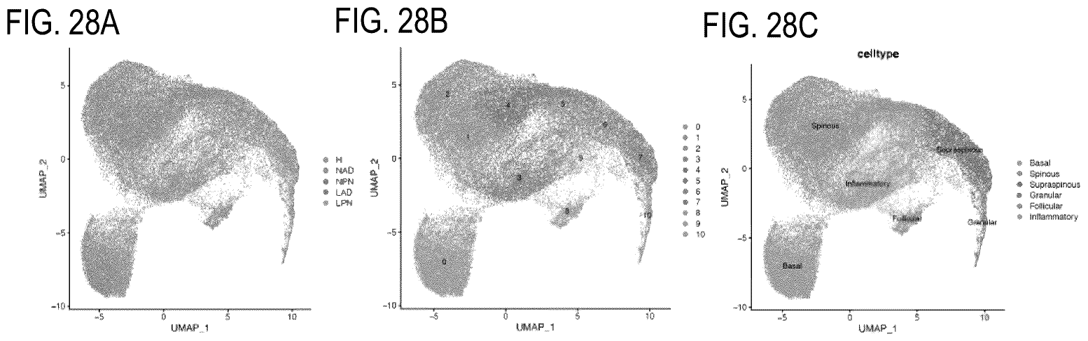

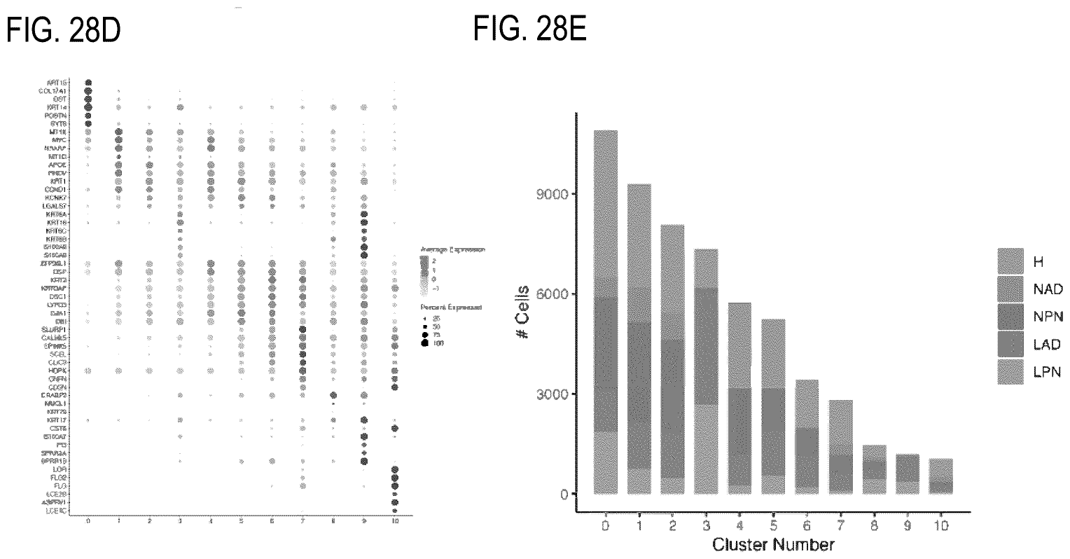

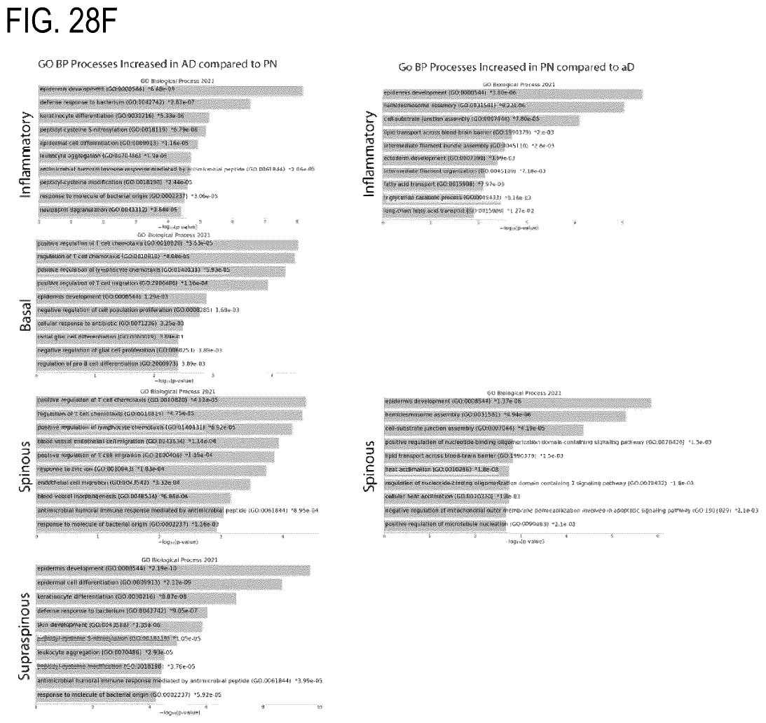

As mentioned previously, lesional PN skin showed an expansion of COL11A1 fibroblasts as compared to lesional AD skin, suggesting a more pro-fibrotic signature in PN pathophysiology (FIG. 21J and FIG. 22B). Keratinocyte responses were further compared in PN and AD skin and compared gene expression changes in each compartment (FIG. 28A-D). Both PN and AD skin had an expansion of KCs in cluster 3 which corresponded to an “inflammatory” phenotype with prominent expression of several inflammatory markers including KRT6 and KRT16, S100A8 A9, along with IFN signature genes such as IFI27, IFITM3, and the inflammasome gene PYCARD encoding apoptosis-associated speck-like protein containing a CARD (ASC) (FIG. 28A,D,E). Assessing enriched gene ontology categories in each KC compartment using a threshold of FC>2, and FDR<0.05, demonstrated fairly consistent changes across the “inflammatory”, basal, spinous, supraspinous, and follicular keratinocytes, with AD having enriched inflammatory responses such as “defense response to bacterium” and “neutrophil degranulation” (p=2.8×E-07 and p=3.8×E-05, respectively), in “inflammatory” KCs), and T-cell chemotaxis in basal, and spinous AD KCs (p=3.5×10E-05, p=4.1×10E-5, respectively) compared to PN skin (FIG. 28F). In contrast, terms associated with altered epidermal differentiation were enriched in “inflammatory” and spinous keratinocytes from PN as compared to AD skin (FIG. 28F). Consistent in all KC subtypes was higher expression of the chemokine CCL27, S100A7, and S100A9 in AD compared to PN.

### Comparison of Immune Cell Responses in PN Versus AD Skin by Single-Cell Analyses

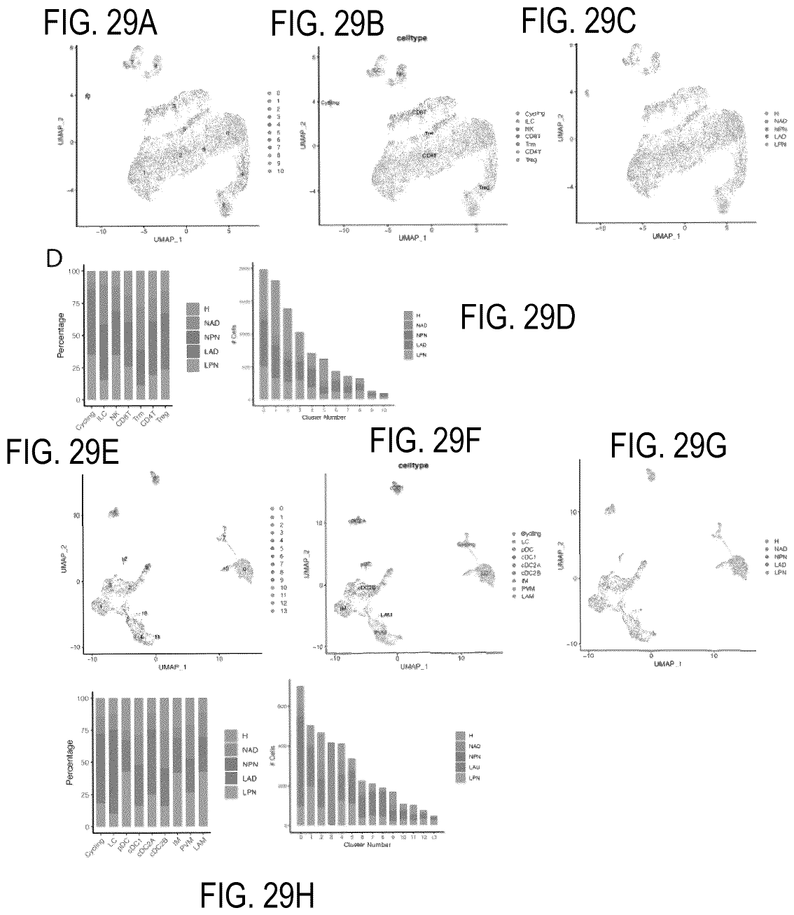

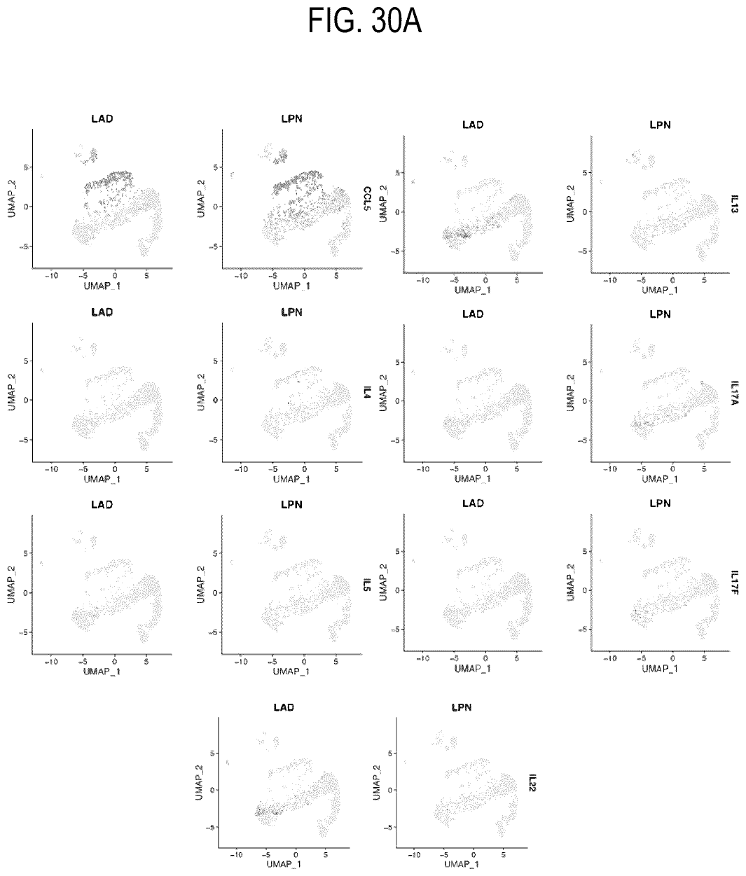

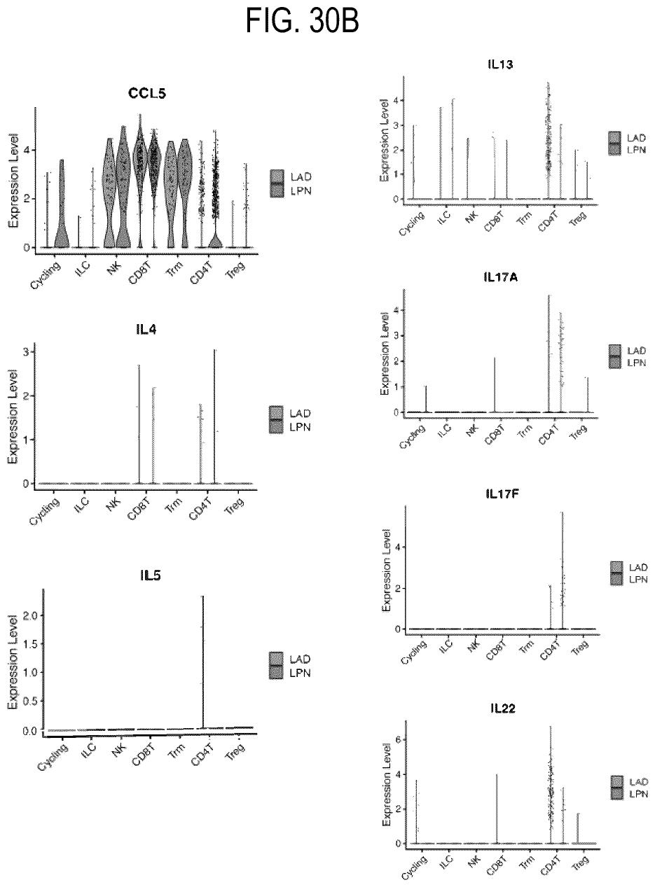

The proportion of T cell subsets was overall similar between PN and AD skin, across several T cell subsets including CD8, and CD4 effector T cells, Tregs, and cycling T cells. In addition, prominent ILC and NK cell subsets were present in both NPN, LPN, NAD, and LAD skin (FIG. 29A-D). Most prominent differences between LAD and LPN skin were found in CD4+ effector T cells, with LAD CD4⁺ cells having increased expression of IL13 (2.7-fold higher, FDR=8.5×10E-24), and IL22 (4.7-fold, FDR=6.0×E10-21). Very Few IL4 positive T cells were observed in LAD skin (FIG. 30). In contrast, LPN CD4+ T cells had higher expression of CCL5 (2.4-fold, FDR=2.8×10E-16). There was a trend towards increased expression of IL17A and IL17F in LPN compared to LAD skin, but this was not significant (FIG. 30).

The same 9 myeloid cell subsets were identified as above in PN, AD, and healthy skin with several subsets being more prominent in LPN skin compared to LAD (FIG. 29E-H). This included pDCs, interstitial macrophages (IM), and lipid-associated macrophages (LAM, TREM2⁺). Most prominent differences in gene expression were found only in PN IM macrophages, which had increased expression of CCL3 and CCL4 (2.7-fold for both, FDR=2.0×10E-09 and 3.9×10E-05, respectively). In contrast LAD IM macrophages had increased expression of MHC class II molecules including HLA-DRB1, HLA-DQA1, and HLA-DQB1 (2.2-, 2.3-, and 2.7-fold, and FDR=2.7×10E-10, =8.6×10E-14, =9.8×10E-13, respectively).

IL-31 Receptor Alpha Blockade with Nemolizumab Reverts the Transcription Profile in LPN Fibroblasts and Keratinocytes Toward Healthy

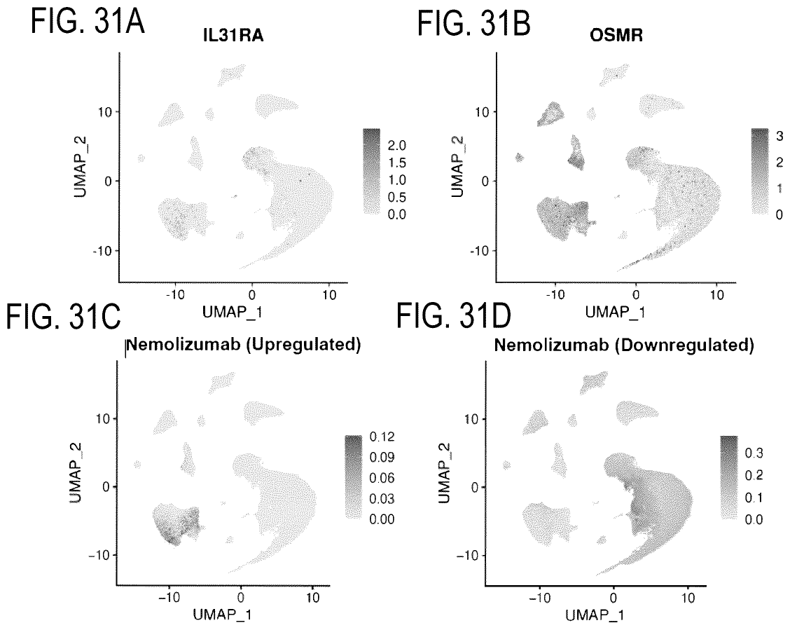

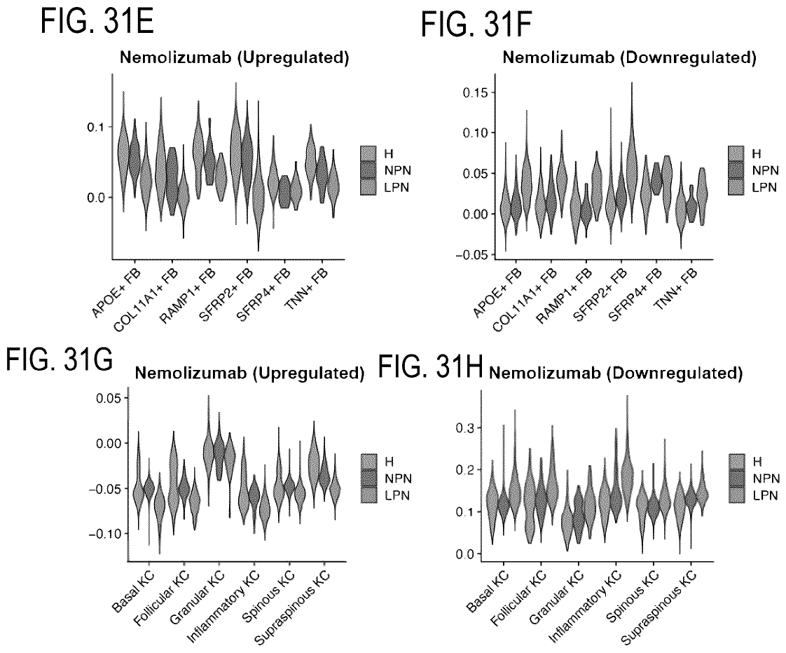

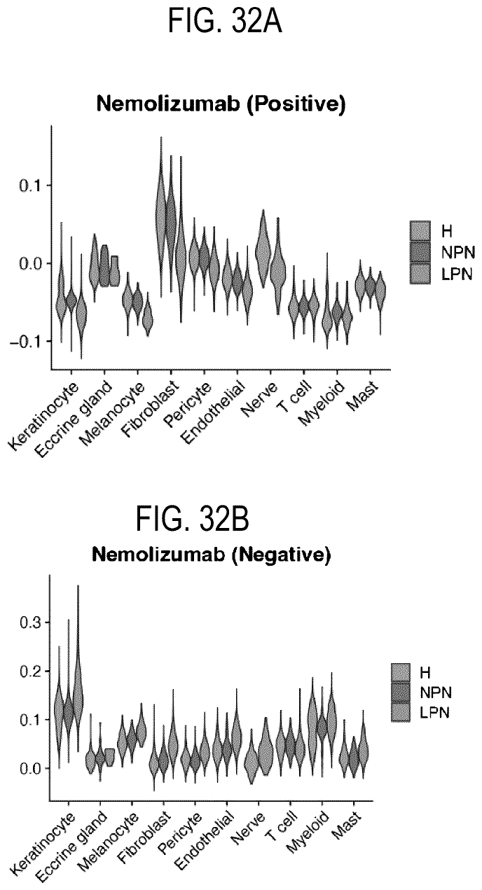

It was demonstrated that transcriptomic changes using a bulk RNA-seq approach in PN skin following treatment with the IL-31 receptor alpha antagonist, nemolizumab. To determine where the biological response of nemolizumab is most prominent, the expression of the two genes encoding the heterodimeric IL-31 receptor, i.e IL-31RA and OSMRB (FIG. 31A, B), was plotted. IL31RA was specifically expressed in fibroblasts and keratinocytes. In contrast, OSMRB was more widely expressed in fibroblasts, keratinocytes, pericytes, and endothelial cells. Strikingly, genes that were differentially upregulated with nemolizumab treatment compared to baseline lesional were predominantly found in the fibroblast cluster, whereas genes that were differentially downregulated localized more broadly to keratinocytes and immune cell subsets, consistent with attenuation of hyperkeratosis and decreased inflammatory responses with treatment (FIG. 31C, D). To study the effects of nemolizumab on the gene expression levels down to the single cell level in PN skin, a gene list was generated from a recently published bulk RNA-seq study, containing genes significantly downregulated by nemolizumab as compared to placebo. Module scores were then calculated using the gene list across all major cellular subtypes in PN skin (FIG. 31D), KC subsets (FIG. 31E, F, G, H),), and FB subsets (FIG. 6F). Consistent with the expression of IL31RA and OSMR, the most pronounced changes were observed in keratinocytes and fibroblasts, particularly inflammatory KCs and the COL11A1+ fibroblasts. These results indicate that nemolizumab treatment reverts the transcription profile from PN towards healthy skin in a broad range of both stromal and immune cell populations, particularly in fibroblasts and keratinocytes, the cell types responsible for the most pronounced histopathological changes in lesional PN skin.

### Discussion

This example provides a detailed insight into the pathogenesis of PN, and the associated tissue-specific and cell-type specific changes that occur in PN skin. Strikingly, changes in PN skin are observed across both immune and stromal cell populations, including keratinocytes, endothelial cells, and most profoundly fibroblasts and fibroblast subpopulations, with increased profibrotic responses being the key distinguishing features of PN from AD, accompanied by an immune shift away from IL-13 and IL-22 responses.

A characteristic histopathologic feature of PN is fibrosis of the papillary dermis with vertically arranged collagen fibers. These findings are consistent with this characteristic, showing a marked increase in dense collagen in the papillary dermis in lesional PN skin by trichrome staining and increased expression of pro-collagen I in the papillary dermis. Moreover, through the single-cell analyses, this example identified the COL11A1+ fibroblast subset as the major source of activated and enriched profibrotic responses, including increased mRNA expression of both collagen I and collagen III. Consistent with their profibrotic function, COL11A1+ fibroblasts were primarily found in the papillary dermis, where the fibrotic response was most marked with either trichrome or pro-collagen I staining indicative of active collagen I biosynthesis. This expansion of the COL11A1+ fibroblast subpopulation is unique to PN and not seen in AD skin. Furthermore, the profibrotic effect of this population was not observed in AD COL11A1+ fibroblasts. Recently a subset of cancer-associated fibroblast (CAF)-like-phenotype was described in AD skin characterized by the expression of WNT5a, tenascin (TCN), and periostin (POSTN) amongst several other genes. In alignment with this publication, periostin (POSTN) and WNT5A were expressed in COL11A1+ fibroblasts in our dataset, along with fibroblast activation protein (FAP), a hallmark marker of CAF. Another recently published paper described CXCL14− IL24+ secretory-papillary dermis fibroblast as being unique to PN skin. Similar IL24+ CXCL14− negative FBs as a small subset with SFRP2+ FBs were observed here, as well as their enrichment in PN skin, but detectable levels in both lesional AD and healthy skin. These FBs expressed increased levels of MMP1 but lower levels of COL1A1 and COL1A2, suggesting that they do not contribute to fibrosis in PN skin.

Of the two components of the heterodimeric IL-31 receptor, IL31RA expression was detected on both keratinocytes and fibroblasts, whereas OSMRB expression was found to be more widespread in different cell populations. This suggests that the two key cell types responding to IL-31 in PN are likely fibroblasts and keratinocytes, which is in keeping with the observation of the transcriptomic shifts driven by nemolizumab that can be attributed to those two cell types.

Notably, this example provided evidence that other cell types including endothelial cells and pericytes also contribute to fibrosis in leshional prurigo nodularis (LPN) skin. Endothelial changes are known in LPN, but the nature of these shifts has not been previously detailed. These data suggest that endothelial cells, likely under the action of proinflammatory and profibrotic cytokines such as TGFB, contribute to extracellular matrix reorganization. TGFb may be an upstream promoter of fibrosis in PN, as TGFb was observed being expressed in a wide range of cell types in PN skin including endothelial cells, fibroblasts, and nerve cells for TGFB1, and fibroblasts and pericytes for TGFB2 and TGFB3. Notably, TGFB2 and TGFB3 have been more strongly implicated in fibrosis than TGFB1.

One of the most characteristic histologic features of PN is the presence of compact orthohyperkeratosis, with irregular epidermal hyperplasia. Keratinocytes demonstrated marked transcriptomic changes in lesional PN skin with the most marked shifts seen in inflammatory keratinocytes, defined by KRT6, KRT16, and KRT17 along with S100A8 and S100A9 expression, and with this analyses indicating a key role of Th2 cytokines (e.g., IL13 and IL22) in this transition, consistent with previous observations. The most enriched biological categories in the inflammatory keratinocyte subset had to do with mitochondrial function and protein translation suggesting the generation of reactive oxygen species and cellular stress that can contribute to inflammatory responses in the skin.

Immune cell infiltrate was prominently observed in lesional PN skin and was characterized by shifts in specific immune cell populations. The most pronounced shifts were observed in macrophage populations, particularly lipid-associated macrophages characterized by the expression of APOE and TREM2. The lipid metabolic products from lipid-associated macrophages have been shown to trigger the production of proinflammatory cytokines in atherosclerosis, which in turn amplify inflammatory responses. These macrophages have recently been implicated in the pathogenesis of acne. T cells were also prominent in PN lesions, with an increased number of cycling T cells, as well as NK, CD8+, and Tregs. The role of these T cell populations in PN skin has not been previously characterized. Furthermore, various stromal cell populations, particularly endothelial and pericytes, had increased expression of various proinflammatory cytokines, chemokines, and adhesion molecules, suggesting an active role in immune trafficking and immune amplification in PN. This includes increased expression of the adhesion molecule ICAM1, E-selectin (SELE), and IL6 in endothelial cells, and CCL2, CCL3, CCL4, CCL13, CCL18, CXCL2, and CXCL12 that were expressed by various cell types (e.g., myeloid cells, pericytes, and endothelial cells) in PN skin. It is noteworthy that CCL2, and IL-6 have established roles in the development of fibrosis. CCL2 is the most potent profibrotic chemokine; through CCR2, CCL2 acts directly on fibroblasts stimulating collagen synthesis. Likewise, IL-6 trans-signaling enhances lung fibroblast proliferation and extracellular matrix protein production.

This data further outlines differences between PN and AD. Notably, the shifts in cell populations in the epidermis are highly similar between both PN and AD with both diseases having a prominent “inflammatory” keratinocyte subset characterized by the expression of pro-inflammatory molecules including S100A8 and S100A9 along with increased expression of the inflammatory keratins KRT6 and KRT16. Changes in the expression of S100A8, S100A9, and KRT16 have been described in AD skin, but their expression has not been addressed in PN skin. Interestingly, the epidermal changes were accompanied by subtle changes in the gene expression in PN vs. AD skin, with immune-related processes such as antimicrobial responses, and regulators of T cell trafficking only observed in LAD, but not LPN keratinocytes. Changes in T cell phenotype were also observed between LPN and LAD skin, particularly within the CD4 effector T cell population, where mRNA expression of IL13 and IL22 was markedly lower in LPN skin compared to LAD skin. IL-22 is known to promote epidermal proliferation and activate innate immune and antimicrobial responses in the skin. IL-13 is a key effector cytokine in AD skin, and the therapeutic target of three biologics: IL-4Ra blocker dupilumab, and the anti-IL13 mAbs lebrikizumab and tralokinumab. These data suggest, that while PN is an inflammatory-driven disease, it may not be centered around IL-13/IL-22 responses to the same degree as AD.

These data also provide information regarding the mechanisms of action of the IL-31 receptor antagonist, nemolizumab. A bulk RNA-seq analysis of LPN skin from patients treated with nemolizumab was performed, and the observed transcriptomic shifts indicating stabilization of extracellular matrix remodeling and normalization of epidermal differentiation. Through cross-referencing the single-cell data with the nemolizumab bulkRNA-seq data, it was possible to show the broad effect of nemolizumab on the abnormal transcriptomic activation of various cell types in PN skin. Thus, the normalization of the pathologic transcriptomic signatures observed in the COL11A1+ fibroblasts and the inflammatory keratinocyte subset likely reflects the observed clinical improvement of PN skin lesions during nemolizumab treatment. These results also validate observations on the molecular and cellular impact of nemolizumab treatment on pathophysiological pillars of PN disease including, inflammation, altered epidermal differentiation and fibrosis.

In summary, these data provide a unique insight into the pathogenesis of PN, highlighting PN as a chronic neuroimmune skin disease with a complex immune-stromal cell crosstalk, promoting and likely driving abnormal keratinocyte proliferation and activation. This is accompanied by a marked shift towards a profibrotic response primarily within the papillary dermis involving activation of COL11A1+ fibroblasts, endothelial cells, and pericytes. Remarkably, these changes are reversible by blocking IL-31 receptor alpha. Therefore, these novel insights expand the understanding of the pathogenesis of PN and the mode of action of anti-IL-31R therapy for this debilitating disease.

Materials and Methods

Human sample acquisition

6 prurigo nodularis, 6 AD patients, and 15 healthy donors were recruited for single-cell RNA sequencing. 6 mm punch biopsies were obtained from affected lesional and non-lesional AD and PN skin. Patients were not on active topical treatment at least 2 weeks prior to enrollment. No patient was on prior systemic treatment. Patients with PN did not have concomitant active AD. The study was approved by the University of Michigan Institutional Review Board (IRB), and all patients gave written consent. The study was conducted according to the Declaration of Helsinki Principles. See patient demographics in the table below:

Patient Demographics

| Sample ID | Age | Sex | Race | Ethnicity |
|---|---|---|---|---|
| PN-001 | 55 | F | W | Non-hispanic |
| PN-002 | 75 | F | W | Non-Hispanic |
| PN-003 | 43 | F | W | Non-Hispanic |
| PN-004 | 69 | M | W | Non-hispanic |
| PN-005 | 28 | F | AA | Non-hispanic |
| PN-006 | 51 | F | AA | Non-Hispanic |
| AD-001 | 32 | F | W | Non-Hispanic |
| AD-002 | 72 | M | W | Non-Hispanic |
| AD-003 | 61 | M | AA | Non-Hispanic |
| AD-004 | 28 | F | W | Non-Hispanic |
| AD-005 | 55 | F | W | Non-Hispanic |
| AD-006 | 29 | F | W | Non-Hispanic |

F = female, M = male, W = white, AA = African American.

Single-Cell RNA-Seq Library Preparation, Sequencing, and Alignment

Generation of single-cell suspensions for scRNA-seq was performed as follows: Skin biopsies were incubated overnight in 0.4% dispase (Life Technologies) in Hank's Balanced Saline Solution (Gibco) at 4° C. The epidermis and dermis were separated. The epidermis was digested in 0.25% Trypsin-EDTA (Gibco) with 10 U/mL DNase I (Thermo Scientific) for 1 hour at 37° C., quenched with FBS (Atlanta Biologicals), and strained through a 70 M mesh. The dermis was minced, digested in 0.2% Collagenase II (Life Technologies) and 0.2% Collagenase V (Sigma) in a plain medium for 1.5 hours at 37° C., and strained through a 70 M mesh. Epidermal and dermal cells were combined in a 1:1 ratio, and the libraries were constructed by the University of Michigan Advanced Genomics Core on the 10× Chromium system with chemistry v3. Libraries were then sequenced on the Illumina NovaSeq 6000 sequencer to generate 150 bp paired-end reads. Data processing including quality control, read alignment (hg38), and gene quantification was conducted using the 10× Cell Ranger software.

Cell Clustering and Cell Type Annotation

The R package Seurat (v4.1.1) was used to cluster the cells in the merged matrix. Cells with less than 500 transcripts or 100 genes or more than 1e5 transcripts or 10% of mitochondrial expression were first filtered out as low-quality cells. The NormalizeData function was used to normalize the expression level for each cell with default parameters. The FindVariableFeatures function was used to select variable genes with default parameters. The ScaleData function was used to scale and center the counts in the dataset. Principal component analysis (PCA) was performed on the variable genes. The RunHarmony function from the Harmony package was applied to remove potential batch effects among samples processed in different batches. Uniform Manifold Approximation and Projection (UMAP) dimensional reduction was performed using the RunUMAP function. The clusters were obtained using the FindNeighbors and FindClusters functions with the resolution set to 0.6. The cluster marker genes were found using the FindAllMarkers function. The cell types were annotated by overlapping the cluster markers with the canonical cell type signature genes. To calculate the disease composition based on cell type, the number of cells for each cell type from each disease condition were counted. The counts were then divided by the total number of cells for each disease condition and scaled to 100 percent for each cell type. Differential expression analysis between any two groups of cells was carried out using the FindMarkers function. All differential expression analyses comparisons were displayed as average log 2 fold-change, and corrected for multiple testing using false-discovery-rate (FDR) adjustment.

Cell Type Sub-Clustering

Sub-clustering was performed on the abundant cell types. The same functions described above were used to obtain the sub-clusters. Sub-clusters that were defined exclusively by mitochondrial gene expression, indicating low quality, were removed from further analysis. The subtypes were annotated by overlapping the marker genes for the sub-clusters with the canonical subtype signature genes. The module scores were calculated using the AddModuleScore function on the intended gene lists. The ECM score was calculated on the genes from the extracellular matrix pathway from the Gene Ontology database. The cytokine score for fibroblast subtypes was calculated on induced genes in fibroblasts after stimulation with TGF-β or IL-4. The Nemolizumab induced or reduced genes were obtained from the previous bulk RNA-seq study by Tsoi et al.. Differentially expressed genes or cluster marker genes were used for enrichment analysis to obtain the potential upstream regulators using Ingenuity Pathway Analysis (QIAGEN Inc., qiagenbioinformatics.com/products/ingenuity-pathway-analysis) or canonical pathways using Enrichr.

Ligand Receptor Interaction Analysis

CellphoneDB (v3) and CellChat were applied for ligand-receptor analysis. Each cell type was separated by its disease classifications (healthy, nonlesional, and lesional), and a separate run was performed for each disease classification. Pairs with p-value >0.05 were filtered out from further analysis. The number of interactions between each cell type pair was then calculated for each condition. To compare the healthy and lesional conditions, the pairs that showed higher interaction score in the lesional condition were used to show the lesional-specific interactions.

Immunohistochemistry Staining

Paraffin embedded tissue sections (lesional and healthy skin) were heated at 60° C. for 30 minutes, de-paraffinized, and rehydrated. Slides were placed in PH9 antigen retrieval buffer and heated at 125° C. for 30 seconds in a pressure cooker water bath. After cooling, slides were treated with 3% H₂O₂ (5 minutes) and blocked using 10% goat serum (30 minutes). Overnight incubation (4° C.) was then performed using anti-human primary antibody. Antibodies used were anti-COL11A1 (ThermoFisher Scientific, cat #PA5-68410), anti-SFRP2 (Lifespan Biosciences, cat #LS-C794043), anti-SFRP4 (Lifespan Biosciences, cat #LC-C408100), anti-TREM2 (ThermoFisher Scientific, cat #PA5-18763), anti-RAMP1 (Abcam, cat #AB64409), anti-CD4 (ThermoFisher Scientific, cat #14-244-82), anti-CD8 (ThermoFisher Scientific, cat #MA5-13473), anti-CD3 (Origene, cat #UM500048). Slides were then washed, and treated with secondary antibody, peroxidase (30 minutes), and diaminobenzidine substrate. Counterstain with Hematoxylin and dehydration was done, and slides were mounted and viewed under the microscope.

Spatial Sequencing Library Preparation

Skin samples were frozen in OCT medium and stored at −80° C. until sectioning. Optimization of tissue permeabilization was performed on 20 m sections using Visium Spatial Tissue Optimization Reagents Kit (10× Genomics, Pleasanton, CA, USA), which established an optimal permeabilization time to be 9 minutes. Samples were mounted onto a Gene Expression slide (10× Genomics), fixed in ice-cold methanol, stained with hematoxylin and eosin, and scanned under a microscope (Keyence, Itasca, IL, USA). Tissue permeabilization was performed to release the poly-A mRNA for capture by the poly(dT) primers that are precoated on the slide and include an Illumina TruSeq Read, spatial barcode, and unique molecular identifier (UMI). Visium Spatial Gene Expression Reagent Kit (10× Genomics) was used for reverse transcription to produce spatially barcoded full-length cDNA and for second-strand synthesis followed by denaturation to allow a transfer of the cDNA from the slide into a tube for amplification and library construction. Visium Spatial Single Cell 3′ Gene Expression libraries consisting of Illumina paired-end sequences flanked with P5/P7 were constructed after enzymatic fragmentation, size selection, end repair, A-tailing, adaptor ligation, and PCR. Dual Index Kit TT Set A (10× Genomics) was used to add unique i7 and i5 sample indexes and generate TruSeq Read 1 for sequencing the spatial barcode and UMI and TruSeq Read 2 for sequencing the cDNA insert, respectively. Libraries were then sequenced on the Illumina NovaSeq 6000 sequencer to generate 150 bp paired-end reads.

Spatial Sequencing Data Analysis

After sequencing, the reads were aligned to the human genome (hg38), and the expression matrix was extracted using the spaceranger pipeline (10× Genomics). Seurat was then used to analyze the expression matrix. Specifically, the SCTransform function was used to scale the data and find variable genes with default parameters. PCA and UMAP were applied for dimensional reduction. The FindTransferAnchors function was used to find a set of anchors between the spatial-seq data and scRNA-seq data, which were then transferred from the scRNA-seq to the spatial-seq data using the TransferData function. These two functions construct a weight matrix that defines the association between each query cell and each anchor. These weights sum to 1 for each spot and were used as the percentage of the cell type in the spot. The ECM score was calculated using the AddModuleScore function on the genes from the extracellular matrix pathway from the Gene Ontology database.

Lengthy table referenced here
US20240141050A1-20240502-T00001
Please refer to the end of the specification for access instructions.

Lengthy table referenced here
US20240141050A1-20240502-T00002
Please refer to the end of the specification for access instructions.

Lengthy table referenced here
US20240141050A1-20240502-T00003
Please refer to the end of the specification for access instructions.

Lengthy table referenced here
US20240141050A1-20240502-T00004
Please refer to the end of the specification for access instructions.

Lengthy table referenced here
US20240141050A1-20240502-T00005
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240141050A1). An electronic copy of the table will also be available from the USPTO upon request and payment of the fee set forth in 37 CFR 1.19(b)(3).

What is claimed:

1. A method of treating or preventing prurigo nodularis (PN) in a subject, comprising administering to a subject with PN an anti-IL-31RA antibody, wherein the subject shows activation of tumor necrosis factor (TNF) signaling in a lesional skin cell compared to a reference level of activation of the TNF signaling or wherein administering the anti-IL-31RA antibody results in a decrease in tumor necrosis factor (TNF) pathway activation.

2. A method of normalizing activation of a tumor necrosis factor (TNF) signaling in a subject with PN, comprising administering to a subject with PN an anti-IL-31RA antibody, wherein the subject shows activation of a tumor necrosis factor (TNF) signaling in a lesional skin cell compared to a reference level of activation of TNF signaling and wherein administration of the anti-IL-31RA antibody normalizes the activation of the TNF signaling.

3. The method of claim 2, wherein normalization is determined about 4 weeks, about 8 weeks, or about 12 weeks after administration of the anti-IL-31RA antibody.

4. The method of claim 1, wherein differential expression was determined by RT-qPCR, RT-PCR, RNA-seq, Northern blotting, Serial Analysis of Gene Expression (SAGE), or DNA or RNA microarrays; or wherein differential expression was determined at protein level by Western blotting, ELISA, surface plasmon resonance, or mass spectrometry.

5. The method of any claim 1, wherein the activation of TNF signaling in the lesional skin cell is higher compared to the reference level of activation.

6. The method of claim 1, wherein the lesional skin cell is a fibroblast

7. The method of claim 1, wherein the reference level is the level of activation is a level of activation of TNF signaling in a skin cell of a person that does not have PN.

8. The method of claim 7, wherein the skin cell of the person that does not have PN is a fibroblast.

9. The method of claim 1 wherein the reference level is the level of activation of TNF signaling in a non-lesional skin cell of the subject.

10. The method of claim 1, wherein the anti-IL-31RA antibody is administered subcutaneously.

11. The method of claim 1, wherein the anti-IL-31RA antibody is administered once per week, once every two weeks, once every three weeks, once every four weeks, once every five weeks, once every six weeks, once every seven weeks, or once every eight weeks.

12. The method of claim 1, wherein the anti-IL-31RA antibody is administered at a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg.

13. The method of claim 1, wherein the anti-IL-31RA antibody is administered according to a flat dosing regimen.

14. The method of claim 1, wherein the anti-IL-31RA antibody is administered according to a loading dose regimen.

15. The method of claim 1, wherein the anti-IL-31RA antibody comprises a heavy chain variable region comprising a HCDR1 comprising SEQ ID NO: 8, a HCDR2 comprising SEQ ID NO: 9, and a HCDR3 comprising SEQ ID NO: 10, and a light chain variable region comprising a LCDR1 comprising SEQ ID NO: 12, a LCDR2 comprising SEQ ID NO: 13, and a LCDR3 comprising SEQ ID NO: 14.

16. The method of claim 1, wherein the anti-IL-31RA antibody is nemolizumab or a fragment or variant thereof.

17. The method of claim 1, wherein the decrease in TNF pathway activation occurs in lesional skin of the subject or in a fibroblast of the subject.

18. The method of claim 1, wherein the treatment further results is:
(a) a decrease in migration of leukocytes or cell movement of leukocytes;
(b) an inhibition of a STAT3 pathway;
(c) an inhibition of a STAT5b pathway;
(d) a downregulation of IL-1 or an IL-1 pathway;
(e) a downregulation of IL-6 or an IL-6 pathway;
(f) a downregulation of VEGF or a VEGF pathway;
(g) a decrease in TGFB1 pathway activation, or
(h) a combination thereof,
wherein (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is determined relative to (i) a control sample obtained from an individual or individuals without PN or (ii) a biological sample obtained from the subject prior to administration of the anti-IL-31RA antibody.

19. The method of claim 18, wherein (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is assessed after about 4 weeks, about 8 weeks, or about 12 weeks after the administration of the anti-IL-31RA antibody.

20. The method of claim 18, wherein (a) the decrease in migration of leukocytes or cell movement of leukocytes; (b) the inhibition of a STAT3 pathway; (c) the inhibition of a STAT5b pathway; (d) the downregulation of IL-1 or an IL-1 pathway; (e) the downregulation of IL-6 or an IL-6 pathway; (f) the downregulation of VEGF or a VEGF pathway; (g) the decrease in TGFB1 pathway activation, or (h) the combination thereof is determined by mass spectrometry performed on one or more biological sample(s) obtained from the subject, and, optionally, wherein the one or more biological sample(s) is a plasma sample or a skin sample.

21. A method of decreasing inflammation in the skin of a subject with prurigo nodularis (PN), comprising administering to a subject with PN an anti-IL-31RA antibody, thereby decreasing inflammation involving tumor necrosis factor (TNF) signaling in the skin, wherein a TNF signaling in skin of the subject is overexpressed relative to a reference level of activation of the TNF signaling, optionally, wherein the TNF signaling is activated in a fibroblast.

22. The method of claim 21, wherein the reference level is (i) the level of activation is a level of activation of TNF signaling in a skin cell of a person that does not have PN, wherein the skin cell of the person that does not have PN is a fibroblast, (ii) the level of activation of TNF signaling in a non-lesional skin cell of the subject.

23. The method of claim 21, wherein the inflammation further involves IL-1 pathway signaling, IL-6 pathway signaling, TGFβ pathway signaling, or any combination thereof.

24. A method of deactivating, decreasing activation, or decreasing a number of COL11A1+ fibroblasts in a subject with prurigo nodularis (PN) or decreasing infiltration of at least one type of immune cell in a skin lesion of a subject with PN, comprising administering to the subject an anti-IL-31RA antibody, wherein administering the anti-IL-31RA antibody results in deactivation, a decrease in activation, or a decrease in number of COL11A1+ fibroblasts in the subject's skin, or wherein administering the anti-IL-31RA antibody results in a decrease in infiltration of at least one type of immune cell in at least one lesion in the subject's skin.

25. A method of decreasing TGFβ expression in at least one cell type in a subject with prurigo nodularis (PN) or decreasing expression at least one inflammatory gene expressed by a keratinocyte in a subject with PN, comprising administering to the subject an anti-IL-31RA antibody, wherein administering the anti-IL-31RA antibody results in a decrease in TGFβ expression in at least one cell type in the subject's skin, or wherein administering the anti-IL-31RA antibody results in a decrease in at least one inflammatory gene expressed by a keratinocyte in the subject's skin.
